(19) United States
(12) Patent Application Publication
(10) Pub. No.: US 2022/0175036 A1
(43) Pub. Date: Jun. 9, 2022

(54) ELECTRONIC CIGARETTES

(71) Applicant: OMEGA LIFE SCIENCE LTD., Migdal Haemeq (IL)

(72) Inventors: Miron HAZANI, Haifa (IL); Jonathan COOKE, Cambridge (GB); Leigh SHELFORD, Cambridge (GB); Andrew NORFOLK, Cambridge (GB); Mike CANE, Royston (GB)

(21) Appl. No.: 17/440,773

(22) PCT Filed: Mar. 24, 2020
(86) PCT No.: PCT/IL2020/050347

§ 371 Date: Sep. 19, 2021

Related U.S. Application Data
(60) Provisional application No. 62/936,633, filed on Nov. 18, 2019.

(30) Foreign Application Priority Data
Mar. 24, 2019 (IL) PCT/IL2019/050325

(51) Int. Cl. A24F 40/44; A24F 40/42; A24F 40/46; A24F 40/60; A24F 40/10; A24F 40/50; A24F 40/48; A61M 15/06; A61M 11/04; A61M 11/00; B05B 17/00; B05B 17/06; H05B 1/02; H05B 3/22

(52) U.S. Cl. CPC A24F 40/44; H05B 2203/003; A24F 40/46; A24F 40/60; A24F 40/10; A24F 40/50; A24F 40/48; A61M 15/06; A61M 11/042; A61M 11/005; B05B 17/0646; B05B 17/0669; H05B 1/0244; H05B 3/22; A61M 2205/0294; H05B 2203/021; A24F 40/42

(57) ABSTRACT

The present disclosure generally relates to the field of aerosol generation devices, and more particularly to electronic cigarettes configured to generation of aerosols from aqueous formulations of nicotine or cannabis products. The present disclosure further provides aqueous cannabinoid compositions for use in the aerosol generation devices.

### FIELD OF THE INVENTION

The present disclosure generally relates to the field of aerosol generation devices, and more particularly to electronic cigarettes configured to generation of aerosols from aqueous formulations of nicotine or cannabis products. The present disclosure further provides aqueous cannabinoid compositions.

### BACKGROUND OF THE INVENTION

Electronic cigarettes typically function as condensation aerosol generators, which operate by vaporizing a liquid such as a nicotine-based composition via heat applied by a heat source. Upon cooling, the vapor condenses to form an aerosol comprising droplets of liquid or particles which can be inhaled by a user through a mouthpiece.

The heated liquid in electronic cigarettes usually includes a composition or mixture of nicotine with humectants, having relatively low latent heat of vaporization, such as propylene glycol (PG) or vegetable glycerin (VG). Said composition is typically referred to as “e-juice”. The liquid mixture is typically drawn into a wicking material that is in contact with a heating element, which may consist a coil of a conducting material to be heated when electric current is driven there through. When not contacted with a liquid, or after the liquid is substantially evaporated the temperature of the coil can reach in some instances a temperature of over 800 degrees Celsius.

In some e-cigarettes, nicotine is provided as a propylene glycol and/or vegetable glycerin formulation, and evaporated together with said solvents. The condensation of nicotine vapor is facilitated by formation of nucleation sites comprising condensed PG and/or VG. Thus, in this type of e-cigarettes PG and/or VG provides the necessary nucleation centers for nicotine condensation

One particular drawback stems from the fact that such products, while carrying a smaller risk than that associated with conventional cigarettes, still present health risks due to the evolution of hazardous compounds arising from heating propylene glycol and vegetable glycerin to elevated temperatures, as well as pyrolysis products of over-heated nicotine.

Condensation of nicotine vapor is facilitated by formation of nucleation sites. Vegetable glycerin used in liquid mixtures of electronic cigarettes provides the necessary nucleation centers for Nicotine condensation.

There is an unmet need for an e-cigarette capable of generating nicotine/THC containing aerosol, which is substantially devoid of hazardous compounds, such as those stemming from the decomposition of PG and VG. Such unmet need also requires that the generation of the aforementioned nicotine-containing aerosol follows condensation of nicotine vapor from condensation centers, of a non-hazardous liquid, such as water.

In addition, there is an unmet need for an e-cigarette capable of delivering an amount of nicotine/THC which suits the particular user requirements or needs.

There is an additional unmet need for an e-cigarette capable of changing the sensory perception of the aerosol inhaled by the user, in accordance with the user's preferences.

### SUMMARY OF THE INVENTION

The present invention generally relates to the field of aerosol generation devices, and more particularly to electronic cigarettes configured to generation of aerosols from aqueous formulations of nicotine or cannabis products. The present disclosure further provides aqueous cannabinoid compositions.

According to some embodiments, there is provided an electronic cigarette comprising a cartridge and an actuator. According to some embodiments, the cartridge comprises a first end and a second end. According to some embodiments, the cartridge comprises an evaporation heater configured to generate heat and to evaporate a liquid from a surface thereof. According to some embodiments, the cartridge further comprises a liquid drawing element. According to some embodiments, the cartridge comprises a liquid container. According to some embodiments, the cartridge comprises an outlet. According to some embodiments, the actuator is having a first end and a second end. According to some embodiments, the actuator comprises a processing unit. According to some embodiments, the first end of the actuator is connectable with the second end of the cartridge. According to some embodiments, the electronic cigarette further comprises a first trigger configured to generate a first trigger activation signal. According to some embodiments, the electronic cigarette further comprises a liquid deposition mechanism comprising the liquid drawing element and the liquid container. According to some embodiments, the electronic cigarette further comprises the liquid drawing element is spaced apart from the evaporation heater in at least a first state of the electronic cigarette. According to some embodiments, the electronic cigarette further comprises the liquid deposition mechanism is configured to transfer a discrete volume of an aqueous formulation from the liquid drawing element to the evaporation heater in a second state of the electronic cigarette. According to some embodiments, the liquid drawing element is in contact with the liquid container in both the first state of the electronic cigarette and the second state of the electronic cigarette.

According to some embodiments, the processing unit is configured to receive at least one operation signal and to control operations of at least one of the evaporation heater and the liquid deposition mechanism upon receiving the at least one operation signal. According to some embodiments, the at least one operation signal comprises the first trigger activation signal.

According to some embodiments, the processing unit is configured to control operations of both the evaporation heater and the liquid deposition mechanism upon receiving the at least one operation signal.

According to some embodiments, the processing unit is configured to control the operation of the liquid deposition mechanism to prevent transfer of liquids from the liquid drawing element to the evaporation heater in the first state of the electronic cigarette.

According to some embodiments, the electronic cigarette is configured to intermittently switch between the first state and the second state thereof, through the processing unit sequentially controlling the operation of the liquid deposition mechanism to (a) prevent transfer of liquids from the liquid drawing element to the evaporation heater in the first state of the electronic cigarette; and (b) transfer a discrete volume of an aqueous formulation from the liquid drawing element to the evaporation heater in the second state of the electronic cigarette.

According to some embodiments, the evaporation heater is flat and comprises a first flat surface facing the outlet and a second flat surface facing the fluid deposition mechanism.

According to some embodiments, the liquid deposition mechanism is configured to transfer a discrete volume of an aqueous formulation from the liquid drawing element to the second flat surface of the evaporation heater in the second state of the electronic cigarette.

According to some embodiments, the evaporation heater is at least partially permeable to the aqueous formulation, and configured to receive the discrete volume of aqueous formulation from the liquid drawing element to the second flat surface thereof, and to evaporate the aqueous formulation through the first flat surface thereof in the second state of the electronic cigarette, such that the evaporated aqueous formulation is released through the outlet.

According to some embodiments, the cartridge comprises a cartridge housing and an evaporation heater support connected thereto.

According to some embodiments, the evaporation heater support is accommodating the evaporation heater, and is made of a low thermal conductivity material.

According to some embodiments, the low thermal conductivity material is selected from the group consisting of ceramics, aluminum silicate, titanium oxide, zirconium oxide, yttrium oxide, molten silicon, silicon dioxide and molten aluminum oxide.

According to some embodiments, the actuator further comprises a power source compartment configured to accommodate a rechargeable battery.

According to some embodiments, the actuator further comprises an actuator power coupling.

According to some embodiments, the cartridge further comprises a cartridge power coupling.

According to some embodiments, upon assembling the cartridge and the actuator to form the electronic cigarette, electric contact is made between the cartridge power coupling and the actuator power coupling.

According to some embodiments, the rechargeable battery is configured to provide electric current to the processing unit and to the actuator power coupling.

According to some embodiments, the evaporation heater comprises an elongated heat conductive coil having a first end, a second end and a main body portion extending there between.

According to some embodiments, the evaporation heater comprises an elongated heat conductive coil having a first end, a second end and a main body portion extending there between in a spiraloid path to form a two-dimensional shape.

According to some embodiments, the two-dimensional shape is having a first flat surface facing the outlet and a second flat surface facing the liquid drawing element.

According to some embodiments, the spiraloid path forms inner tracks between portions of the main body of the elongated heat conductive coil.

According to some embodiments, each of the first end and the second end of the elongated heat conductive coil is connected to an evaporation heater electric contact.

According to some embodiments, each of the evaporation heater electric contacts is in electric contact with a cartridge electric contact.

According to some embodiments, the cartridge electric contact is in electric contact with the cartridge power coupling.

According to some embodiments, the rechargeable battery is configured to provide electric current to each of evaporation heater electric contact through the cartridge electric contact, cartridge power coupling and actuator power coupling.

According to some embodiments, each of the first end and the second end of the elongated heat conductive coil is further connected to a heater resistivity measurement contact.

According to some embodiments, each of the heater resistivity measurement contacts is configured to provide a resistivity measurement signal to the processing unit through output resistivity measurement contacts.

According to some embodiments, the resistivity measurement signal is indicative of the temperature of the evaporation heater.

According to some embodiments, the at least one operation signal comprises the resistivity measurement signal.

According to some embodiments, the actuator further comprises a flow or pressure sensor configured to measure the flow or pressure within the electronic cigarette, and to provide a flow or pressure signal indicative thereof.

According to some embodiments, the at least one operation signal comprises the flow or pressure signal.

According to some embodiments, the discrete volume of the aqueous formulation has a volume in the range of 2 μL to 40 μL.

According to some embodiments, the first trigger is located on the actuator and is selected from the group consisting of a switch, a knob, a dial, a lever, a button, a touch interface, a force sensor, a pressure sensor and a flow sensor.

According to some embodiments, the first trigger comprises a user interface, which provides options to a user for determining at least one control parameter, by which the processing unit controls the liquid deposition mechanism.

According to some embodiments, the at least one control parameter is selected from fluid deposition frequency and fluid deposition duty cycle.

According to some embodiments, the processing unit is configured to control the liquid deposition mechanism in a fluid deposition frequency in the range of 1 Hz to 100 Hz. According to some embodiments, the processing unit is configured to control the liquid deposition mechanism in a fluid deposition frequency in the range of 1 Hz to 30 Hz.

According to some embodiments, the at least one control parameter comprises at least two separate fluid deposition frequencies, wherein each frequency is in the range of 1 Hz to 100 Hz. According to some embodiments, the at least one control parameter comprises at least two separate fluid deposition frequencies, wherein each frequency is in the range of 1 Hz to 30 Hz.

According to some embodiments, the processing unit is configured to control the liquid deposition mechanism in a duty cycle in the range of 10% to 50%.

According to some embodiments, the at least one control parameter comprises at least two separate duty cycles, each in the range of 10% to 50%.

According to some embodiments, the user interface is located on the actuator.

According to some embodiments, the user interface is located on a remote device in communication with the processing unit through internet connectivity.

According to some embodiments, the liquid deposition mechanism further comprises a biasing element, configured to trigger a dislocation of at least a portion of the liquid drawing element between a first position in the first state of the electronic cigarette and a second position in the second state of the electronic cigarette.

According to some embodiments, the liquid drawing element is spaced apart from the evaporation heater in the first position.

According to some embodiments, the liquid drawing element is in contact with the evaporation heater in the second position.

According to some embodiments, the biasing element is positioned between the liquid drawing element and the second end of the actuator.

According to some embodiments, the biasing element is configured to dislocate the portion of the liquid drawing element from the first position in the first state of the electronic cigarette in the direction of the first end of the cartridge, towards the second position in the second state of the electronic cigarette.

According to some embodiments, the biasing element is further configured to trigger dislocation of the liquid drawing element from the second position in the second state of the electronic cigarette in the direction of the second end of the actuator, towards the first position in the first state of the electronic cigarette.

According to some embodiments, the liquid drawing element is flexible and comprises at least first portion and a second portion.

According to some embodiments, the second portion of the liquid drawing element is in contact with the liquid container in both the first state of the electronic cigarette and the second state of the electronic cigarette.

According to some embodiments, the second portion of the liquid drawing element is in contact with an internal compartment of the liquid container in both the first state of the electronic cigarette and the second state of the electronic cigarette.

According to some embodiments, the biasing element is configured to trigger a dislocation of the first portion of the liquid drawing element between the first position in the first state of the electronic cigarette and the second position in the second state of the electronic cigarette.

According to some embodiments, the first portion of the liquid drawing element is spaced apart from the evaporation heater in the first position.

According to some embodiments, the first portion of liquid drawing element is in contact with the evaporation heater in the second position.

According to some embodiments, the liquid drawing element comprises fabric, cloth, wool, felt, sponge, foam, cellulose, yarn, microfiber or a combination thereof.

According to some embodiments, the liquid drawing element comprises a wick.

According to some embodiments, the biasing element comprises a solenoid actuator, a rod and a solenoid plunger head.

According to some embodiments, rod has a first end and a second end, wherein the second end is connected to the solenoid actuator, and the first end is connected to the solenoid plunger head.

According to some embodiments, the solenoid actuator is configured to dislocate the solenoid plunger head between a first position and a second position.

According to some embodiments, in the second state of the electronic cigarette, the solenoid plunger head is in the second position thereof and is pressing the portion of the liquid drawing element against the evaporation heater, and in the first state of the electronic cigarette, the solenoid plunger head is in the first position thereof and the liquid drawing element is spaced apart from the evaporation heater.

According to some embodiments, the actuator further comprises a liquid deposition mechanism housing.

According to some embodiments, the liquid deposition mechanism housing accommodates the solenoid actuator.

According to some embodiments, the rod extends from the solenoid actuator in the direction of the first end of the cartridge.

According to some embodiments, when the cartridge and the actuator are assembled, the solenoid plunger head resides inside the cartridge, between the solenoid actuator and the evaporation heater.

According to some embodiments, the solenoid actuator is configured to receive electric current and to generate axial movement of the solenoid plunger head upon receiving the electric current.

According to some embodiments, the axial movement is along an axis perpendicular to the evaporation heater, between the first position of the solenoid plunger head in the first state of the electronic cigarette and the second position of the solenoid plunger head in the second state of the electronic cigarette.

According to some embodiments, the processing unit is configured to drive the current to the solenoid actuator and to control the rate of the axial movement of the solenoid plunger head by controlling the current.

According to some embodiments, the liquid deposition mechanism further comprises a spraying mechanism.

According to some embodiments, the spraying mechanism is located within the cartridge and configured to create a spray from the aqueous formulation.

According to some embodiments, the spraying mechanism is in contact with the liquid drawing element and spaced apart from the evaporation heater in both the first state of the electronic cigarette and the second state of the electronic cigarette.

According to some embodiments, the spraying mechanism is located between the liquid drawing element and the evaporation heater.

According to some embodiments, in the first state of the electronic cigarette the spraying mechanism does not create a spray.

According to some embodiments, in the second state of the electronic cigarette, the spray is sprayed from the spraying mechanism in the direction of the first end of the actuator and contacts the evaporation heater.

According to some embodiments, the liquid deposition mechanism further comprises a liquid deposition mechanism housing.

According to some embodiments, the spraying mechanism comprises a piezo disc.

According to some embodiments, the piezo disc is configured to create the spray from the aqueous formulation.

According to some embodiments, the piezo disc is in contact with the liquid drawing element and spaced apart from the evaporation heater in both the first state of the electronic cigarette and the second state of the electronic cigarette.

According to some embodiments, the piezo disc is accommodated within the liquid deposition mechanism housing.

According to some embodiments, the liquid deposition mechanism housing comprises a piezo slot, and the piezo disc is accommodated within the piezo slot.

According to some embodiments, the piezo disc is configured to convert electric current to vibrations having resonant frequency, which creates the spray from the aqueous formulation.

According to some embodiments, the resonant frequency is in the range of 100 KHz to 10 MHz-KHz.

According to some embodiments, the processing unit is configured to drive the current to the piezo disc and to control the spraying by controlling the current.

According to some embodiments, the piezo disc is made of metal.

According to some embodiments, the piezo disc comprises a first flat surface facing the evaporation heater and a second flat surface in contact with the liquid drawing element.

According to some embodiments, the piezo disc is perforated disc.

According to some embodiments, upon application of electric current through the piezo disc in the second state of the electronic cigarette, the piezo disc is configured to convert the aqueous formulation in contact with the second flat surface thereof to the spray, which is released through the perforations of the piezo disc from the first surface thereof.

According to some embodiments, the spray is released from the first surface of the piezo disc in the direction the direction of the first end of the actuator and contacts the evaporation heater.

According to some embodiments, there is provided an electronic cigarette comprising a cartridge and an actuator. According to some embodiments, the cartridge is having a first end and a second end. According to some embodiments, the cartridge comprises an evaporation heater configured to generate heat and to evaporate a liquid from a surface thereof.

According to some embodiments, the cartridge comprises a liquid container having an internal compartment. According to some embodiments, the cartridge comprises a liquid drawing element having a mobile first portion and a stationary second portion, wherein the stationary second portion is in contact with the internal compartment of the liquid container.

According to some embodiments, the cartridge comprises an outlet.

According to some embodiments, the actuator is having a first end and a second end.

According to some embodiments, the actuator comprises a processing unit.

According to some embodiments, the first end of the actuator is connectable with the second end of the cartridge.

According to some embodiments, the electronic cigarette further comprises a first trigger configured to generate a first trigger activation signal.

According to some embodiments, the electronic cigarette comprises a liquid deposition mechanism comprising the liquid drawing element, the liquid container and a biasing element.

According to some embodiments, biasing element is configured to trigger a dislocation of the first portion of the liquid drawing element between a first position and a second position.

According to some embodiments, the liquid drawing element is spaced apart from the evaporation heater in the first position.

According to some embodiments, the first portion of the liquid drawing element is in contact with the evaporation heater in the second position.

According to some embodiments, the processing unit is configured to receive at least one operation signal and to control operations of at least one of the evaporation heater and the liquid deposition mechanism upon receiving the at least one operation signal.

According to some embodiments, the at least one operation signal comprises the first trigger activation signal.

According to some embodiments, the biasing element comprises a solenoid actuator, a rod and a solenoid plunger head.

According to some embodiments, the rod has a first end and a second end, wherein the second end is connected to the solenoid actuator, and the first end is connected to the solenoid plunger head.

According to some embodiments, the solenoid actuator is configured to dislocate the solenoid plunger head between a first position and a second position.

According to some embodiments, in a second state of the electronic cigarette, the solenoid plunger head is in the second position thereof and is pressing the mobile first portion of the liquid drawing element against the evaporation heater, and in a first state of the electronic cigarette, the solenoid plunger head is in the first position thereof and the liquid drawing element is spaced apart from the evaporation heater.

According to some embodiments, there is provided an electronic cigarette comprising a cartridge and an actuator. According to some embodiments, the cartridge is having a first end and a second end. According to some embodiments, the cartridge comprises an evaporation heater configured to generate heat and to evaporate a liquid from a surface thereof.

According to some embodiments, the cartridge comprises a liquid drawing element.

According to some embodiments, the cartridge comprises a liquid container comprising an internal compartment.

According to some embodiments, the cartridge comprises a spraying mechanism.

According to some embodiments, the cartridge comprises an outlet.

According to some embodiments, the actuator comprises a processing unit.

According to some embodiments, the actuator is having a first end and a second end.

According to some embodiments, the first end of the actuator is connectable with the second end of the cartridge.

According to some embodiments, the electronic cigarette further comprises a first trigger configured to generate a first trigger activation signal.

According to some embodiments, the liquid drawing element is in contact with the spraying mechanism and the internal compartment of the liquid container.

According to some embodiments, the spraying mechanism is positioned between the evaporation heater and the liquid drawing element.

According to some embodiments, each one of the liquid drawing element, the liquid container and the spraying mechanism is spaced apart from the evaporation heater.

According to some embodiments, the spraying mechanism is configured to deliver a spray of an aqueous formulation to the evaporation heater.

According to some embodiments, the processing unit is configured to receive at least one operation signal and to control operations of at least one of the evaporation heater and the liquid deposition mechanism upon receiving the at least one operation signal.

According to some embodiments, the at least one operation signal comprises the first trigger activation signal.

According to some embodiments, the liquid deposition mechanism further comprises a liquid deposition mechanism housing.

According to some embodiments, the spraying mechanism comprises a piezo disc.

According to some embodiments, the piezo disc is configured to create the spray from an aqueous formulation.

According to some embodiments, the piezo disc is in contact with the liquid drawing element and spaced apart from the evaporation heater.

According to some embodiments, the liquid deposition mechanism housing comprises a piezo slot, and the piezo disc is accommodated within the piezo slot.

According to some embodiments, the piezo disc is made of metal.

According to some embodiments, the piezo disc comprises a first flat surface facing the evaporation heater and a second flat surface in contact with the liquid drawing element.

According to some embodiments, piezo disc is perforated disc.

According to some embodiments, upon application of electric current through the piezo disc in a second state of the electronic cigarette, the piezo disc is configured to convert the aqueous formulation in contact with the second flat surface thereof to the spray, which is released through the perforations of the piezo disc from the first surface thereof towards the evaporation heater.

According to some embodiments, in a first state of the electronic cigarette the spraying mechanism does not create a spray.

According to some embodiments, there is provided cannabinoid composition for use in the administration of a cannabinoid via inhalation, the cannabinoid composition comprises an aqueous solution comprising at least one cannabinoic acid or a salt thereof, wherein the aqueous solution has a pH of at least 9.

According to some embodiments, the administration of the cannabinoid via inhalation comprises generating an inhalable aerosol upon heating the cannabinoid composition in any one of the electronic cigarettes disclosed herein.

According to some embodiments, the inhalable aerosol has a pH in the range of 5.5 to 7.5.

According to some embodiments, the at least one cannabinoic acid or salt thereof comprises THCA or a salt thereof at a concentration in the range of 4% to 6% w/w.

According to some embodiments, the is provided a method of delivering a cannabinoid to a user of an electronic cigarette via inhalation, the method comprising the steps of: (i) providing a cannabinoid composition comprising an aqueous solution comprising at least one cannabinoic acid or salt thereof, wherein the aqueous solution has a pH of at least 9; and (ii) aerosolizing the cannabinoid composition of step (a) with an electronic cigarette, to form an inhalable aerosol, wherein the inhalable aerosol is inhaled by the user of the electronic cigarette. According to some embodiments, the electronic cigarette is the electronic cigarette disclosed herein.

According to some embodiments, there is provide an aerosol composition comprising tetrahydrocannabinol (THC) at a total weight of 1-8% w/w based on the total weight of the aerosol composition, and water 70-99% w/w based on the total weight of the aerosol composition, wherein the aerosol comprising droplets having an mass median aerodynamic diameter (MMAD) of at most 50 microns, wherein the aerosol composition further comprises tetrahydrocannabinolic acid (THCA), wherein the aerosol composition is formed by aerosolizing a cannabinoid composition using the electronic cigarette disclosed herein, wherein the cannabinoid composition comprises an aqueous solution comprising THCA, wherein the aqueous solution has a pH of at least 9.

Other objects, features and advantages of the present invention will become clear from the following description, examples and drawings.

### BRIEF DESCRIPTION OF THE DRAWINGS

FIG. 1 constitutes a schematic view of an electronic cigarette comprising a cartridge and an actuator, when connected, according to some embodiments.

FIG. 2 constitutes a schematic view of an electronic cigarette comprising a cartridge and an actuator, when separated, according to some embodiments.

FIGS. 3A and 3B constitute schematic views of an electronic cigarette comprising a cartridge and an actuator, when connected, in a first state of the electronic cigarette (FIG. 3A) and, in a second state of the electronic cigarette (FIG. 3B).

FIGS. 3C and 3D constitute schematic views of an electronic cigarette comprising a cartridge and an actuator, when connected, in a first state of the electronic cigarette (FIG. 3A) and, in a second state of the electronic cigarette (FIG. 3B).

FIG. 4 constitutes a schematic view of an electronic cigarette comprising a cartridge and an actuator, when connected, according to some embodiments.

FIG. 5 constitutes a schematic view of an electronic cigarette comprising a cartridge and an actuator, when separated, according to some embodiments.

FIGS. 6A-C constitute different views of an actuator of an electronic cigarette, according to some embodiments.

FIG. 7 constitutes a view in prospective of an evaporation heater of an electronic cigarette, according to some embodiments.

FIG. 8 constitutes a view in prospective of an evaporation heater of an electronic cigarette, according to some embodiments.

FIGS. 9A and 9B constitute views in prospective of two evaporation heaters of an electronic cigarette, wherein each evaporation heater comprises a heat conductive coil, and the evaporation heater of FIG. 9B has a longer heat conductive coil than the evaporation heater of FIG. 9A.

FIG. 10 constitutes a view in prospective of an evaporation heater of an electronic cigarette, according to some embodiments.

FIGS. 11A-E constitute different views of an evaporation heater of an electronic cigarette, housed within a support, according to some embodiments

FIGS. 12A and 12B constitute views in prospective of a processing unit assembly of an electronic cigarette with (FIG. 12A) and without (FIG. 12B) a piezo inductor, according to some embodiments.

FIG. 13 constitutes a view in prospective of a piezo disc 180 of an electronic cigarette, according to some embodiments.

FIGS. 14A and 14B constitute zoomed-in views in prospective of a cartridge of an electronic cigarette, according to some embodiments.

FIG. 15 constitute a zoomed-in view in prospective of a cartridge of an electronic cigarette, according to some embodiments.

FIGS. 16A-C constitute different views of a cartridge of an electronic cigarette, according to some embodiments.

FIG. 17 constitutes a top cross sectional view of a cartridge of an electronic cigarette, according to some embodiments.

FIGS. 18A-B constitute cross sectional views of a portion of cartridge of an electronic cigarette, according to some embodiments.

FIGS. 19A-B constitute cross sectional views of a portion of cartridge of an electronic cigarette, according to some embodiments.

FIG. 20 constitutes view in prospective of a portion of cartridge of an electronic cigarette, according to some embodiments.

FIGS. 21A-B constitute different views of an actuator of an electronic cigarette, according to some embodiments.

FIG. 22 constitutes a view in prospective of a battery of an electronic cigarette, according to some embodiments.

FIG. 23 constitutes a top cross sectional view of an actuator of an electronic cigarette, according to some embodiments.

FIG. 24 constitutes a view in prospective different elements of an electronic cigarette, according to some embodiments.

FIG. 25 constitutes a zoomed-in view in prospective of a cartridge of an electronic cigarette, according to some embodiments.

FIG. 26 constitutes a top cross sectional view of a cartridge of an electronic cigarette, according to some embodiments.

FIGS. 27A-B constitute cross sectional views of a portion of cartridge of an electronic cigarette, according to some embodiments.

FIGS. 28A-C constitute schematic illustrations of an electronic cigarette, according to some embodiments.

FIGS. 29A-C constitute schematic illustrations of a top view of parts of an electronic cigarette, according to some embodiments.

FIGS. 30A-B constitute schematic illustrations of an electronic cigarette, according to some embodiments.

FIGS. 31A-B constitute schematic illustrations of an electronic cigarette, according to some embodiments.

FIGS. 32A-B constitute schematic illustrations of an electronic cigarette, according to some embodiments.

FIG. 33 shows two overlaying HPLC chromatograms; a chromatogram of the aqueous formulation disclosed herein (dotted line); and a chromatogram of an elution of an aerosol produced from aerosolizing the aqueous formulation disclosed herein (full line).

FIG. 34 is a chart representing Mass Distribution on Impactor parts in an aerosol depicting the relative mass of an aerosol produced from aerosolizing the aqueous formulation disclosed herein, in each particle diameter size group.

FIG. 35 shows Mass Distribution on Impactor parts in an aerosol produced from aerosolizing the aqueous formulation disclosed herein.

### DETAILED DESCRIPTION

Provided herein are electronic cigarettes In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout. Throughout the figures of the drawings, different superscripts for the same reference numerals are used to denote different embodiments of the same elements. Embodiments of the disclosed devices and systems may include any combination of different embodiments of the same elements. Specifically, any reference to an element without a superscript may refer to any alternative embodiment of the same element denoted with a superscript. Components having the same reference number followed by different lowercase letters may be collectively referred to by the reference number alone. If a particular set of components is being discussed, a reference number without a following lowercase letter may be used to refer to the corresponding component in the set being discussed.

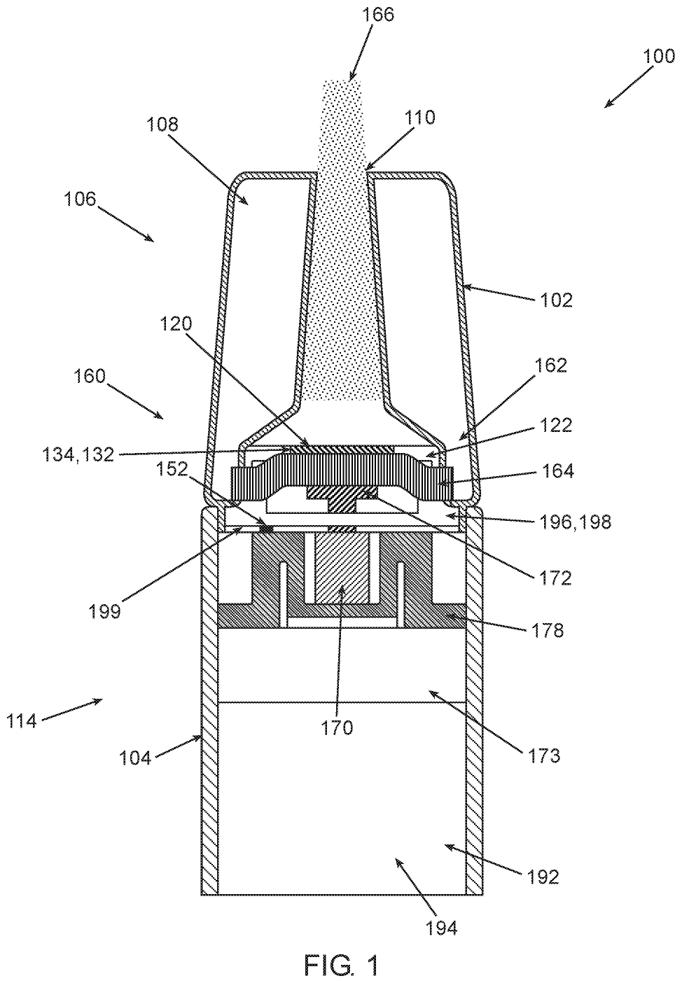

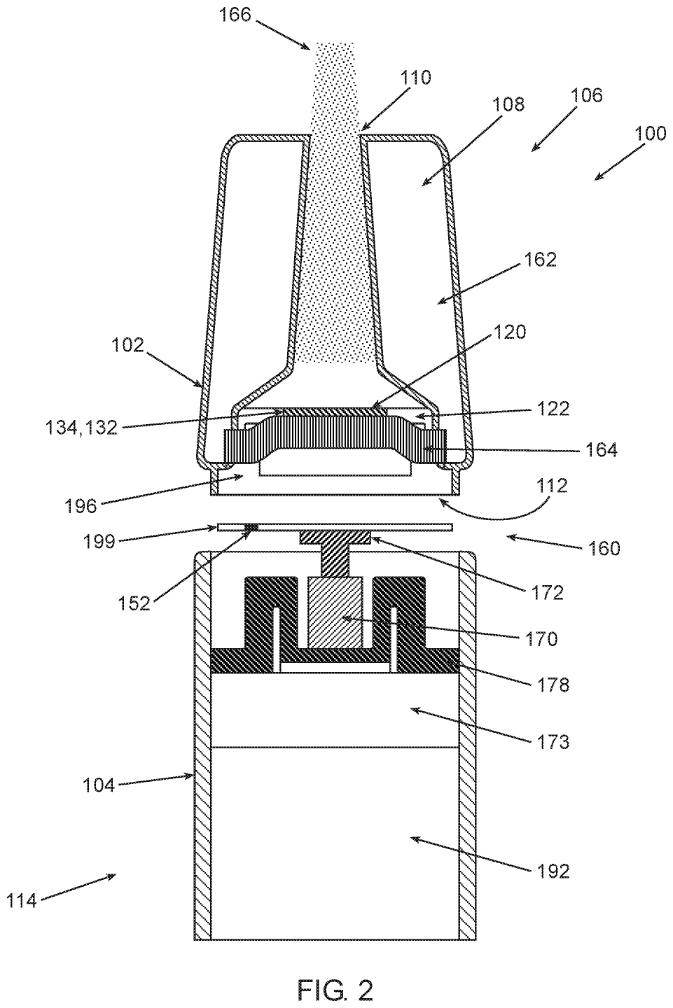

Reference is now made to FIG. 1 and FIG. 2. FIG. 1 and FIG. 2 constitute schematic illustration of an electronic cigarette 100, according to some embodiments. The terms “electronic cigarette” and “e-cigarette” as used herein, are interchangeable and refer to a device configured to produce a vapor or aerosol from a liquid or solid composition and comprises at least a heating unit for heating the composition, and an outlet for delivering out the formed aerosol composition for a user to inhale, typically through a mouthpiece.

Electronic cigarette 100 comprises a cartridge 106 comprising a cartridge housing 102 and a cartridge internal compartment 108. Electronic cigarette 100 further comprises an actuator 114 comprising an actuator housing 104. Electronic cigarette 100 further comprises an outlet 110, an evaporation heater 120, a first trigger 140, a liquid deposition mechanism 160 and a processing unit 190.

According to some embodiments, outlet 110 is formed on cartridge housing 102. According to some embodiments, electronic cigarette 100 is configured to produce an aerosol 166, and outlet 110 is configured to deliver aerosol 166 out of electronic cigarette 100. It is to be understood that the objective of electronic cigarettes is generally to produce an aerosol, and to deliver it through the outlet or mouthpiece of the electronic cigarette, through a mouth of an electronic cigarette user to the respiratory system of the user.

According to some embodiments, outlet 110 is connected to a mouthpiece. According to some embodiments, outlet 110 is mechanically connected to a mouthpiece. According to some embodiments, the mouthpiece is detachable.

According to some embodiments, evaporation heater 120 is accommodated within cartridge internal compartment 108.

Generally, electronic cigarettes, including electronic cigarette 100 have an elongated shape, e.g. as depicted in FIGS. 1-6, 28, 30. Within the context of this specification, the term “longitudinal” refer to the direction of elongation of electronic cigarette 100. The term “longitudinal axis” refers to the linear axis along the longitudinal direction.

Within the context of this specification the terms “top”, “above”, “up” and “upwards” generally refer, longitudinally, to the side or end of any device or a component of a device, which is closer to outlet 110.

Within the context of this specification the terms “top”, “above”, “up”, “upwards” are interchangeable with the term “proximal” referring to proximity to the mouthpiece or the user using e-cigarette.

Within the context of this specification the terms “bottom”, “below”, “down”, “under” and “downwards” generally refer, longitudinally, to the side or end of any device or a component of a device, which is farther than outlet 110.

Within the context of this specification the terms “bottom”, “below”, “down”, “under” and “downwards” are interchangeable with the term “distal”.

Generally, during operation of electronic cigarette 100, liquid deposition mechanism 160 delivers a discrete, known volume of liquid, or a plurality of discrete, known volumes of liquid, intermittently to evaporation heater 120. Evaporation heater 120 is heated to an elevated temperature, which rapidly evaporates the discrete volume of liquid and generates aerosol 166 therefrom, according to some embodiments.

The intermittent nature of liquid delivery from liquid deposition mechanism 160 to evaporation heater 120 has benefits, especially when aerosolizing aqueous formulations, and is achieved using a two-state liquid deposition mechanism 160, according to some embodiments. By referring the two-state liquid deposition mechanism, it is not meant that liquid deposition mechanism 160 cannot have more than two states, such as intermediate states. Furthermore, each of the states described below may have non-identical forms and/or mechanisms of action.

Specifically, according to some embodiments, in a first state of electronic cigarette 100, liquid deposition mechanism 160 is spaced apart from evaporation heater 120, such that liquid is not deposited onto evaporation heater 120, when electronic cigarette 100 is in the first state of operation.

In a second state of electronic cigarette 100, according to some embodiments, liquid deposition mechanism 160 is delivering a discrete volume of liquid onto evaporation heater 120, and the discrete volume of liquid is evaporated and subsequently aerosolized, due to evaporation heater 120 being in an elevated evaporation temperature. In the second state of electronic cigarette 100, liquid deposition mechanism 160 may be spaced apart from evaporation heater 120 and deposit liquid thereon from distance, according to some embodiments. Alternatively, according to some embodiments, in the second state of electronic cigarette 100, an element of liquid deposition mechanism 160 may approach evaporation heater 120, such that contact is established between evaporation heater 120 and the element of liquid deposition mechanism 160. In this alternative, the contact between evaporation heater 120 and an element of liquid deposition mechanism 160 enables the delivery of a discrete volume of liquid onto evaporation heater 120, and the discrete volume of liquid is evaporated by the heat of evaporation heater 120 and subsequently aerosolized to form aerosol 166.

Without wishing to be bound by any theory or mechanism of action, one of the challenges of evaporating aqueous compositing in electronic cigarettes stems from the pronounce Leidenfrost effect on water, as detailed herein. In order the circumvent the obstacles associated with the Leidenfrost effect on water, preferably discrete portions of relatively thin layers of liquid need to be dispersed over evaporation heater 120, as discrete thin layers generally evaporate quickly. Furthermore, is was found for the first time that evaporation of discrete thin film of liquid results in formation of small aerosolized droplet, compared to large droplets formed when soaking heaters in liquids. Thus, in contrast with conventional e-cigarettes, which evaporate PG or VG-containing formulations that allow the soaking of their evaporation elements in the formulation, electronic cigarette 100 comprises evaporation heater 120, which ‘dries’ quickly. Evaporation heater 120 evaporates the thin layer of liquid and dries quickly, since the thin layer contains small amount of material.

As used herein the terms “aerosol”, “aerosolized composition” or “aerosolized drug” refer to a dispersion of solid or liquid particles in a gas. As used herein “aerosol”, “aerosolized composition” or “aerosolized drug” may be used generally to refer to a material that has been vaporized, nebulized, being in a form of spray or jet or otherwise converted from a solid or liquid form to an inhalable form including suspended solid or liquid drug particles. According to some embodiments, the drug particles include nicotine particles. According to some embodiments, the drug particles include cannabinoid particles.

As used herein, the terms “vaporization” and “evaporation” are interchangeable.

It is to be understood that in contrast with known e-cigarettes, which employ liquid deposition mechanisms, in which the evaporation surface is in continuous contact with a large reservoir of nicotine/cannabinoid formulation (i.e. the evaporation medium is typically ‘soaked’ with a VG or PG nicotine/cannabinoid formulation), the liquid deposition mechanism disclosed herein delivers small and discrete amounts of aqueous nicotine/cannabinoid formulations. Without wishing to be bound by any theory or mechanism of action, when aqueous solutions of nicotine/cannabinoid(s) are loaded into the ‘soaking’ liquid deposition mechanisms of the known devices, the heat transfer from the heating element to the formulation maintains the formulation temperature at around the boiling point of water. Since the boiling temperature of water (100° C.) is not sufficient for effective evaporation of nicotine or THC, the liquid deposition mechanisms known to date fail to efficiently evaporate aqueous nicotine/cannabinoid formulations. In contrast, it was found that delivery of discrete and small amounts of aqueous nicotine/cannabinoid formulations to the evaporation heater, as disclosed herein, provides sufficient heating of the formulations, thereby enabling substantial evaporation of nicotine. Specifically, the boiling of discrete volumes entails breaking the continuous delivery (performed by standard electronic cigarettes) into a series of discrete aerosolization events. Each event involves deposition of the aqueous formulation, water evaporation and nicotine/cannabinoid evaporation. Thus, the discrete boiling approach disclosed herein combines rapid water evaporation and rapid temperature rise thereafter to an evaporation temperature of nicotine/THC, thereby achieving quick and effective evaporation of nicotine or THC.

The terms “effective evaporation” and “substantial evaporation” are interchangeable and are intended to mean that at least 50%, at least 60%, at least 70%, at least 75%, at least 80%, at least 85%, at least 90%, at least 95%, least 97%, least 98%, least 99%, least 99.5% or least 99.9% of the liquid is transformed from liquid to gaseous state.

According to some embodiments, evaporation heater 120 is located longitudinally between outlet 110 and liquid deposition mechanism 160. Specifically, as defined above with respect to directions, evaporation heater 120 is located above liquid deposition mechanism 160, and outlet 110 is located above evaporation heater 120. Therefore, upon operation of electronic cigarette 100 from the first state to the seconds state, liquid deposition mechanism 160 deposits the discrete volume of liquid on the bottom of evaporation heater 120, and vapor is released from the top of evaporation heater 120.

According to some embodiments, evaporation heater 120 is flat and comprises a first surface facing outlet 110 and a second surface facing liquid deposition mechanism 160.

According to some embodiments, electronic cigarette 100 comprises compartment of processing unit assembly 173, accommodated within actuator 114. According to some embodiments, compartment of processing unit assembly 173 comprises processing unit assembly 174. According to some embodiments, processing unit assembly 174 comprises processing unit 190. According to some embodiments, electronic cigarette 100 comprises processing unit 190, accommodated within actuator 114.

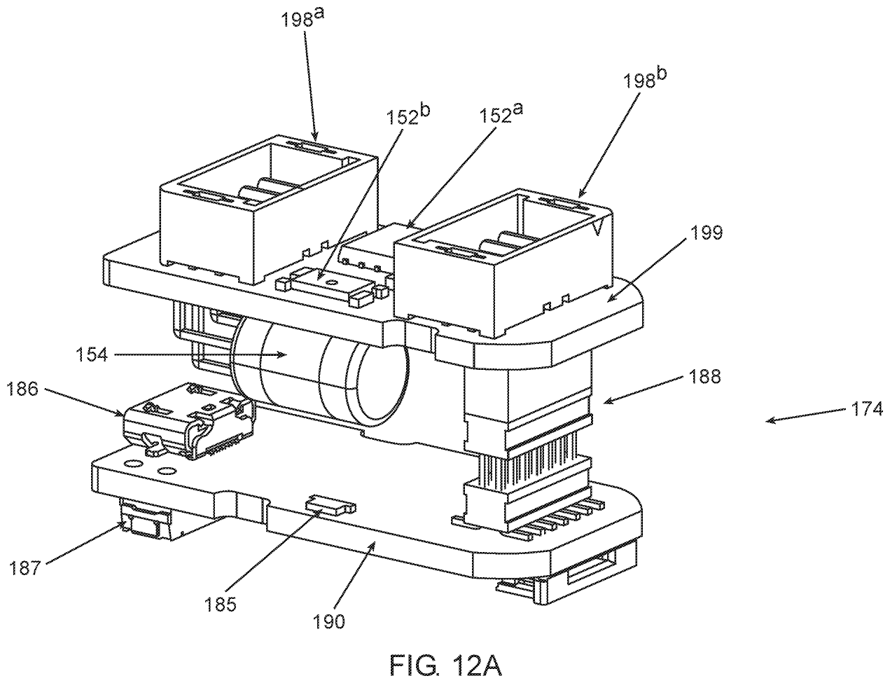

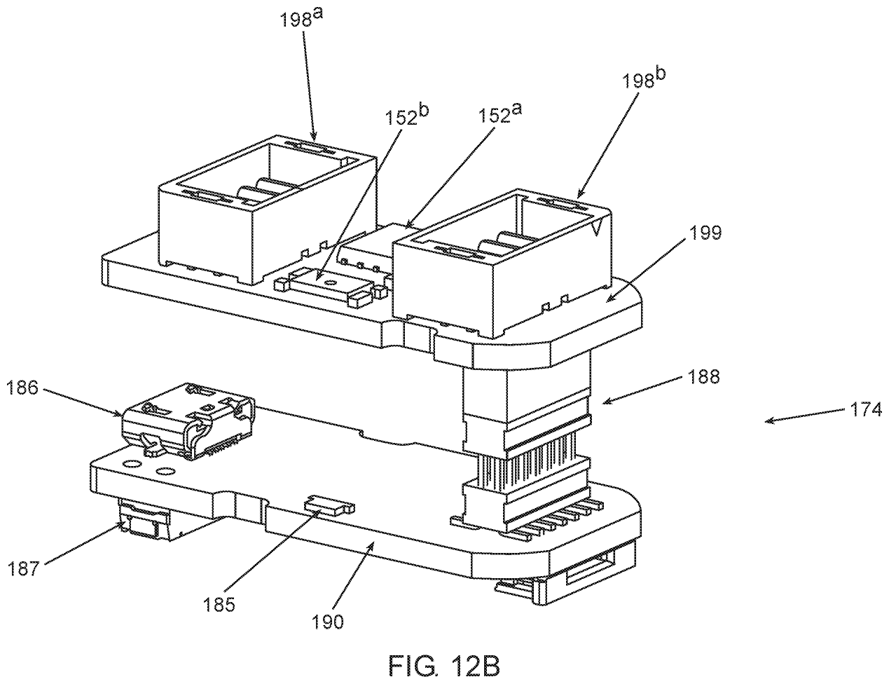

Compartment of processing unit assembly 173 is shown in FIGS. 1-2. The contents of compartment of processing unit assembly 173, including processing unit 190 are elaborated when referring to FIGS. 12A and 12B.

According to some embodiments, processing unit 190 is configured to receive signals from first trigger 140. According to some embodiments, first trigger 140 is configured to generate at least a first trigger activation signal. According to some embodiments, evaporation heater 120 is configured to generate heat when first trigger 140 generates the first trigger activation signal.

According to some embodiments, liquid deposition mechanism 160 is configured to control the operation of evaporation heater 120. According to some embodiments, processing unit 190 is configured to activate evaporation heater 120 upon receiving first trigger activation signal from first trigger 140. According to some embodiments, processing unit 190 is configured to deactivate at least one heating element.

According to some embodiments, processing unit 190 is configured to control operation of liquid deposition mechanism 160. According to some embodiments, processing unit 190 is configured to control operation of liquid deposition mechanism 160, such that liquid deposition mechanism 160 delivers a discrete volume of liquid to evaporation heater 120. According to some embodiments, processing unit 190 is configured to operate liquid deposition mechanism 160 to perform a transition from the first state to the second state of electronic cigarette 100. According to some embodiments, processing unit 190 is configured to operate liquid deposition mechanism 160 to perform a transition from the second state to the first state of electronic cigarette 100. According to some embodiments, processing unit 190 is configured to operate liquid deposition mechanism 160 to perform a transition from the first state to the second state and to perform a transition from the second state to the first state of electronic cigarette 100 consecutively, so as to provide a discrete volume of liquid from liquid deposition mechanism 160 to evaporation heater 120. According to some embodiments, processing unit 190 is configured to operate liquid deposition mechanism 160 to perform the following sequence of operations consecutively:
(a) a transition of liquid deposition mechanism 160 from the first state to the second state of electronic cigarette 100;
(b) maintenance of liquid deposition mechanism 160 in the second state for a predetermined period of deposition time; wherein during the predetermined period of deposition time, liquid deposition mechanism 160 is configured to deliver a discrete volume of liquid to evaporation heater 120; and
(c) a transition of liquid deposition mechanism 160 from the second state to the first state of electronic cigarette 100.

According to some embodiments, operation (b) is the only operation in which liquid deposition mechanism 160 is configured to deliver a liquid to evaporation heater 120.

According to some embodiments, processing unit 190 is configured to perform the sequence of operations a plurality of times upon receiving the first activation signal.

According to some embodiments, processing unit 190 is configured to activate liquid deposition mechanism 160 upon receiving first trigger activation signal from first trigger 140. According to some embodiments, processing unit 190 is configured to deactivate liquid deposition mechanism 160.

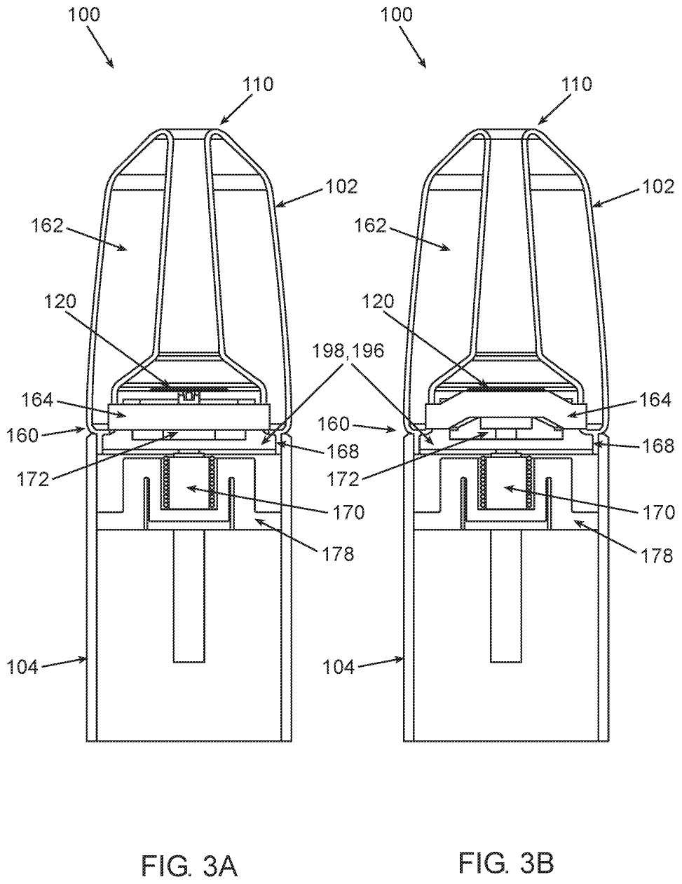

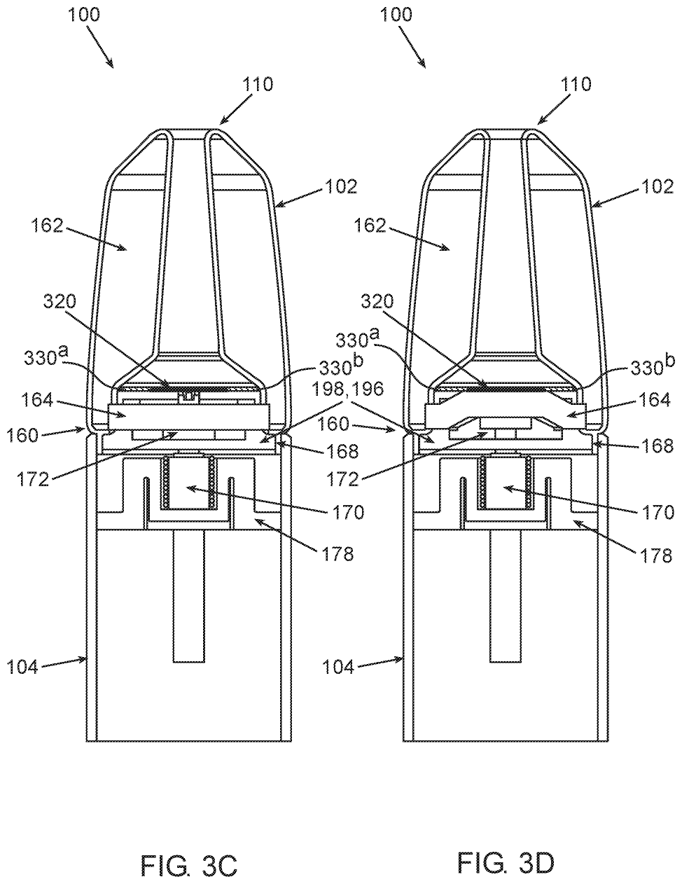

Reference is now made to FIG. 1, FIG. 2 and FIGS. 3A-B. According to some embodiments, liquid deposition mechanism 160 comprises a liquid deposition mechanism housing 178, a liquid container 162, a liquid drawing element 164 and a solenoid mechanism comprising a solenoid actuator 170 connected to a solenoid plunger head 172 through a rod.

FIG. 3A constitutes a cross sectional view of electronic cigarette 100 in the first state of operation and FIG. 3B constitutes a cross sectional view of electronic cigarette 100 in the second state of operation.

Liquid container 162 is accommodated within cartridge internal compartment 108 of cartridge 106 and is configured to contain the liquid therein. In contrast with the discrete volume of liquid, which is small and typically sufficient for a single inhalation of aerosol 166 by a user of 100, liquid container 162 is configured to contain bulk amount of the liquid formulation, wherein only small discrete volume(s) of the liquid are evaporated during the operation of electronic cigarette 100.

According to some embodiments, liquid drawing element 164 is fluidly attached to liquid container 162. According to some embodiments, liquid drawing element 164 is in constant contact with liquid container 162. According to some embodiments, liquid drawing element 164 is partially accommodated within liquid container 162.

According to some embodiments, liquid is provided in liquid container 162 for deliverance towards evaporation heater 120 via liquid drawing element 164.

According to some embodiments, liquid drawing element 164 comprises a material that is capable of incorporating, taking in, drawing in or soaking liquids, and upon applying physical pressure thereto or being in contact with another material, release a portion or the entire amount/volume of the absorbed liquid.

According to some embodiments, liquid drawing element 164 is affixed to at least one of cartridge housing 102, cartridge internal compartment 108 and liquid container 162. According to some embodiments, liquid drawing element 164 is affixed to at least one of cartridge housing 102, cartridge internal compartment 108 and liquid container 162, such that liquid drawing element 164 is in contact with liquid container 162 and capable of withdrawing liquid therefrom.

According to some embodiments, liquid drawing element 164 is flexible, such that upon physical pressure applied on liquid drawing element 164, it is configured to bend, while still being affixed to at least one of cartridge housing 102, cartridge internal compartment 108 and liquid container 162.

According to some embodiments, liquid drawing element 164 is configured to absorb liquid in an amount which is at least 100% of its weight. According to some embodiments, liquid drawing element 164 is configured to absorb liquid in an amount which is at least 50% of its weight.

According to some embodiments, liquid drawing element 164 is fabricated such that contact of liquid drawing element 164 with evaporation heater 120 for said the predetermined period of deposition time results in the delivery of a discrete volume of liquid to evaporation heater 120. According to some embodiments, liquid drawing element 164 is fabricated such that contact of liquid drawing element 164 with evaporation heater 120 for said the predetermined period of deposition time results in the delivery of a thin layer of liquid to evaporation heater 120. According to some embodiments, the thin layer of liquid has thickness in the range of 0.1 mm to 0.5 mm.

According to some embodiments, liquid drawing element 164 comprises cloth, wool, felt, sponge, foam, cellulose, yarn, microfiber or a combination thereof, having high tendency to absorb aqueous solutions. Each possibility represents a separate embodiment. According to some embodiments, the sponge is an open cell sponge. According to some embodiments, the sponge is a closed cell sponge.

According to some embodiments, liquid drawing element 164 comprises fabric. Specifically, fibrous and/or woven fabric, such as a wick, is a hydrophilic and liquid absorbing material, which may be used as the stationary liquid absorbing element(s), according to some embodiments.

According to some embodiments, liquid drawing element 164 is a hydrophilic liquid drawing element. According to some embodiments, liquid drawing element 164 is a hydrophilic sponge.

It is to be understood that electronic cigarette 100 may be used to deliver various aqueous formulation intended for aerosolization, as detailed below. Some of the typical compositions currently used for smoking using paper cigarettes or aerosolization/vaporization using in aerosolization using electronic cigarettes include tobacco products (e.g. nicotine) and cannabis products (e.g. tetrahydrocannabinol—THC, and other cannabinoids, such as cannabidiol—CBD). Nicotine is fairly soluble in aqueous media, enabling in the second state of electronic cigarette 100 for the liquid deposition mechanism 160 to be spaced apart from evaporation heater 120 and deposit liquid thereon from distance. Typical aqueous formulations of cannabis products are not soluble in water, thus requiring a different approach. One approach presented hereinbelow in embodiment directed to the cannabinoid compositions and aerosol compositions, is relating to aqueous compositions for inhalation, which comprises basic salts of THCA (tetrahydrocannabinolic acid) and/or CBDA (cannabidiolic acid). However, this is not the case with typical aqueous formulations of cannabis products, which requires a second approach. Specifically, cannabinoids, which are the biologically active compounds in cannabis, have typically poor aqueous solubility. Therefore, a liquid deposition mechanism, such as liquid deposition mechanism 160 described in FIGS. 1-3, in which liquid drawing element 164 is approaching evaporation heater 120, during the second state of operation of electronic cigarette 100, is required, such that contact is established between evaporation heater 120 and liquid drawing element 164, to enable delivery of the insolubles through direct contact. Moreover, when using liquid deposition mechanism 160 described in FIGS. 1-3, aqueous dispersions (or dispersions including any other solvent) are not required, and slurries or oils of cannabis products, which are highly viscous, may be used as the composition for evaporation.

According to some embodiments, liquid drawing element 164 pressed against evaporation heater 120 in the second state of electronic cigarette 100.

As detailed above, liquid deposition mechanism 160 includes solenoid actuator 170, solenoid plunger head 172 and liquid deposition mechanism housing 178, according to some embodiments. FIG. 2 constitutes a view in which actuator 114 and cartridge 106 are separated, such that none of the elements of liquid deposition mechanism 160 is hidden.

Liquid deposition mechanism housing 178 is located inside actuator 114 and is configured to accommodate solenoid actuator 170. According to some embodiments, liquid deposition mechanism housing 178 is connected to actuator housing 104. According to some embodiments, solenoid actuator 170 is connected to liquid deposition mechanism housing 178.

According to some embodiments, liquid deposition mechanism housing 178 is rigidly attached to actuator housing 104. According to some embodiments, solenoid actuator 170 is attached to liquid deposition mechanism housing 178 such that unintentional displacement of solenoid actuator 170 upwards or downward in the longitudinal direction is prevented. According to some embodiments, liquid deposition mechanism housing 178 is attached to solenoid actuator 170, such that displacement of solenoid actuator 170 upwards or downward in the longitudinal direction is prevented. According to some embodiments, solenoid actuator 170 is attached to liquid deposition mechanism housing 178 such that unintentional displacement of solenoid actuator 170 in a non-longitudinal direction is prevented. According to some embodiments, solenoid actuator 170 is attached to liquid deposition mechanism housing 178, such that displacement of evaporation heater 120 in a non-longitudinal direction is prevented.

Non-longitudinal directions include any direction, which is not along the longitudinal axis, such as any direction orthogonal or angled with respect to the longitudinal axis.

It is to be understood that the restriction of movement enforced on solenoid actuator 170 by liquid deposition mechanism housing 178 refers to restriction of movement of the main body of solenoid actuator 170, but not of its rod or solenoid plunger head 172, which are moving parts, as detailed herein.

According to some embodiments, solenoid actuator 170 is connected to solenoid plunger head 172. According to some embodiments, solenoid actuator 170 is connected to solenoid plunger head 172 through a rod (not numbered).

The term “solenoid” refers to a type of electromagnet, the purpose of which is to generate a controlled magnetic field through a coil wound into a tightly packed helix. The electromagnetic solenoids are used for conversion of electric energy to linear movement. The term “solenoid actuator” as used herein, means one or more electric tubular coils and one or more associated armature members; the coils and members being mounted for relative axial movement with respect to each other.

According to some embodiments, solenoid actuator 170 is configured to receive electric current and to generate axial movements upon receiving the electric current. According to some embodiments, the axial movement of solenoid actuator 170 generates an axial movement of its rod along the along an axis perpendicular to each of evaporation heater 120 and liquid drawing element 164. According to some embodiments, the axial movement of solenoid actuator 170 generates a longitudinal axial movement of its rod. According to some embodiments, upon receiving the electric current solenoid actuator 170 is configured to generate longitudinal movement of its rod at a predetermined rate.

According to some embodiments, processing unit 190 is configured to control solenoid actuator 170. According to some embodiments, processing unit 190 is configured to pass current to solenoid actuator 170. According to some embodiments, upon receiving the electric current solenoid actuator 170 is configured to generate longitudinal movement of its rod at a controlled rate, wherein processing unit 190 is configured to control the controlled rate. According to some embodiments, processing unit 190 is configured to pass variable current to solenoid actuator 170, wherein the variable current is dictating the controlled rate.

The terms “axial” and “axial movement” as used when referring to the operation of solenoid actuator 170 refer to a linear movement along the longitudinal axis of electronic cigarette 100.

According to some embodiments, solenoid plunger head 172 is functionally connected to solenoid actuator 170. According to some embodiments, upon axial movement generated by solenoid actuator 170, solenoid plunger head 172 moves along the longitudinal axis from a first location to a second location. According to some embodiments, the first location is below the second location, as detailed above with respect to directions.

According to some embodiments, in the first state of electronic cigarette 100, solenoid plunger head 172 is in the first location, and both solenoid plunger head 172 and liquid drawing element 164 are spaced apart from evaporation heater 120. According to some embodiments, in the first state of electronic cigarette 100, solenoid plunger head 172 is in the first location, and both solenoid plunger head 172 and liquid drawing element 164 not in contact with evaporation heater 120.

According to some embodiments, in the second state of electronic cigarette 100, solenoid plunger head 172 is in the second location. According to some embodiments, when solenoid plunger head 172 approaches the second location, its pushes a portion of liquid drawing element 164 towards evaporation heater 120. According to some embodiments, in the second state of electronic cigarette 100, solenoid plunger head 172 reaches the second location, and a portion of liquid drawing element 164 contacts evaporation heater 120. According to some embodiments, when liquid drawing element 164 forms contact with evaporation heater 120, delivery of a discrete volume of liquid from liquid drawing element 164 to evaporation heater 120 is enabled.

According to some embodiments, processing unit 190 is configured to alternately operate solenoid actuator 170, such that solenoid plunger head 172 alternately dislocates between the first and second and configured to alternately deliver discrete volumes of liquid to evaporation heater 120.

It will be appreciated that any mechanism, which is configured to move liquid drawing element 164 between a first and a second position is encompassed by the current invention, according to some embodiments. Solenoid mechanisms are widely used for conversion of electric energy to axial movement and are depicted in FIGS. 1-3, however, other mechanisms, which are configured to push liquid drawing element 164 to intermittently contact evaporation heater 120 are also contemplated, according to some embodiments.

According to some embodiments, liquid deposition mechanism 160 is configured to transfer liquid to evaporation heater 120. According to some embodiments, liquid deposition mechanism 160 is configured to deliver a thin film or layer of the liquid to evaporation heater 120. According to some embodiments, liquid deposition mechanism 160 is configured to deliver a film liquid to evaporation heater 120 having a thickness in the range of 0.1 mm to 3 mm. According to some embodiments, the film has a thickness in the range of 0.1 mm to 2 mm. According to some embodiments, the film has a thickness in the range of 0.5 mm to 2 mm. According to some embodiments, the film has a thickness in the range of 0.75 mm to 1.5 mm.

According to some embodiments, liquid deposition mechanism 160 is configured to deliver a discrete volume of liquid to evaporation heater 120, wherein the discrete volume of liquid has a volume in the range of 2 μL to 100 μL. According to some embodiments, the discrete volume of liquid has a volume in the range of 3 μL to 50 μL. According to some embodiments, the discrete volume of liquid has a volume in the range of 4 μL to 45 μL. According to some embodiments, the discrete volume of liquid has a volume in the range of 5 μL to 40 μL. According to some embodiments, the discrete volume of liquid has a volume in the range of 6 μL to 35 μL. According to some embodiments, the discrete volume of liquid has a volume in the range of 7 μL to 30 μL. According to some embodiments, the discrete volume of liquid has a volume in the range of 8 μL to 28 μL. According to some embodiments, the discrete volume of liquid has a volume in the range of 9 μL to 25 μL. According to some embodiments, the discrete volume of liquid has a volume in the range of 10 μL to 20 μL.

According to some embodiments, liquid deposition mechanism 160 is configured to transfer discrete volume of liquid to evaporation heater 120. According to some embodiments, the liquid comprises a nicotine formulation. According to some embodiments, the nicotine formulation is an aqueous nicotine formulation. According to some embodiments, the nicotine formulation is an aqueous nicotine solution. According to some embodiments, the aqueous nicotine formulation comprises from 1% to 5% nicotine w/w. According to some embodiments, the aqueous nicotine formulation comprises from 2% to 4% nicotine w/w. According to some embodiments, the liquid comprises tetrahydrocannabinol (THC). According to some embodiments, the liquid comprises cannabidiol (CBD). According to some embodiments, the liquid comprises cannabis oil. According to some embodiments, the liquid comprises a cannabis slurry. According to some embodiments, liquid container 162 contains the liquid.

According to some embodiments, the liquid comprises a cannabinoid formulation. According to some embodiments, the liquid comprises an aqueous cannabinoid formulation. According to some embodiments, the liquid comprises a basic cannabinoid formulation. According to some embodiments, the aqueous cannabinoid formulation has a pH higher than 7. According to some embodiments, the aqueous cannabinoid formulation has a pH higher than 8. According to some embodiments, the aqueous cannabinoid formulation has a pH higher than 9. According to some embodiments, the aqueous cannabinoid formulation has a pH higher than 10. According to some embodiments, the aqueous cannabinoid formulation has a pH higher than 10.5. According to some embodiments, the aqueous cannabinoid formulation comprises THCA basic salt. According to some embodiments, the aqueous cannabinoid formulation is the cannabinoid composition as presented below.

Prior art liquid deposition mechanisms include liquid containers, which are in constant contact with the respective heater, or which deliver liquid constantly during the electronic cigarette operation through a delivery medium (typically a wick positioned in stationary contact with both the liquid container and the heater). In contrast, liquid deposition mechanism 160 of electronic cigarette 100 includes liquid container 162, which is configured to deliver liquid to liquid drawing element 164 constantly, but liquid drawing element 164 is separated from evaporation heater 120 in the first state of electronic cigarette 100, such that delivery of liquid is not constant.

According to some embodiments, liquid deposition mechanism 160, or parts thereof is configured to be in transient contact with evaporation heater 120, such that discrete amounts of the liquid are intermittently delivered from liquid deposition mechanism 160 to evaporation heater 120.

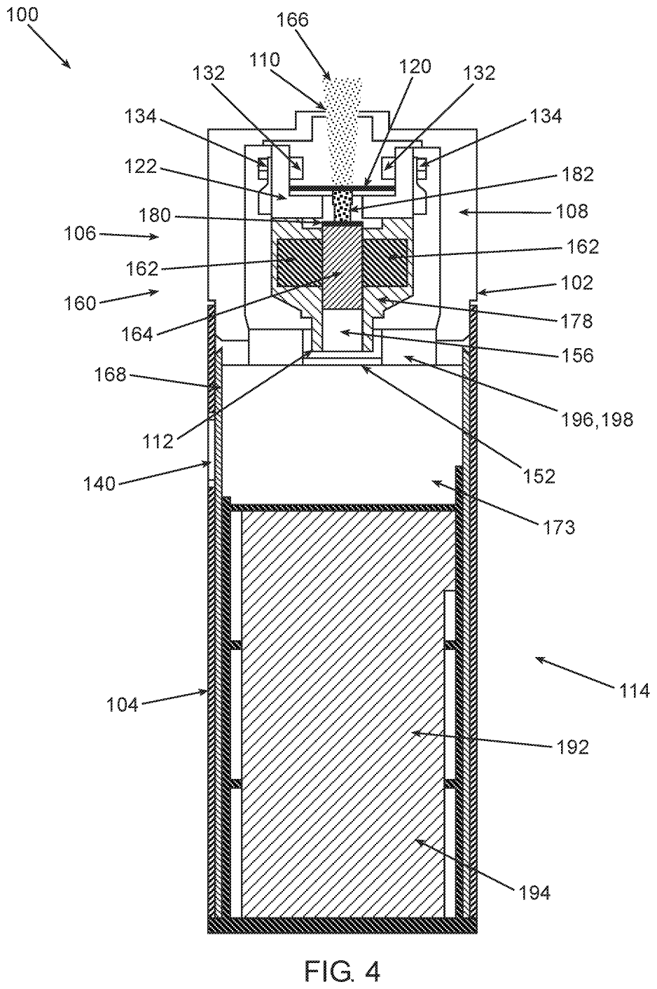

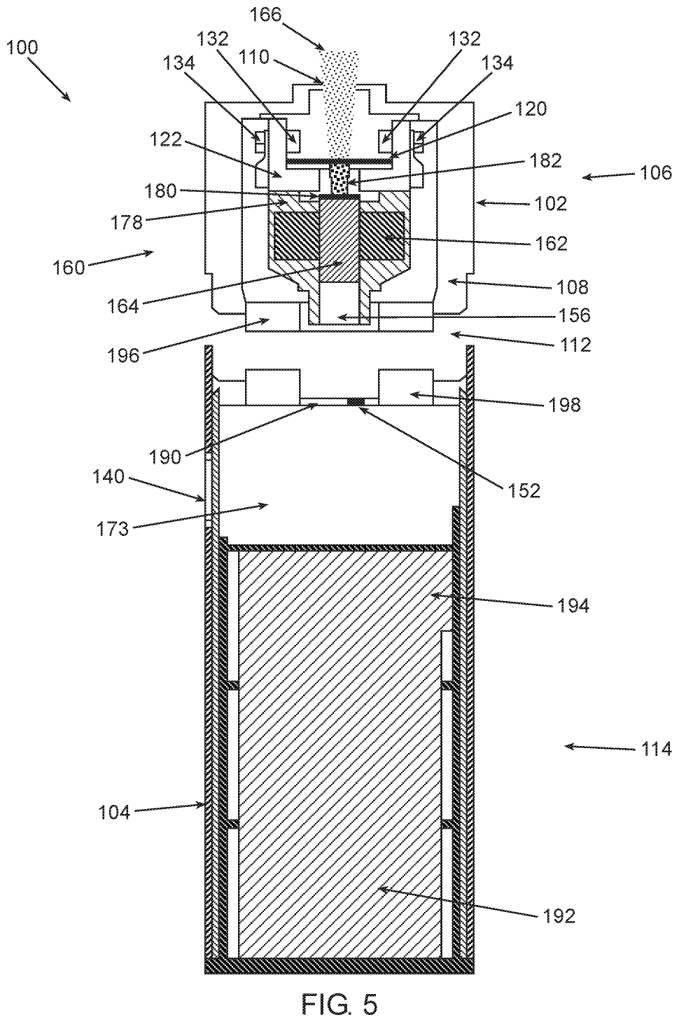

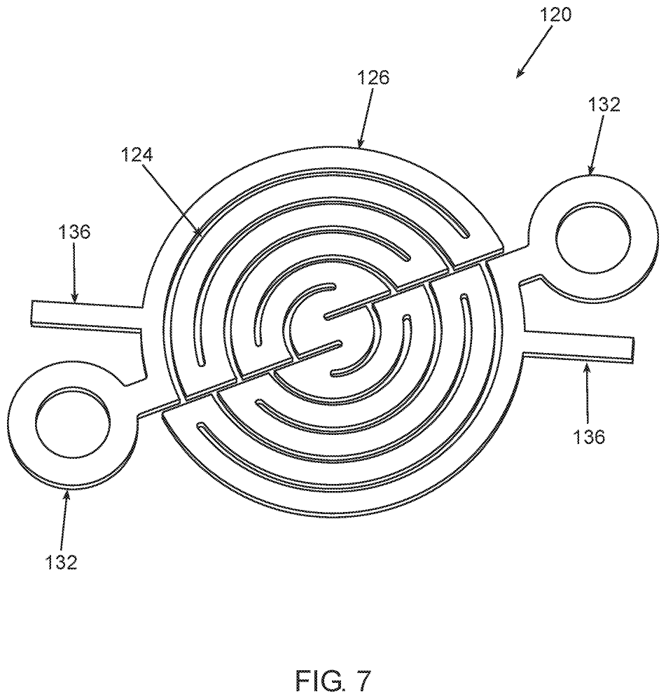

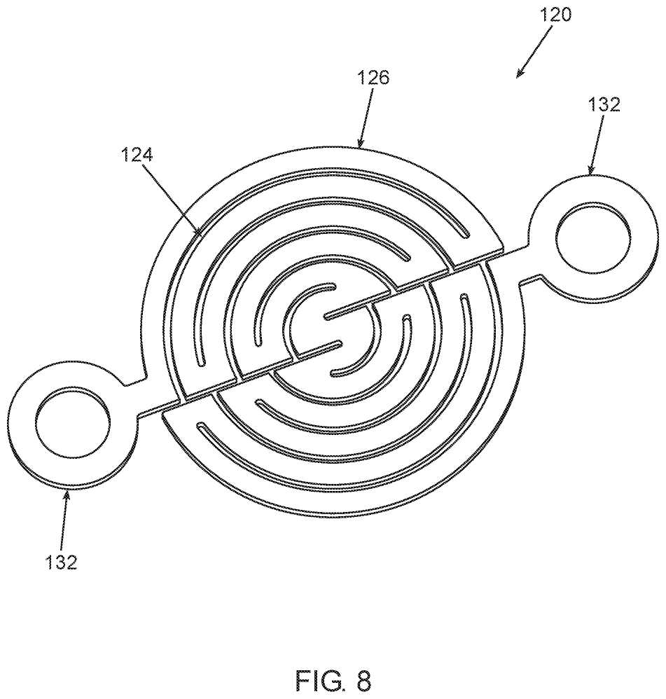

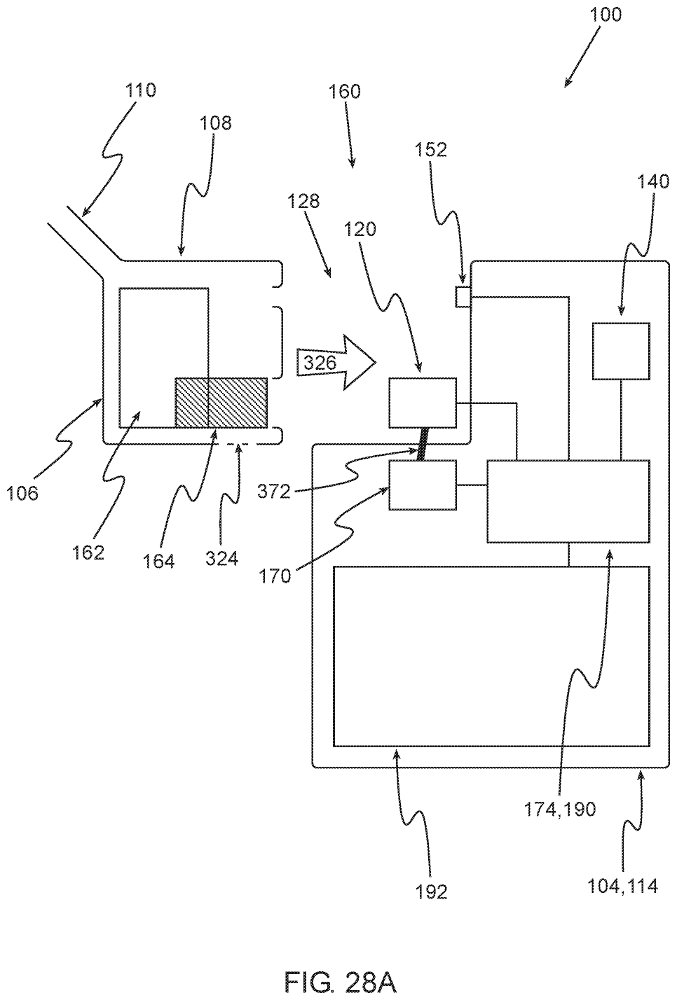

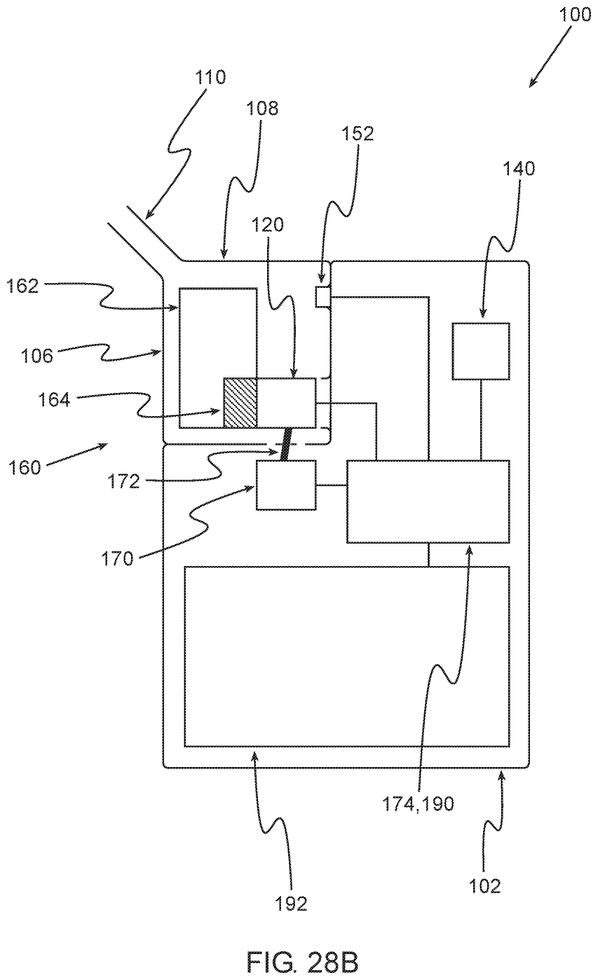

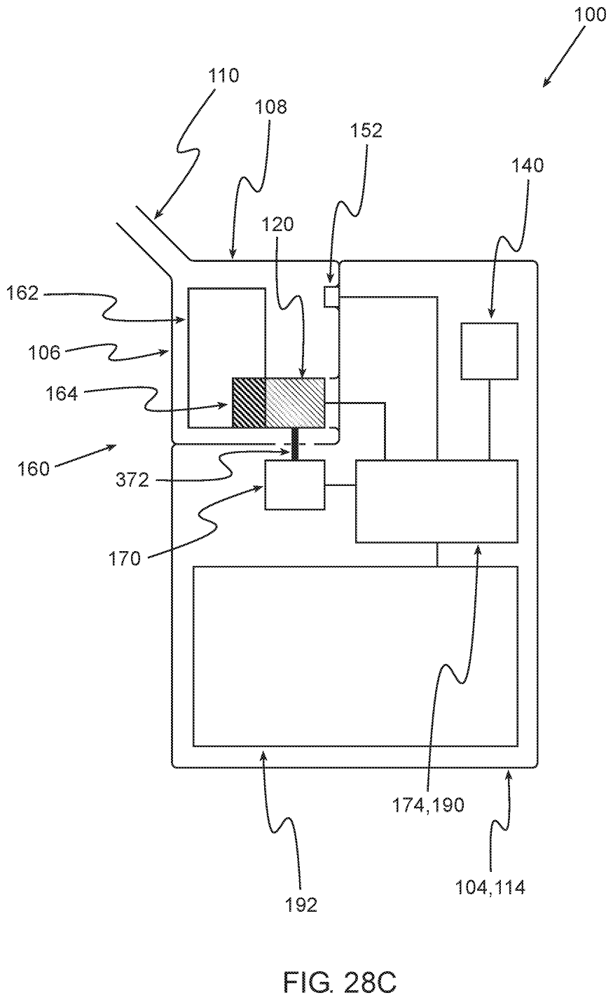

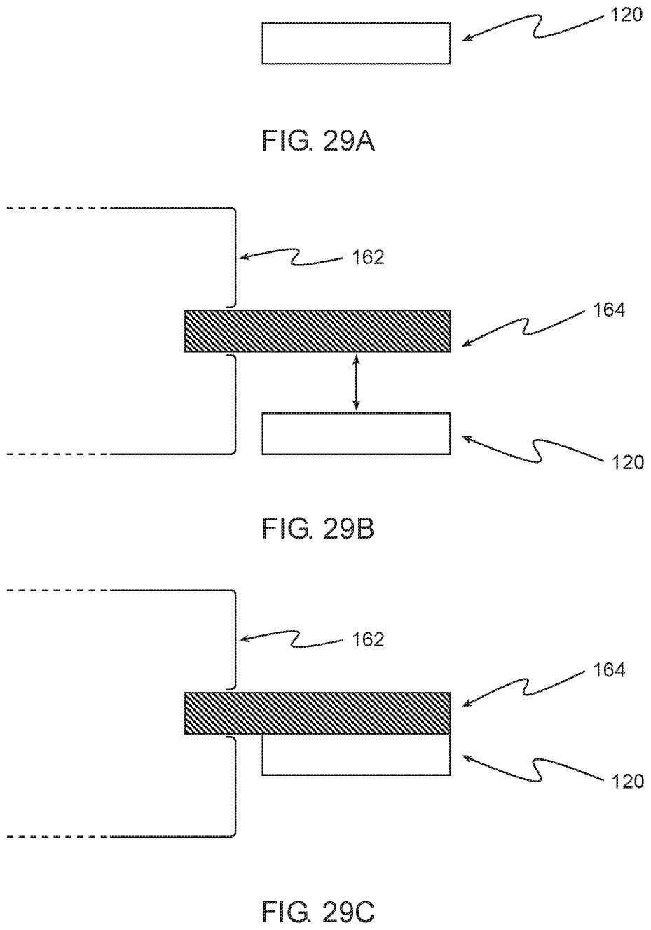

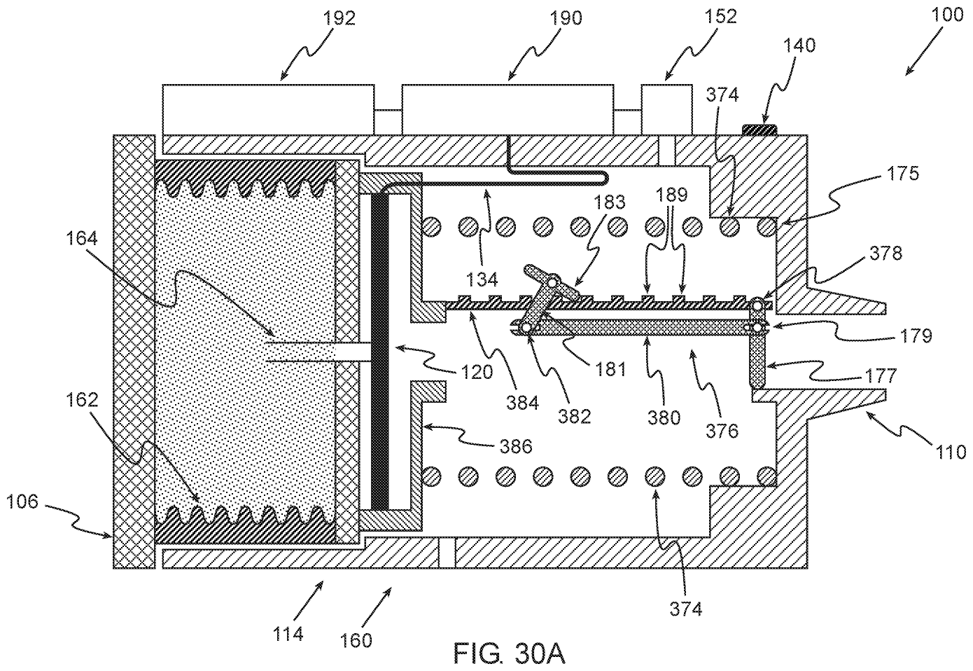

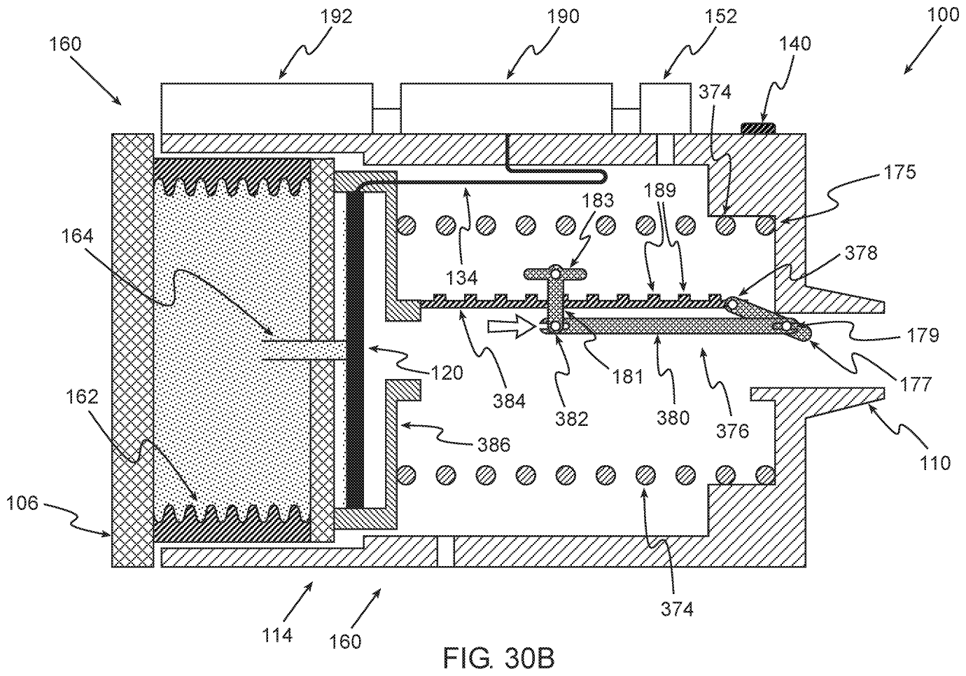

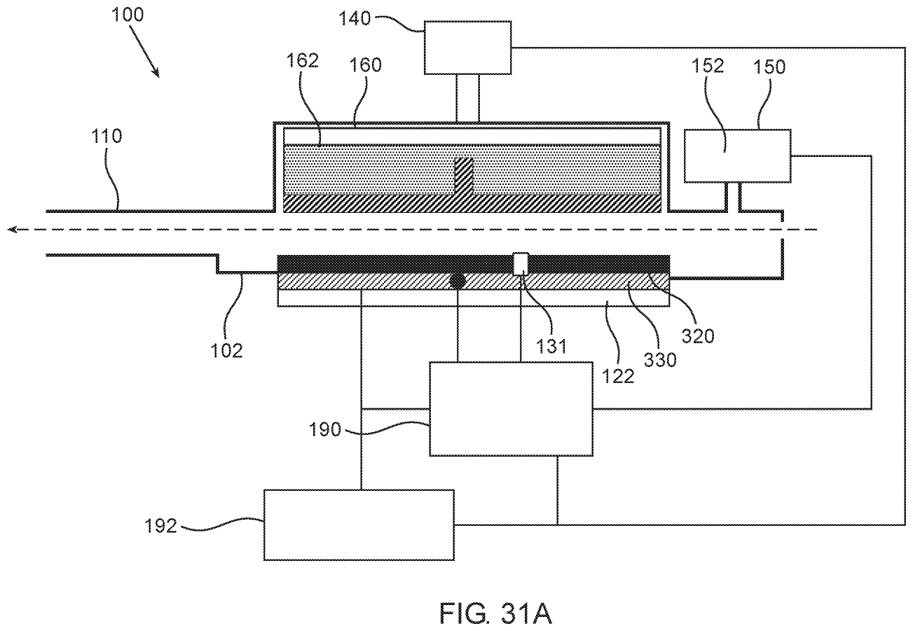

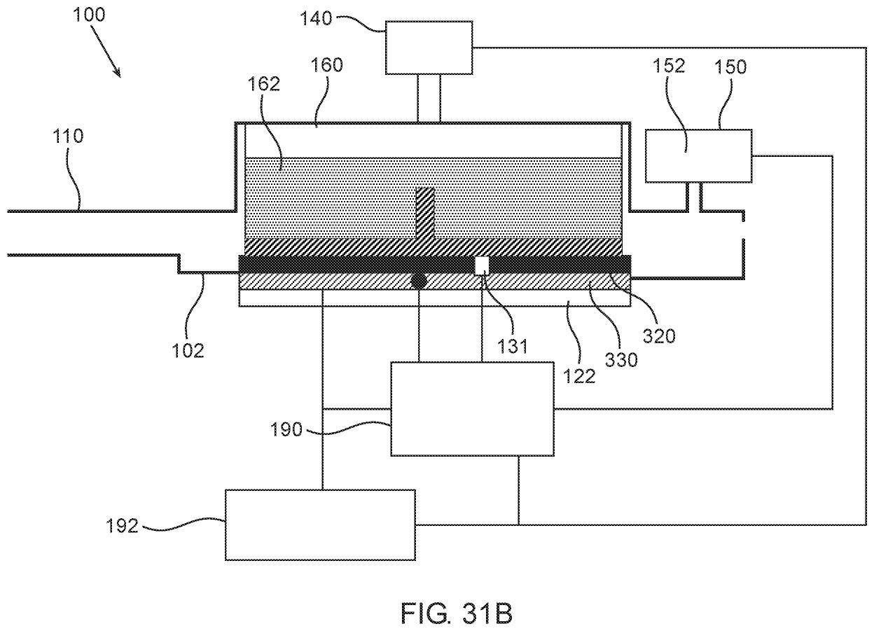

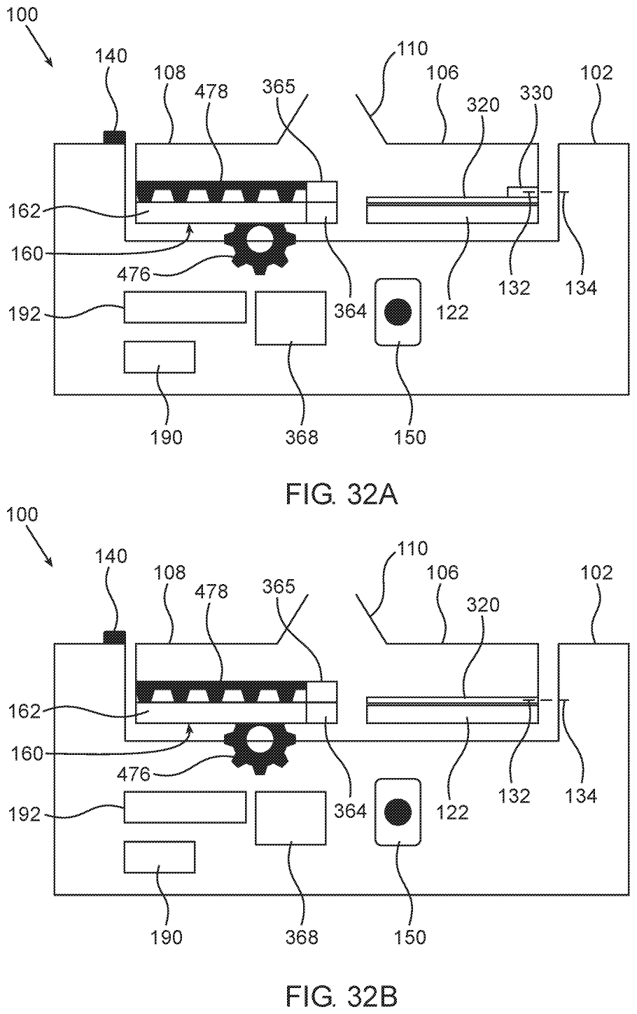

Reference is now made to FIGS. 7-11. It is to be understood that embodiments referring to evaporation heater 120 and FIGS. 7-11 apply to any electronic cigarettes 100 as presented herein. Specifically, embodiments referring to evaporation heater 120 and FIGS. 7-11 apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

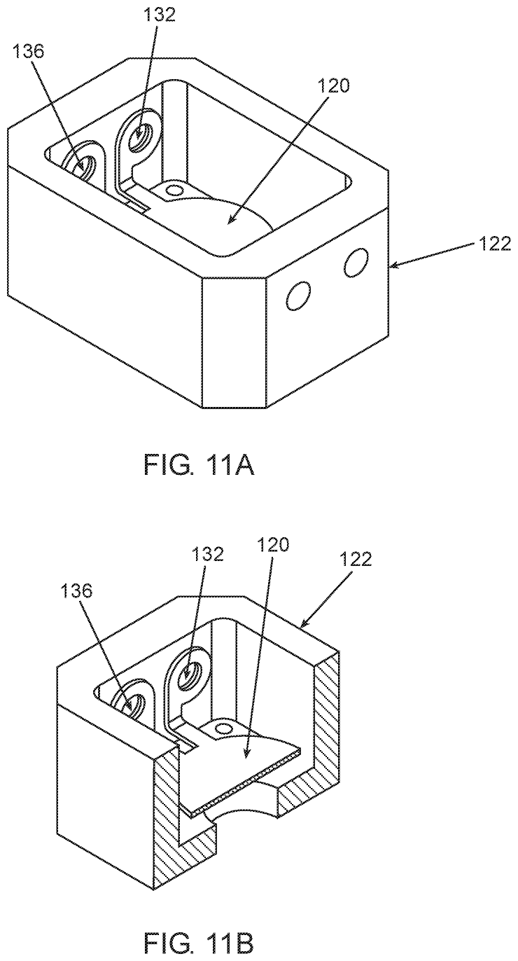

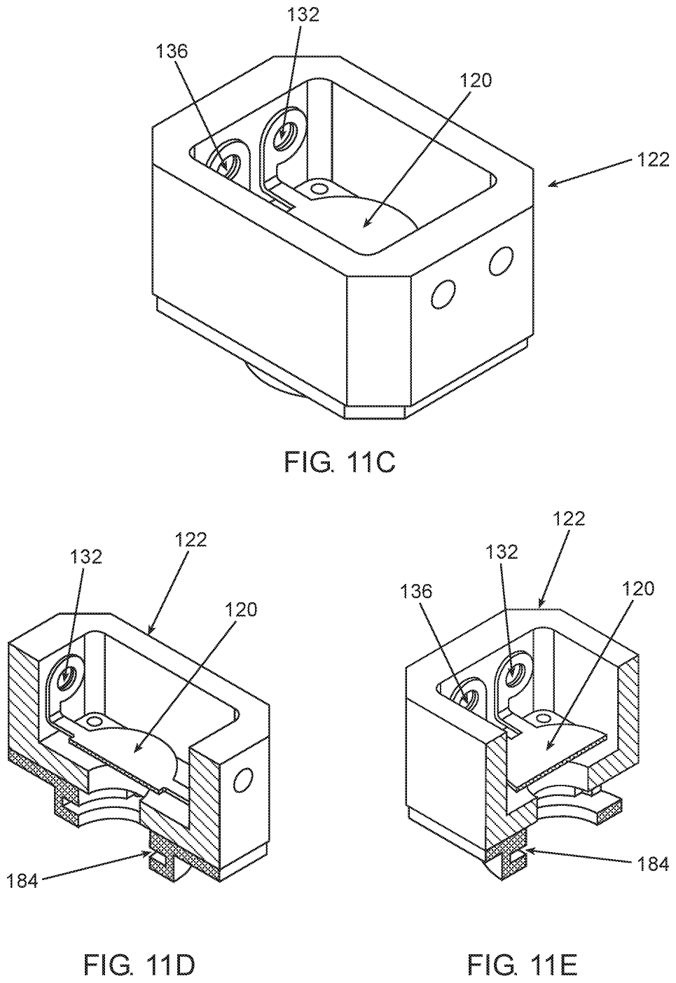

FIGS. 7-10 constitute views of evaporation heater 120, when separated from electronic cigarette 100, and FIG. 11 constitutes views of evaporation heater 120, housed within support 122 when separated from electronic cigarette 100.

According to some embodiments, electronic cigarette 100 further comprises a support 122. According to some embodiments, support 122 is rigidly attached to cartridge housing 102. According to some embodiments, evaporation heater 120 is attached to support 122 such that unintentional displacement of evaporation heater 120 upwards or downward in the longitudinal direction is prevented. According to some embodiments, evaporation heater 120 is attached to support 122 such that displacement of evaporation heater 120 upwards or downward in the longitudinal direction is prevented. According to some embodiments, evaporation heater 120 is attached to support 122 such that unintentional displacement of evaporation heater 120 in a non-longitudinal direction is prevented. According to some embodiments, evaporation heater 120 is attached to support 122 such that displacement of evaporation heater 120 in a non-longitudinal direction is prevented.

According to some embodiments, support 122 comprises a high-temperature resistant with low thermal conductivity material, such as conventional ceramics or aluminum silicate ceramics, titanium oxide, zirconium oxide, yttrium oxide ceramics, molten silicon, silicon dioxide and molten aluminum oxide. According to some embodiments, support 122 is made of ceramics.

According to some embodiments, evaporation heater 120 is configured to function both as an evaporation element and as a heating element. According to some embodiments, processing unit 190 is configured to control the operation of evaporation heater 120 and to regulate its temperature.

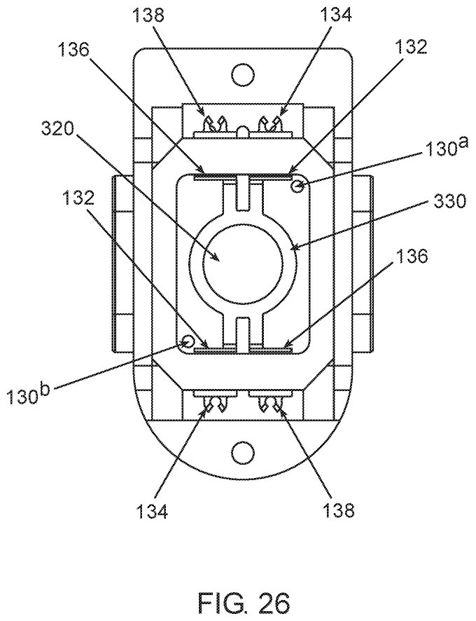

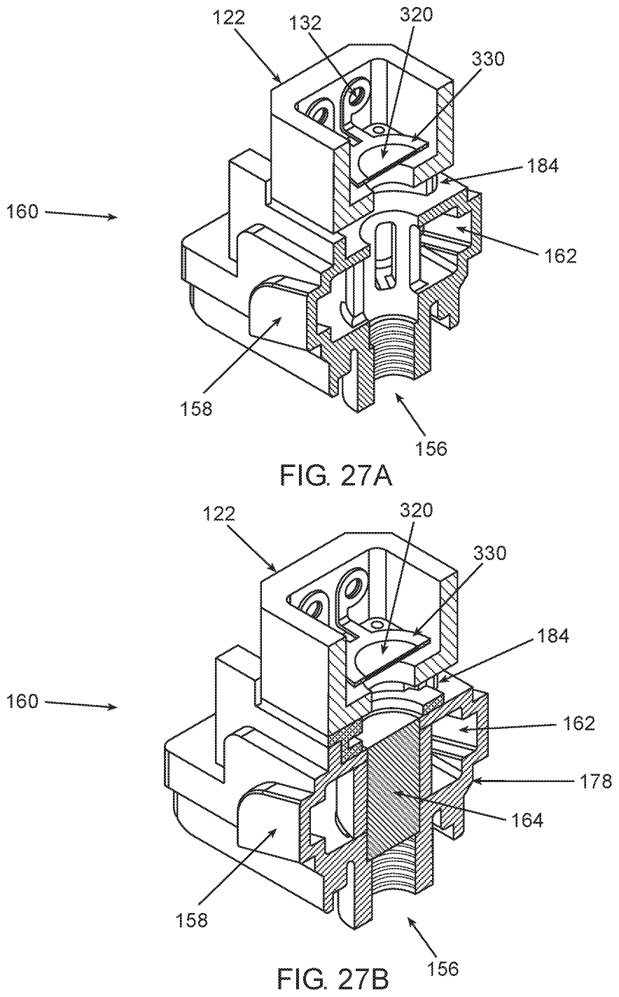

Advantageously, incorporating the both the heating functionality and the evaporating surface functionality within a single element, as in evaporation heater 120 reduces the number of components within electronic cigarette 100, thereby potentially reducing both space and costs. However, evaporation heater 120 may include, separately, a heater element and an evaporation medium element, according to some embodiments. Specific embodiments directed to separate heater element and an evaporation medium element are further discussed e.g. when referring to FIGS. 3C, 3D, 25-27, 31A, 31B and 32A.

According to some embodiments, evaporation heater 120 is a unitary element configured both to provide an evaporation surface and to generate heat.

According to some embodiments, evaporation heater 120 is housed within cartridge housing 102.

Without wishing to be bound by any theory or mechanism of action, upon heating of evaporation heater 120 the liquid is at least partially vaporized into vapor. Subsequently, the vapor in condensed into aerosol, which may be inhaled by a user in need thereof, such as an electronic cigarette user.

According to some embodiments, evaporation heater 120 is rigid. According to some embodiments, evaporation heater 120 is made of metal. According to some embodiments, evaporation heater 120 has two flat sides, which remain flat when liquid is pressed there through. According to some embodiments, evaporation heater 120 has a top flat surface and a bottom flat surface, which do not deform when liquid is pressed there through or pressed against at least one of the top surface or the bottom surface.

According to some embodiments, evaporation heater 120 is configured to provide 3-7 W, 4-6 W, 4.5-5.9 W, 4.8-5.6 W, 5.0-5.4 W or 5.1-5.3 W per every μl of liquid deposited thereon.

According to some embodiments, evaporation heater 120 is configured to provide about 5.2 W per every μl of liquid deposited thereon.

According to some embodiments evaporation heater 120 has heat capacity of no more than 1000 Jkg⁻¹K⁻¹. According to some embodiments, evaporation heater 120 has heat capacity of no more than 900 Jkg⁻¹K⁻¹. According to some embodiments, evaporation heater 120 has heat capacity of no more than 800 Jkg⁻¹K⁻¹. According to some embodiments, evaporation heater 120 has heat capacity of no more than 700 Jkg⁻¹K⁻¹. According to some embodiments, evaporation heater 120 has heat capacity of no more than 600 Jkg⁻¹K⁻¹.

According to some embodiments, evaporation heater 120 has a specific heat capacity in the range of 100 to 900 Jkg⁻¹K⁻¹. According to some embodiments, evaporation heater 120 has a specific heat capacity in the range of 200 to 800 Jkg⁻¹K⁻¹. According to some embodiments, evaporation heater 120 has a specific heat capacity in the range of 300 to 750 Jkg⁻¹K⁻¹. According to some embodiments, evaporation heater 120 has a specific heat capacity in the range of 400 to 700 Jkg⁻¹K⁻¹. According to some embodiments, evaporation heater 120 has a specific heat capacity in the range of 450 to 650 Jkg⁻¹K⁻¹. According to some embodiments, evaporation heater 120 has a specific heat capacity in the range of 500 to 600 Jkg⁻¹K⁻¹. According to some embodiments, evaporation heater 120 has a specific heat capacity in the range of 470 to 570 Jkg⁻¹K⁻¹. According to some embodiments, evaporation heater 120 has a specific heat capacity in the range of 485 to 555 Jkg⁻¹K⁻¹. According to some embodiments, evaporation heater 120 has a specific heat capacity in the range of 500 to 540 Jkg⁻¹K⁻¹.

According to some embodiments, evaporation heater 120 has surface heat flux in the range of 170 Wcm⁻² to 290 Wcm⁻². According to some embodiments, evaporation heater 120 has surface heat flux in the range of 200 Wcm⁻² to 260 Wcm⁻². According to some embodiments, evaporation heater 120 has surface heat flux in the range of 210 Wcm⁻² to 250 Wcm⁻². According to some embodiments, evaporation heater 120 has surface heat flux in the range of 220 Wcm⁻² to 240 Wcm⁻². According to some embodiments, evaporation heater 120 has surface heat flux of about 228 Wcm⁻².

According to some embodiments, evaporation heater 120 is configured to provide an energy output of at least 35 Joules within half a second. According to some embodiments, evaporation heater 120 is configured to provide an energy output of at least 40 Joules within half a second. According to some embodiments, evaporation heater 120 is configured to provide an energy output of at least 45 Joules within half a second. According to some embodiments, evaporation heater 120 is configured to provide an energy output of at least 50 Joules within half a second. According to some embodiments, evaporation heater 120 is configured to provide an energy output of at least 51 Joules within half a second.

According to some embodiments, evaporation heater 120 has a total resistance in the range of 0.10Ω to 0.60Ω. According to some embodiments, evaporation heater 120 has a total resistance in the range of 0.13Ω to 0.55Ω. According to some embodiments, evaporation heater 120 has a total resistance in the range of 0.15Ω to 0.5Ω. According to some embodiments, evaporation heater 120 has a total resistance in the range of 0.15Ω to 0.45Ω. According to some embodiments, evaporation heater 120 has a total resistance in the range of 0.2Ω to 0.4Ω.

According to some embodiments, evaporation heater 120 is configured to provide an energy output of at least 30 Watts. According to some embodiments, evaporation heater 120 is configured to provide an energy output of at least 40 Watts. According to some embodiments, evaporation heater 120 is configured to provide an energy output of at least 5. According to some embodiments, evaporation heater 120 is configured to provide an energy output of at least 60 Watts. According to some embodiments, evaporation heater 120 is configured to provide an energy output of at least 70 Watts. According to some embodiments, evaporation heater 120 is configured to provide an energy output of at least 80 Watts. According to some embodiments, evaporation heater 120 is configured to provide an energy output of at least 90 Watts. According to some embodiments, evaporation heater 120 is configured to provide an energy output of at least 100 Watts. According to some embodiments, evaporation heater 120 is configured to provide an energy output of at least 102 Watts.

According to some embodiments, evaporation heater 120 is configured to drive current in the range of 10 A and 40 A. According to some embodiments, evaporation heater 120 is configured to drive current in the range of 15 A and 35 A. According to some embodiments, evaporation heater 120 is configured to drive current in the range of 20 A and 30 A. According to some embodiments, evaporation heater 120 is configured to drive current in the range of 25 A and 30 A. According to some embodiments, evaporation heater 120 is configured to drive current of about 28 A.

According to some embodiments, evaporation heater 120 is disposable. According to some embodiments, evaporation heater 120 is in the form of a rod, a capsule or a flat disc.

According to some embodiments, evaporation heater 120 comprises a thermally-conductive material, such as metal.

According to some embodiments, evaporation heater 120 has thermal mass of not more than 0.3 J/C. According to some embodiments, evaporation heater 120 has thermal mass of not more than 0.2 J/C. According to some embodiments, evaporation heater 120 has thermal mass of not more than 0.1 J/C. According to some embodiments, evaporation heater 120 has thermal mass of less than 0.1 J/C. According to some embodiments, evaporation heater 120 has thermal mass of less than 0.08 J/C. According to some embodiments, evaporation heater 120 has thermal mass of less than 0.06 J/C. According to some embodiments, evaporation heater 120 has thermal mass of less than 0.04 J/C. According to some embodiments, evaporation heater 120 has thermal mass of less than 0.3 J/C. According to some embodiments, evaporation heater 120 has thermal mass of less than 0.2 J/C.

According to some embodiments, evaporation heater 120 has thermal mass in the range of 0.001 J/C to 0.3 J/C. According to some embodiments, evaporation heater 120 has thermal mass in the range of 0.004 J/C to 0.25 J/C. According to some embodiments, evaporation heater 120 has thermal mass in the range of 0.006 J/C to 0.2 J/C. According to some embodiments, evaporation heater 120 has thermal mass in the range of 0.01 J/C to 0.015 J/C.

According to some embodiments, evaporation heater 120 is made of a uniform material. According to some embodiments, evaporation heater 120 is made of metal. According to some embodiments, evaporation heater 120 comprises a metal and/or a metal alloy. According to some embodiments, evaporation heater 120 comprises a metal alloy. According to some embodiments, evaporation heater 120 comprises at least one metal selected from iron, nickel, titanium, chromium, aluminum, molybdenum and manganese. According to some embodiments, the alloy comprises at least one metal selected from iron, nickel, titanium, chromium, aluminum, molybdenum, silver, palladium and manganese. Each possibility represents a separate embodiment. According to some embodiments, evaporation heater 120 comprises a metal having electrical resistivity in the range of 0.3·10⁻⁶ to 3·10⁻⁶ Ω·m at room temperature. According to some embodiments, evaporation heater 120 comprises a metal having electrical resistivity in the range of 0.4·10⁻⁶ to 2.5·10⁻⁶ Ω·m at room temperature. According to some embodiments, evaporation heater 120 comprises a metal having electrical resistivity in the range of 0.5·10⁻⁶ to 2·10⁻⁶ Ω·m at room temperature. According to some embodiments, evaporation heater 120 comprises a metal having electrical resistivity in the range of 0.6·10⁻⁶ to 1.5·10⁻⁶ Ω·m at room temperature. According to some embodiments, evaporation heater 120 comprises an alloy having electrical resistivity in the range of 0.3·10⁻⁶ to 3·10⁻⁶ Ω·m at room temperature. According to some embodiments, evaporation heater 120 comprises an alloy having electrical resistivity in the range of 0.4·10⁻⁶ to 2.5·10⁻⁶ Ω·m at room temperature. According to some embodiments, evaporation heater 120 comprises an alloy having electrical resistivity in the range of 0.5·10⁻⁶ to 2·10⁻⁶ Ω·m at room temperature. According to some embodiments, evaporation heater 120 comprises an alloy having electrical resistivity in the range of 0.6·10⁻⁶ to 1.5·10⁻⁶ Ω·m at room temperature. According to some embodiments, the alloy is selected from Kanthal, Nichrome and stainless steel. According to some embodiments, the alloy is Nichrome. According to some embodiments, the alloy is stainless steel. According to some embodiments, the alloy is 316L stainless steel.

According to some embodiments, evaporation heater 120 is configured to generate heat rapidly, such that its temperature elevate rapidly.

According to some embodiments, evaporation heater 120 is configured to generate sufficient heat so as to elevate its temperature to a value high enough to at least partially evaporate the liquid contained by or in direct contact with evaporation heater 120, thereby enabling electronic cigarette 100 to produce vapor comprising a constant and reproducible dose. According to some embodiments, evaporation heater 120 is configured to generate sufficient heat so as to elevate its temperature to a value high enough to at least partially evaporate water contained by or in direct contact with evaporation heater 120, thereby enabling electronic cigarette 100 to produce water vapor comprising a constant and reproducible dose. According to some embodiments, evaporation heater 120 is configured to generate sufficient heat so as to elevate its temperature to a value high enough to at least partially vaporize nicotine contained by or in direct contact with evaporation heater 120, thereby enabling electronic cigarette 100 to produce nicotine vapor comprising a constant and reproducible dose. According to some embodiments, evaporation heater 120 is configured to generate sufficient heat so as to elevate its temperature to a value high enough to at least partially vaporize a cannabinoid contained by or in direct contact with evaporation heater 120, thereby enabling electronic cigarette 100 to produce cannabinoid vapor comprising a constant and reproducible dose. According to some embodiments, evaporation heater 120 is configured to generate sufficient heat so as to elevate its temperature to a value high enough to substantially evaporate the liquid contained by or in direct contact with evaporation heater 120, thereby enabling electronic cigarette 100 to produce vapor comprising a constant and reproducible dose. According to some embodiments, evaporation heater 120 is configured to generate sufficient heat so as to elevate its temperature to a value high enough to substantially evaporate water contained by or in direct contact with evaporation heater 120, thereby enabling electronic cigarette 100 to produce water vapor comprising a constant and reproducible dose. According to some embodiments, evaporation heater 120 is configured to generate sufficient heat to so as to elevate its temperature to a value high enough to substantially evaporate nicotine contained by or in direct contact with evaporation heater 120, thereby enabling electronic cigarette 100 to produce nicotine vapor comprising a constant and reproducible dose. According to some embodiments, evaporation heater 120 is configured to generate sufficient heat to so as to elevate its temperature to a value high enough to substantially evaporate a cannabinoid contained by or in direct contact with evaporation heater 120, thereby enabling electronic cigarette 100 to produce cannabinoid vapor comprising a constant and reproducible dose.

According to some embodiments, evaporation heater 120 is configured to generate heat, to reach a temperature in the range between 50 and 600 degrees Celsius. According to some embodiments, the temperature is at least 95° C., at least 96° C., at least 97° C., at least 98° C., at least 98.5° C., at least 99° C., at least 99.5° C., or at least 100° C. According to some embodiments, the temperature is not more than 600° C., not more than 550° C., not more than 500° C., not more than 450° C., not more than 400° C., not more than 350° C. or not more than 300° C.

According to some embodiments, evaporation heater 120 comprises a resistive heater. According to some embodiments, evaporation heater 120 comprises a radio-frequency heater. According to some embodiments, evaporation heater 120 comprises an induction-coil heater.

According to some embodiments, evaporation heater 120 is in direct contact with evaporation heater 120, to conduct heat thereto.

Without wishing to be bound by any theory or mechanism of action, evaporation heater 120 is required to include a relatively strong heater. Specifically, electronic cigarette 100 is designed to evaporate aqueous compositions, according to some embodiments. Thus use of water, however, may pose several obstacles. Importantly, water has a high latent heat value, meaning that substantial energy has to be invested in order to evaporate water. Thus, according to some embodiments, evaporation heater 120 is a strong heater configured to generate enough heat to vaporize an aqueous solution of nicotine (2-5%) at a rate of at least 0.1 mg nicotine per second, which is considered to provide satisfying consumer experience. In addition, according to some embodiments, evaporation heater 120 is a strong heater configured to generate enough heat to vaporize an aqueous solution of cannabinoid(s) (e.g. the cannabinoid composition disclosed herein below, having cannabinoid concentration of 3-7%) at a rate of at least 0.25 mg THC, THCA or a salt thereof per second, which is considered to provide satisfying consumer experience. According to some embodiments, evaporation heater 120 is configured to generate at least 30 W power. According to some embodiments, evaporation heater 120 is configured to generate at least 32 W power. According to some embodiments, evaporation heater 120 is configured to generate at least 34 W power. According to some embodiments, evaporation heater 120 is configured to generate at least 36 W power. According to some embodiments, evaporation heater 120 is configured to generate at least 8 W power. According to some embodiments, evaporation heater 120 is configured to generate at least 40 W power.

An additional obstacle encountered when dealing with evaporation aqueous composition, is the slow evaporation thereof, which stems from the high specific heat capacity of water as well as from the high latent heat of water. Both these high values entail investment of a substantial amount of energy, which in turn, is slower than when using organic formulations (i.e. PG or VG). Thus, together with high electrical power, an additional requirement from evaporation heater 120 is directed to it low thermal mass. According to some embodiments, evaporation heater 120 has thermal mass of less than 0.05 J/C.

According to some embodiments, evaporation heater 120 is a strong heater configured to generate enough heat to vaporize an aqueous solution of nicotine (2-5%) at a rate of at least 0.4 mg nicotine per second, which is considered to provide satisfying consumer experience.

According to some embodiments, evaporation heater 120 is a strong heater configured to generate enough heat to vaporize an aqueous solution of nicotine (2-5%) at a rate of at least 0.25 mg tetrahydrocannabinol per second, which is considered to provide satisfying consumer experience.

According to some embodiments, processing unit 190 is configured to receive at least one operation signal and to control operation of evaporation heater 120 upon receiving the at least one operation signal. According to some embodiments, processing unit 190 is configured to regulate the temperature of evaporation heater 120. According to some embodiments, the regulation entails maintaining the temperature of evaporation heater 120 in the range of 95° C. to 400° C. Preferably, the temperature of evaporation heater 120 is maintained in the range of 99.5° C. to 350° C. According to some embodiments, processing unit 190 is configured to control operation of evaporation heater 120, which generates heat and elevates its temperature, thereby regulating the temperature of evaporation heater 120.

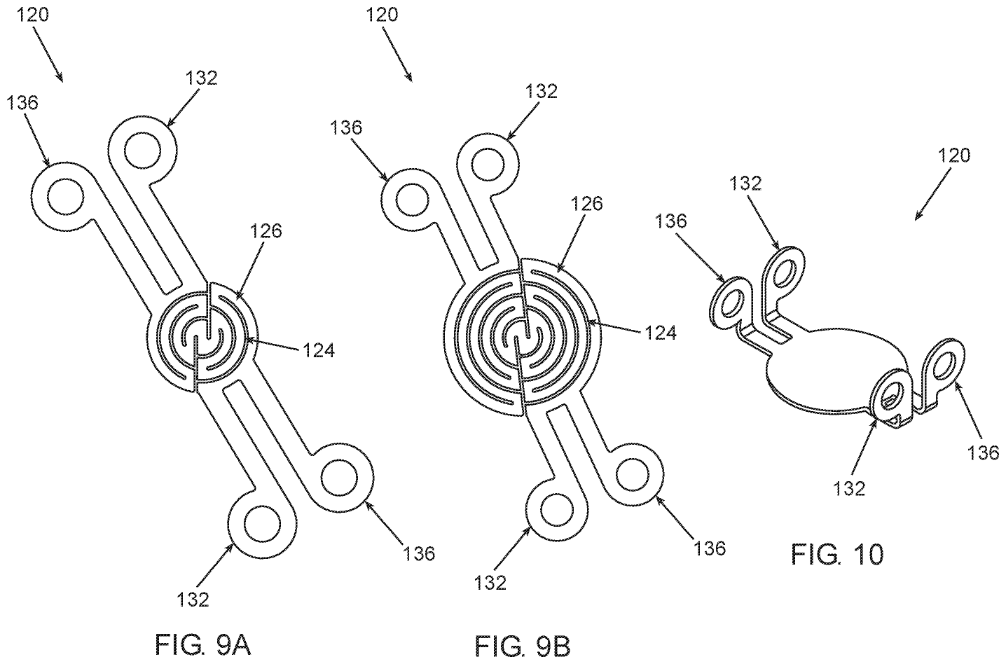

As shown in FIGS. 7 and 9, evaporation heater 120 includes an elongated heat conductive coil 126, which is formed in a two dimensional shape. Although evaporation heater 120 is shown as circular, any shape, formed by elongated heat conductive coil 126 is contemplated according to some embodiments.

According to some embodiments, elongated heat conductive coil 126 is welded in each of its two ends to an evaporation heater electric contact 132. According to some embodiments, elongated heat conductive coil 126 is formed in a structure, in which current driving from between evaporation heater electric contacts 132 is driving throughout its length. Specifically, elongated heat conductive coil 126 is a substantially 1-dimentional coil bent up to form a 2-dimensional structure, wherein the elongated heat conductive coil 126 is uninterrupted throughout its length by internal contact with linearly non-consecutive portions of its length. The term “substantially 1-dimensional” refers to an object having three dimensions, wherein the largest dimension is at least 10 times longer than each of the other two dimensions.

Thus, as shown in FIGS. 7-9 elongated heat conductive coil 126 is shaped elongating along a meandering path, according to some embodiments. According to some embodiments, the meandering path forms a 2-D structure. According to some embodiments, each of the two ends of elongated heat conductive coil 126 is connected to an evaporation heater electric contact 132. According to some embodiments, each of evaporation heater electric contacts 132 is positioned on the circumference of the 2-D structure formed by elongated heat conductive coil 126 shaping. The curved meandering path of elongated heat conductive coil 126 includes turns, which form inner tracks 124 between curves formed by the turns, according to some embodiments. This shaping ensures that current is driven between evaporation heater electric contacts 132 with a maximal length (i.e. maximal resistive length), according to some embodiments. The longer the resistive length, the higher the resistance of the respective evaporation heater 120. For example, if all the other variables are maintained constant, evaporation heater 120 in FIG. 9B will have higher resistance than evaporation heater 120 of FIG. 9A.

The structure of evaporation heater 120 and elongated heat conductive coil 126 ensures that the resistive length of evaporation heater 120 is high, and therefore its resistance is high and it may produce heat quickly.

According to some embodiments, inner tracks 124 are formed between the length and bends of elongated heat conductive coil 126. According to some embodiments, inner tracks 124 enable the passage of material (e.g. fluids) through evaporation heater 120. Specifically, as shown is FIGS. 1-5, the liquid formulation from liquid deposition mechanism 160 is configured to contact evaporation heater 120 at a bottom surface thereof, and vapor is released from a top surface of evaporation heater 120, according to some embodiments. This is enabled, according to some embodiments, since the evaporating material passes through inner tracks 124.

It was surprisingly found that the formation of evaporation heater 120 with inner tracks 124 results in the reduction or elimination of the Leidenfrost effect, which is associated with aqueous formulations, according to some embodiments. Thus, an additional beneficial feature of inner tracks 124 is that it enables the prevention of the Leidenfrost effect. Specifically, inner tracks 124 act as capillary channel, which reduce Leidenfrost effect using a capillary effect, which is not enabled by standard flat impervious heaters or evaporation media connected to heaters.

It is an additional beneficial feature of inner tracks 124 that the enable the discrete volume of liquid deposited on evaporation heater 120 to cling on to the bottom surface of evaporation heater 120. Specifically, upon deposition of the discrete volume of liquid on a flat uniform/impermeable heated surface, portions of the discrete volume of liquid may ‘bounce’ off the bottom surface downwards, whereas the capillary structure of elongated heat conductive coil 126 and inner tracks 124 prevents or reduces the bounce-off effect. The capillary tracks are also very important in “pinning the liquid” to the evaporation surface especially when using the piezo option. Without these tracks, liquid ejected by the piezo would bounce off the heater.

In order to further improve the understanding of the present disclosure additional information regarding the Leidenfrost effect is given below. The Leidenfrost effect is a phenomenon in which a liquid, in near contact with a surface significantly hotter than the liquid's boiling temperature, produces an insulating vapor layer which keeps that liquid from boiling rapidly. U.S. Pat. No. 6,450,183 provides a thorough explanation of the Leidenfrost effect.

The term “Leidenfrost temperature”, as used herein, refers to the temperature threshold at which the Leidenfrost effect occurs at given conditions. In some embodiments, the Leidenfrost temperature is determined for a pair of solid and liquid materials. For example, for a saturated water-copper interface, the Leidenfrost temperature is 257° C.

The Leidenfrost temperatures for glycerol and other common alcohols and glycols are significantly smaller because of the lower surface tension values and higher viscosities of these solvents. As a result, “e-juice” compositions are typically based on alcohols, rather than on aqueous solutions/emulsions, despite the health hazard associated with alcohol burning.

With reference to FIG. 10, evaporation heater electric contacts 132 are bent with respect to the surfaces of evaporation heater 120. As shown in FIGS. 11A and 11B evaporation heater electric contact 132 pass through support 122 to achieve an electric contact with matching cartridge electric contacts 134, according to some embodiments. According to some embodiments, cartridge electric contacts 134 are formed as crimp ring terminals. According to some embodiments, cartridge electric contacts 134 are receiving current derived from battery 194. According to some embodiments, the current to cartridge electric contacts 134 is monitored by processing unit 190, for achieving the evaporation heater 120 required temperature, as detailed herein. According to some embodiments, the current to cartridge electric contacts 134 drives from battery 194 through cartridge power coupling 196 and actuator power coupling 198.

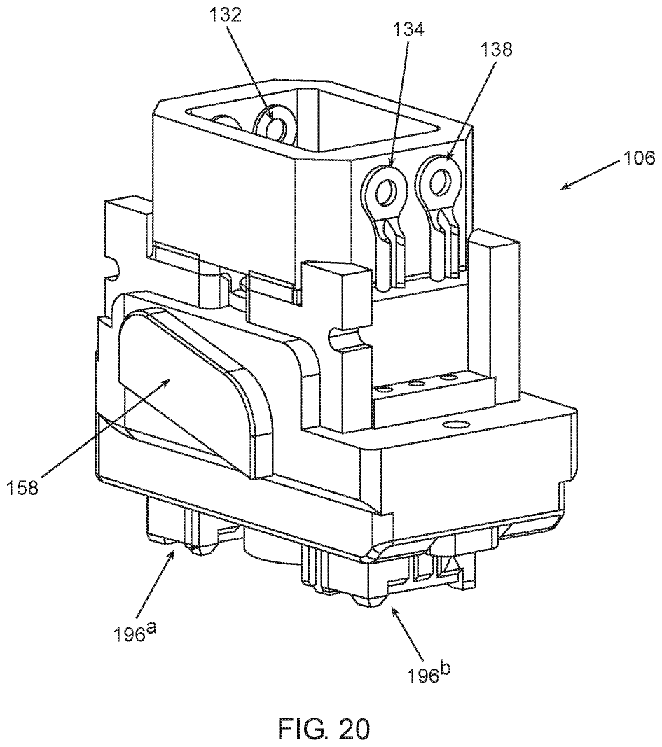

According to some embodiments, evaporation heater 120 may further comprise heater resistivity measurement contacts 136. According to some embodiments, heater resistivity measurement contacts 136 pass through support 122 to achieve an electric contact with matching output resistivity measurement contacts 138 (see e.g. FIG. 20). According to some embodiments, heater resistivity measurement contacts 136 and output resistivity measurement contacts 138 are configured to send resistivity signal to processing unit 190. According to some embodiments, the resistivity signal is indicative of the temperature of evaporation heater 120. According to some embodiments, processing unit 190 is configured to operate evaporation heater 120 in response to the resistivity signal. According to some embodiments, the operation signal comprises the resistivity signal.

Specifically, upon operation of electronic cigarette 100 processing unit 190 receives a first trigger activation signal and activates liquid deposition mechanism 160 and evaporation heater 120, according to some embodiments. According to some embodiments, evaporation heater 120 is heated upon its operation and receives a discrete volume of liquid from liquid deposition mechanism 160. According to some embodiments, the discrete volume of liquid is an aqueous formulation and when in contact with evaporation heater 120, it restricts the temperature of evaporation heater 120 not above the boiling temperature of water (100° C.) until the water in the discrete volume of liquid is evaporated. According to some embodiments, upon evaporation of the water in the discrete volume of liquid, the temperature of evaporation heater 120 rises and then higher boiling constituents are evaporated (e.g. nicotine or cannabis constituents). When the temperature of evaporation heater 120 rises, heater resistivity measurement contacts 136 send a resistivity signal indicative of the temperature rise through output resistivity measurement contacts 138, to processing unit 190, according to some embodiments. According to some embodiments, when processing unit 190 senses the temperature rising above a threshold temperature, it acts to prevent the temperature from rising. According to some embodiments, the threshold temperature is in the range of 300-400° C. According to some embodiments, processing unit 190 is configured to stop or reduce current drive to cartridge electric contacts 134 upon reaching or approaching the threshold temperature. According to some embodiments, processing unit 190 is configured to operate liquid deposition mechanism 160 to provide an additional discrete volume of liquid upon the resistivity signal indicating reaching or approaching the threshold temperature.

As detailed above, preferably a discrete and relatively thin layer of liquid needs to be dispersed over evaporation heater 120. A thin layer of liquid may be generally required for maintain evaporation heater 120 above a lower temperature limit, as detailed herein with respect to liquid deposition mechanism 160. Evaporation heater 120 evaporates the thin layer of liquid and dries quickly, since the thin layer contains small amount of material. A possible problem stemming from the quick drying is the overheating of evaporation heater 120, when it is not soaked in a liquid, which cools it. Specifically, evaporation heater 120 generates heat, wherein the heat is absorbed by the liquid in contact with evaporation heater 120. Accordingly, the liquid is evaporated. Upon evaporating, evaporation heater 120 is dried from the liquid and the formed heat is accumulated and its temperatures begins to rise. Moreover, when dealing with aqueous compositions, a relatively strong heater is required as evaporation heater 120 in order to overcome the substantial latent heat and specific heat capacity of water. Such overheating is avoided according to some embodiments, by the regulation of processing unit 190, which operates evaporation heater 120 in a manner that maintains its temperature below 300-450° C. For example, processing unit 190 may activate/deactivate evaporation heater 120 alternately to maintain this temperature, according to some embodiments. Processing unit 190 may apply differential current to evaporation heater 120 to maintain the required temperature range according to some embodiments.

As detailed herein the operation of aerosolizing the discrete thin layer of liquid may be performed by an evaporation heater, e.g. evaporation heater 120, which is responsible for both the action of generating heat and the action of providing a surface for the evaporation of the discrete thin layer of liquid, according to some embodiments. However, it is contemplated that two separate elements are included in electronic cigarette 100—a heater, responsible for generating heat and an evaporation medium, providing a surface for the evaporation of the discrete thin layer of liquid, according to some embodiments.

According to some embodiments, there is provided an e-cigarette, comprising an evaporation medium configured to receive a portion of a liquid for evaporation within or there upon. Preferably, the liquid comprises an aqueous nicotine formulation or an aqueous cannabinoid formulation. The e-cigarette further comprises and at least one heating element configured to heat the evaporation medium to a temperature high enough to facilitate creation and rupture of bubbles within the portion of the liquid for evaporation. It is to be understood that embodiments relating to elements of electronic cigarette 100 other than the evaporation medium and the at least one heater (e.g. liquid deposition assemblies, processing units, electric connections, triggers, outlets, actuators, etc.) may be presented in connection with an electronic cigarette having an evaporation heater, but similarly apply for an electronic cigarette having a separate heating element(s) and evaporation medium.

According to some embodiments, there is provided an electronic cigarette comprising a cartridge having a first end and a second end, the cartridge comprising an evaporation medium configured to evaporate a liquid from a surface thereof, at least one heating element configured to generate heat and to transfer the heat to the evaporation medium, a liquid drawing element; a liquid container; and an outlet; and an actuator having a first end and a second end, the actuator comprising a processing unit, wherein the first end of the actuator is connectable with the second end of the cartridge, wherein the electronic cigarette further comprises a first trigger configured to generate a first trigger activation signal, and a liquid deposition mechanism comprising the liquid drawing element and the liquid container, wherein the liquid drawing element is spaced apart from the evaporation medium in at least a first state of the electronic cigarette, and wherein the liquid deposition mechanism is configured to transfer a discrete volume of an aqueous formulation from the liquid drawing element to the evaporation medium in a second state of the electronic cigarette, wherein the liquid drawing element is in contact with the liquid container in both the first state of the electronic cigarette and the second state of the electronic cigarette, wherein the processing unit is configured to receive at least one operation signal and to control operations of at least one of the at least one heating element and the liquid deposition mechanism upon receiving the at least one operation signal, wherein the at least one operation signal comprises the first trigger activation signal.

According to some embodiments, there is provided electronic cigarette 100 comprising a cartridge 106 having a first end and a second end, the cartridge comprising an evaporation medium 320 configured to evaporate a liquid from a surface thereof, at least one heating element 330 configured to generate heat and to transfer the heat to heating element 330, a liquid drawing element 164; a liquid container 162; and an outlet 110; and an actuator 114 having a first end and a second end, the actuator comprising a processing unit 190, wherein the first end of actuator 114 is connectable with the second end of cartridge 106, wherein electronic cigarette 100 further comprises a first trigger 140 configured to generate a first trigger activation signal, and a liquid deposition mechanism 160 comprising liquid drawing element 164 and liquid container 162, wherein liquid drawing element 164 is spaced apart from evaporation medium 320 in at least a first state of the electronic cigarette, and wherein liquid deposition mechanism 160 is configured to transfer a discrete volume of an aqueous formulation from liquid drawing element 164 to the evaporation medium 320 in a second state of electronic cigarette 100, wherein liquid drawing element 164 is in contact with the liquid container 162 in both the first state of electronic cigarette 100 and the second state of electronic cigarette 100, wherein processing unit 190 is configured to receive at least one operation signal and to control operations of at least one of heating element 330 and the liquid deposition mechanism 160 upon receiving the at least one operation signal, wherein the at least one operation signal comprises the first trigger activation signal.

It is to be understood that embodiments referring to heating element 331 and evaporation medium 320 apply to any electronic cigarettes 100 as presented herein. Specifically, embodiments referring to evaporation heater 120 and FIGS. 7-11 apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

Specific embodiments relating to heating element 330 and evaporation medium 320 are shown in FIGS. 3C-D, 25, 26, 27A-B, 31A-B and 32A, which are discussed herein below.

According to some embodiments, electronic cigarette 100 further comprises a support 122. According to some embodiments, support 122 is rigidly attached to cartridge housing 102. According to some embodiments, evaporation medium 320 is attached to support 122 such that unintentional displacement of evaporation medium 320 upwards or downward in the longitudinal direction is prevented. According to some embodiments, evaporation medium 320 is attached to support 122 such that displacement of evaporation medium 320 upwards or downward in the longitudinal direction is prevented. According to some embodiments, evaporation medium 320 is attached to support 122 such that unintentional displacement of evaporation medium 320 in a non-longitudinal direction is prevented. According to some embodiments, evaporation medium 320 is attached to support 122 such that displacement of evaporation medium 320 in a non-longitudinal direction is prevented.

According to some embodiments, support 122 comprises a high-temperature resistant with low thermal conductivity material, such as conventional ceramics or aluminum silicate ceramics, titanium oxide, zirconium oxide, yttrium oxide ceramics, molten silicon, silicon dioxide and molten aluminum oxide. According to some embodiments, support 122 is made of ceramics.

According to some embodiments, processing unit 190 is configured to control the operation of heating element 330 and thereby to regulate the temperature of evaporation medium 320.

According to some embodiments, heating element 330 is housed within cartridge housing 102. According to some embodiments, evaporation medium 320 is housed within cartridge housing 102.

Without wishing to be bound by any theory or mechanism of action, upon heating of evaporation medium 320 by heating element 330, the liquid is at least partially vaporized into vapor. Subsequently, the vapor in condensed into aerosol, which may be inhaled by a user in need thereof, such as an electronic cigarette user.

According to some embodiments, evaporation medium 320 is rigid. According to some embodiments, evaporation medium 320 is made of metal. According to some embodiments, evaporation medium 320 has two flat sides, which remain flat when liquid is pressed there through. According to some embodiments, evaporation medium 320 has a top flat surface and a bottom flat surface, which do not deform when liquid is pressed there through or pressed against at least one of the top surface or the bottom surface.

According to some embodiments, heating element 330 is configured to provide 3-7 W, 4-6 W, 4.5-5.9 W, 4.8-5.6 W, 5.0-5.4 W or 5.1-5.3 W per every μl of liquid deposited thereon.

According to some embodiments, heating element 330 is configured to provide about 5.2 W per every μl of liquid deposited thereon.

According to some embodiments evaporation medium 320 has heat capacity of no more than 1000 Jkg⁻¹K⁻¹. According to some embodiments evaporation medium 320 has heat capacity of no more than 900 Jkg⁻¹K⁻¹. According to some embodiments, evaporation medium 320 has heat capacity of no more than 800 Jkg⁻¹K⁻¹. According to some embodiments, evaporation medium 320 has heat capacity of no more than 700 Jkg⁻¹K⁻¹. According to some embodiments, evaporation medium 320 has heat capacity of no more than 600 Jkg⁻¹K⁻¹.

According to some embodiments, evaporation medium 320 has a specific heat capacity in the range of 100 to 900 Jkg⁻¹K⁻¹. According to some embodiments, evaporation medium 320 has a specific heat capacity in the range of 200 to 800 Jkg⁻¹K⁻¹. According to some embodiments, evaporation medium 320 has a specific heat capacity in the range of 300 to 750 Jkg⁻¹K⁻¹. According to some embodiments, evaporation medium 320 has a specific heat capacity in the range of 400 to 700 Jkg⁻¹K⁻¹. According to some embodiments, evaporation medium 320 has a specific heat capacity in the range of 450 to 650 Jkg⁻¹K⁻¹. According to some embodiments, evaporation medium 320 has a specific heat capacity in the range of 500 to 600 Jkg⁻¹K⁻¹. According to some embodiments, evaporation medium 320 has a specific heat capacity in the range of 470 to 570 Jkg⁻¹K⁻¹. According to some embodiments evaporation medium 320 has a specific heat capacity in the range of 485 to 555 Jkg⁻¹K⁻¹. According to some embodiments, evaporation medium 320 has a specific heat capacity in the range of 500 to 540 Jkg⁻¹K⁻¹.

According to some embodiments, evaporation medium 320 has surface heat flux in the range of 170 Wcm⁻² to 290 Wcm⁻². According to some embodiments, evaporation medium 320 has surface heat flux in the range of 200 Wcm⁻² to 260 Wcm⁻². According to some embodiments, evaporation medium 320 has surface heat flux in the range of 210 Wcm⁻² to 250 Wcm⁻². According to some embodiments, evaporation medium 320 has surface heat flux in the range of 220 Wcm⁻² to 240 Wcm⁻². According to some embodiments, evaporation medium 320 has surface heat flux of about 228 Wcm⁻².

According to some embodiments, heating element 330 is configured to provide an energy output of at least 20 Joules within half a second. According to some embodiments, heating element 330 is configured to provide an energy output of at least 25 Joules within half a second. According to some embodiments, heating element 330 is configured to provide an energy output of at least 30 Joules within half a second. According to some embodiments, heating element 330 is configured to provide an energy output of at least 35 Joules within half a second. According to some embodiments, heating element 330 is configured to provide an energy output of at least 40 Joules within half a second. According to some embodiments, heating element 330 is configured to provide an energy output of at least 45 Joules within half a second. According to some embodiments, heating element 330 is configured to provide an energy output of at least 50 Joules within half a second. According to some embodiments, heating element 330 is configured to provide an energy output of at least 51 Joules within half a second.

According to some embodiments, evaporation medium 320 has a total resistance in the range of 0.10Ω to 0.60Ω. According to some embodiments, evaporation medium 320 has a total resistance in the range of 0.13Ω to 0.55Ω. According to some embodiments, evaporation medium 320 has a total resistance in the range of 0.15Ω to 0.5Ω. According to some embodiments, evaporation medium 320 has a total resistance in the range of 0.15Ω to 0.45Ω. According to some embodiments, evaporation medium 320 has a total resistance in the range of 0.2Ω to 0.4Ω.

According to some embodiments, heating element 330 is configured to provide an energy output of at least 50 Watts. According to some embodiments, heating element 330 is configured to provide an energy output of at least 60 Watts According to some embodiments, heating element 330 is configured to provide an energy output of at least 70 Watts According to some embodiments, heating element 330 is configured to provide an energy output of at least 80 Watts. According to some embodiments, heating element 330 is configured to provide an energy output of at least 90 Watts. According to some embodiments, heating element 330 is configured to provide an energy output of at least 100 Watts. According to some embodiments, heating element 330 is configured to provide an energy output of at least 102 Watts.

According to some embodiments, evaporation medium 320 is configured to drive current in the range of 10 A and 40 A. According to some embodiments, evaporation medium 320 is configured to drive current in the range of 15 A and 35 A. According to some embodiments, evaporation medium 320 is configured to drive current in the range of 20 A and 30 A. According to some embodiments, evaporation medium 320 is configured to drive current in the range of 25 A and 30 A. According to some embodiments, evaporation medium 320 is configured to drive current of about 28 A.

According to some embodiments, evaporation medium 320 is disposable. According to some embodiments, evaporation medium 320 is in the form of a rod, a capsule or a flat disc.

According to some embodiments, evaporation medium 320 comprises a thermally-conductive material, such as metal. According to some embodiments, heating element 330 comprises a thermally-conductive material, such as metal.

According to some embodiments, evaporation medium 320 has thermal mass of not more than 0.3 J/C. According to some embodiments, evaporation medium 320 has thermal mass of not more than 0.2 J/C. According to some embodiments evaporation medium 320 has thermal mass of not more than 0.1 J/C. According to some embodiments, evaporation medium 320 has thermal mass of less than 0.1 J/C. According to some embodiments, evaporation medium 320 has thermal mass of less than 0.08 J/C. According to some embodiments, evaporation medium 320 has thermal mass of less than 0.06 J/C. According to some embodiments, evaporation medium 320 has thermal mass of less than 0.04 J/C. According to some embodiments, evaporation medium 320 has thermal mass of less than 0.3 J/C. According to some embodiments, evaporation medium 320 has thermal mass of less than 0.2 J/C.

According to some embodiments, evaporation medium 320 has thermal mass in the range of 0.001 J/C to 0.3 J/C. According to some embodiments, evaporation medium 320 has thermal mass in the range of 0.004 J/C to 0.25 J/C. According to some embodiments, evaporation medium 320 has thermal mass in the range of 0.006 J/C to 0.2 J/C. According to some embodiments, evaporation medium 320 has thermal mass in the range of 0.01 J/C to 0.015 J/C.

According to some embodiments, evaporation medium 320 made of a uniform material. According to some embodiments, evaporation medium 320 is made of metal. According to some embodiments, evaporation medium 320 comprises a metal and/or a metal alloy. According to some embodiments, evaporation medium 320 comprises a metal alloy. According to some embodiments, evaporation medium 320 comprises at least one metal selected from iron, nickel, titanium, chromium, aluminum, molybdenum and manganese. According to some embodiments, the alloy comprises at least one metal selected from iron, nickel, titanium, chromium, aluminum, molybdenum, silver, palladium and manganese. Each possibility represents a separate embodiment. According to some embodiments evaporation medium 320 comprises a metal having electrical resistivity in the range of 0.3·10⁻⁶ to 3·10⁻⁶Ω·m at room temperature. According to some embodiments, evaporation medium 320 comprises a metal having electrical resistivity in the range of 0.4·10⁻⁶ to 2.5·10⁻⁶ Ω·m at room temperature. According to some embodiments, evaporation medium 320 comprises a metal having electrical resistivity in the range of 0.5·10⁻⁶ to 2·10⁻⁶ Ω·m at room temperature. According to some embodiments, evaporation medium 320 comprises a metal having electrical resistivity in the range of 0.6·10⁻⁶ to 1.5·10⁻⁶ Ω·m at room temperature. According to some embodiments, evaporation medium 320 comprises an alloy having electrical resistivity in the range of 0.3·10⁻⁶ to 3·10⁻⁶ Ω·m at room temperature. According to some embodiments, evaporation medium 320 comprises an alloy having electrical resistivity in the range of 0.4·10⁻⁶ to 2.5·10⁻⁶ Ω·m at room temperature. According to some embodiments, evaporation medium 320 comprises an alloy having electrical resistivity in the range of 0.5·10⁻⁶ to 2·10⁻⁶ Ω·m at room temperature. According to some embodiments, evaporation medium 320 comprises an alloy having electrical resistivity in the range of 0.6·10⁻⁶ to 1.5·10⁻⁶ Ω·m at room temperature. According to some embodiments, the alloy is selected from Kanthal, Nichrome and stainless steel. According to some embodiments, the alloy is Nichrome. According to some embodiments, the alloy is stainless steel. According to some embodiments, the alloy is 316L stainless steel.

According to some embodiments, heating element 330 is configured to generate heat rapidly, such that its temperature elevates rapidly.

According to some embodiments, heating element 330 is configured to generate sufficient heat so as to elevate its temperature to a value high enough to at least partially evaporate the liquid contained by or in direct contact with heating element 330, thereby enabling electronic cigarette 100 to produce vapor comprising a constant and reproducible dose. According to some embodiments, heating element 330 is configured to generate sufficient heat so as to elevate its temperature to a value high enough to at least partially evaporate water contained by or in direct contact with heating element 330, thereby enabling electronic cigarette 100 to produce water vapor comprising a constant and reproducible dose. According to some embodiments, heating element 330 is configured to generate sufficient heat so as to elevate its temperature to a value high enough to at least partially vaporize nicotine contained by or in direct contact with heating element 330, thereby enabling electronic cigarette 100 to produce nicotine vapor comprising a constant and reproducible dose. According to some embodiments, heating element 330 is configured to generate sufficient heat so as to elevate its temperature to a value high enough to at least partially vaporize cannabinoid(s) contained by or in direct contact with heating element 330, thereby enabling electronic cigarette 100 to produce cannabinoid vapor comprising a constant and reproducible dose. According to some embodiments, heating element 330 is configured to generate sufficient heat so as to elevate its temperature to a value high enough to substantially evaporate the liquid contained by or in direct contact with heating element 330, thereby enabling electronic cigarette 100 to produce vapor comprising a constant and reproducible dose. According to some embodiments, heating element 330 is configured to generate sufficient heat so as to elevate its temperature to a value high enough to substantially evaporate water contained by or in direct contact with heating element 330, thereby enabling electronic cigarette 100 to produce water vapor comprising a constant and reproducible dose. According to some embodiments, heating element 330 is configured to generate sufficient heat to so as to elevate its temperature to a value high enough to substantially evaporate nicotine contained by or in direct contact with heating element 330, thereby enabling electronic cigarette 100 to produce nicotine vapor comprising a constant and reproducible dose. According to some embodiments, heating element 330 is configured to generate sufficient heat to so as to elevate its temperature to a value high enough to substantially evaporate cannabinoid(s) contained by or in direct contact with heating element 330, thereby enabling electronic cigarette 100 to produce cannabinoid vapor comprising a constant and reproducible dose.

According to some embodiments, heating element 330 is configured to generate heat, to reach a temperature in the range between 50 and 600 degrees Celsius. According to some embodiments, the temperature is at least 95° C., at least 96° C., at least 97° C., at least 98° C., at least 98.5° C., at least 99° C., at least 99.5° C., or at least 100° C. According to some embodiments, the temperature is not more than 600° C., not more than 550° C., not more than 500° C., not more than 450° C., not more than 400° C., not more than 350° C. or not more than 300° C.

According to some embodiments, heating element 330 comprises a resistive heater. According to some embodiments, heating element 330 comprises a radio-frequency heater. According to some embodiments, heating element 330 comprises an induction-coil heater.

According to some embodiments, heating element 330 is in direct contact with evaporation medium 320, to conduct heat thereto.

Without wishing to be bound by any theory or mechanism of action, heating element 330 is required to include a relatively strong heater. Specifically, electronic cigarette 100 is designed to evaporate aqueous compositions, according to some embodiments. Thus use of water, however, may pose several obstacles. Importantly, water has a high latent heat value, meaning that substantial energy has to be invested in order to evaporate water. Thus, according to some embodiments, heating element 330 is a strong heater configured to generate enough heat to vaporize an aqueous solution of nicotine (2-5%) at a rate of at least 0.01 mg nicotine per second, which is considered to provide satisfying consumer experience. Also, according to some embodiments, heating element 330 is a strong heater configured to generate enough heat to vaporize an aqueous solution of cannabinoid (1-10%) at a rate of at least 0.025 mg THC per second, which is considered to provide satisfying consumer experience. According to some embodiments, heating element 330 is configured to generate at least 20 W power. According to some embodiments, heating element 330 is configured to generate at least 32 W power. According to some embodiments, heating element 330 is configured to generate at least 34 W power. According to some embodiments, heating element 330 is configured to generate at least 36 W power. According to some embodiments, heating element 330 is configured to generate at least 8 W power. According to some embodiments, heating element 330 is configured to generate at least 40 W power.

An additional obstacle encountered when dealing with evaporation aqueous composition, is the slow evaporation thereof, which stems from the high specific heat capacity of water as well as from the high latent heat of water. Both these high values entail investment of a substantial amount of energy, which in turn, is slower than when using organic formulations (i.e. PG or VG). Thus, together with high electrical power, an additional requirement from evaporation medium 320 is directed to it low thermal mass. According to some embodiments, evaporation medium 320 has thermal mass of less than 0.05 J/C.

According to some embodiments, heating element 330 is a strong heater configured to generate enough heat to evaporation medium 320 so as to vaporize an aqueous solution of nicotine (2-5%) at a rate of at least 0.1 mg nicotine per second, which is considered to provide satisfying consumer experience.

According to some embodiments, heating element 330 is a strong heater configured to generate enough heat to evaporation medium 320 so as to vaporize an aqueous solution of nicotine (2-5%) at a rate of at least 0.25 mg THC per second, which is considered to provide satisfying consumer experience. It is to be understood that when using the cannabinoid

According to some embodiments, processing unit 190 is configured to receive at least one operation signal and to control operation of heating element 330 upon receiving the at least one operation signal. According to some embodiments, processing unit 190 is configured to regulate the temperature of evaporation medium 320. According to some embodiments, the regulation entails maintaining the temperature of evaporation medium 320 in the range of 95° C. to 400° C. Preferably, the temperature of evaporation medium 320 is maintained in the range of 99.5° C. to 350° C. According to some embodiments, processing unit 190 is configured to control operation of heating element 330, which generates heat and elevates the temperature of evaporation medium 320, thereby regulating the temperature of evaporation medium 320.

Evaporation medium 320 may take the shape of evaporation heater 120, as shown in FIGS. 7 and 9 and described above, wherein evaporation medium 320 is in contact with heating element 330. In such case evaporation heater electric contact 132 is replaced with heating element 330, which is connected to a similar electrical contact.

It was surprisingly found that the formation of evaporation medium 320 with inner tracks 124 results in the reduction or elimination of the Leidenfrost effect, which is associated with aqueous formulations, according to some embodiments. Thus, an additional beneficial feature of inner tracks 124 is that it enables the prevention of the Leidenfrost effect. Specifically, inner tracks 124 act as capillary channel, which reduce Leidenfrost effect using a capillary effect, which is not enabled by standard flat impervious heaters or evaporation media connected to heaters.

It is an additional beneficial feature of inner tracks 124 that the enable the discrete volume of liquid deposited on evaporation medium 320 to cling on to the bottom surface of evaporation medium 320. Specifically, upon deposition of the discrete volume of liquid on a flat uniform/impermeable heated surface, portions of the discrete volume of liquid may ‘bounce’ off the bottom surface downwards, whereas the capillary structure of elongated heat conductive coil 126 and inner tracks 124 prevents or reduces the bounce-off effect. The capillary tracks are also very important in “pinning the liquid” to the evaporation surface especially when using the piezo option. Without these tracks, liquid ejected by the piezo would bounce off the heater.

Specifically, upon operation of electronic cigarette 100 processing unit 190 receives a first trigger activation signal and activates liquid deposition mechanism 160 and heating element 330, according to some embodiments. According to some embodiments, evaporation medium 320 is heated upon the operation of heating element 330 and receives a discrete volume of liquid from liquid deposition mechanism 160. According to some embodiments, the discrete volume of liquid is an aqueous formulation and when in contact with evaporation medium 320, it restricts the temperature of evaporation medium 320 not above the boiling temperature of water (100° C.) until the water in the discrete volume of liquid is evaporated. According to some embodiments, upon evaporation of the water in the discrete volume of liquid, the temperature of evaporation medium 320 rises and then higher boiling constituents are evaporated (e.g. nicotine or cannabis constituents). When the temperature of evaporation medium 320 rises, heater resistivity measurement contacts 136 sends a resistivity signal indicative of the temperature rise through output resistivity measurement contacts 138, to processing unit 190, according to some embodiments. According to some embodiments, when processing unit 190 senses the temperature rising above a threshold temperature, it acts to prevent the temperature from rising. According to some embodiments, the threshold temperature is in the range of 300-400° C. According to some embodiments, processing unit 190 is configured to stop or reduce current drive to cartridge electric contacts 134 upon reaching or approaching the threshold temperature. According to some embodiments, processing unit 190 is configured to operate liquid deposition mechanism 160 to provide an additional discrete volume of liquid upon the resistivity signal indicating reaching or approaching the threshold temperature.

As detailed above, preferably a discrete and relatively thin layer of liquid needs to be dispersed over evaporation medium 320. A thin layer of liquid may be generally required for maintain evaporation medium 320 above a lower temperature limit, as detailed herein with respect to liquid deposition mechanism 160. Evaporation medium 320 evaporates the thin layer of liquid and dries quickly, since the thin layer contains small amount of material. A possible problem stemming from the quick drying is the overheating of evaporation medium 320, when it is not soaked in a liquid, which cools it. Specifically, heating element 330 generates heat, wherein the heat is absorbed by evaporation medium 320 and therefore in the liquid in contact therewith. Accordingly, the liquid is evaporated. Upon evaporating, evaporation medium 320 is dried from the liquid and the formed heat is accumulated and its temperatures begins to rise. Moreover, when dealing with aqueous compositions, a relatively strong heater is required as heating element 330 in order to overcome the substantial latent heat and specific heat capacity of water. Such overheating is avoided according to some embodiments, by the regulation of processing unit 190, which operates heating element 330 in a manner that maintains its temperature below 300-450° C. For example, processing unit 190 may activate/deactivate heating element 330 alternately to maintain this temperature, according to some embodiments. Processing unit 190 may apply differential current to heating element 330 to maintain the required temperature range according to some embodiments.

With specific reference to FIGS. 3C-D it is noted that electronic cigarette 100 as presented in FIGS. 3C-D is similar to electronic cigarette 100 as presented in FIGS. 3A-B and its operation is similar with respect to the phases of electronic cigarette 100 and the liquid deposition. The difference between electronic cigarette 100 as presented in FIGS. 3C-D and electronic cigarette 100 as presented in FIGS. 3A-B, is that electronic cigarette 100 of FIGS. 3A-B comprises evaporation heater 120, whereas electronic cigarette 100 of FIGS. 3C-D comprises evaporation medium 320 and two heating elements 330^{a} and 330^{b} instead.

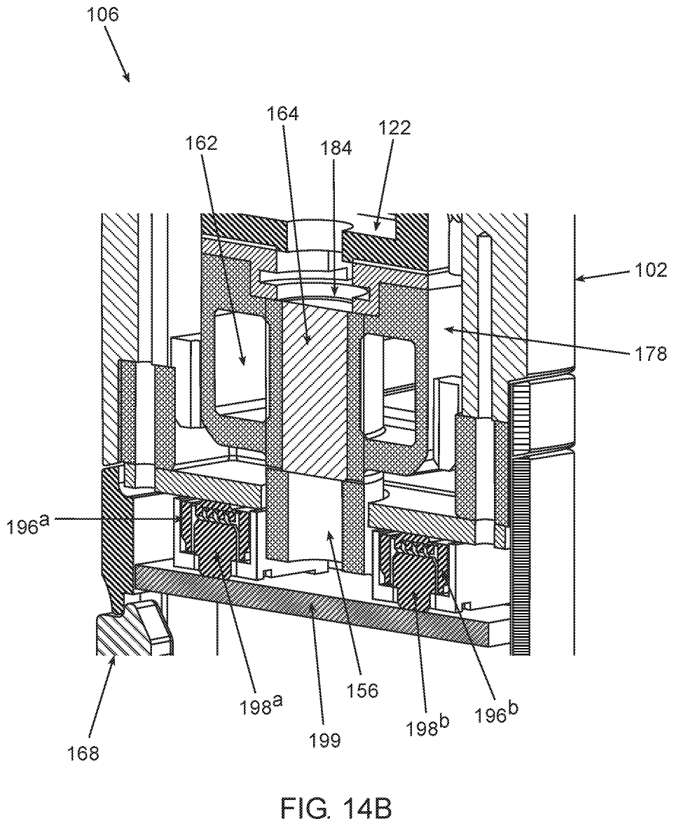

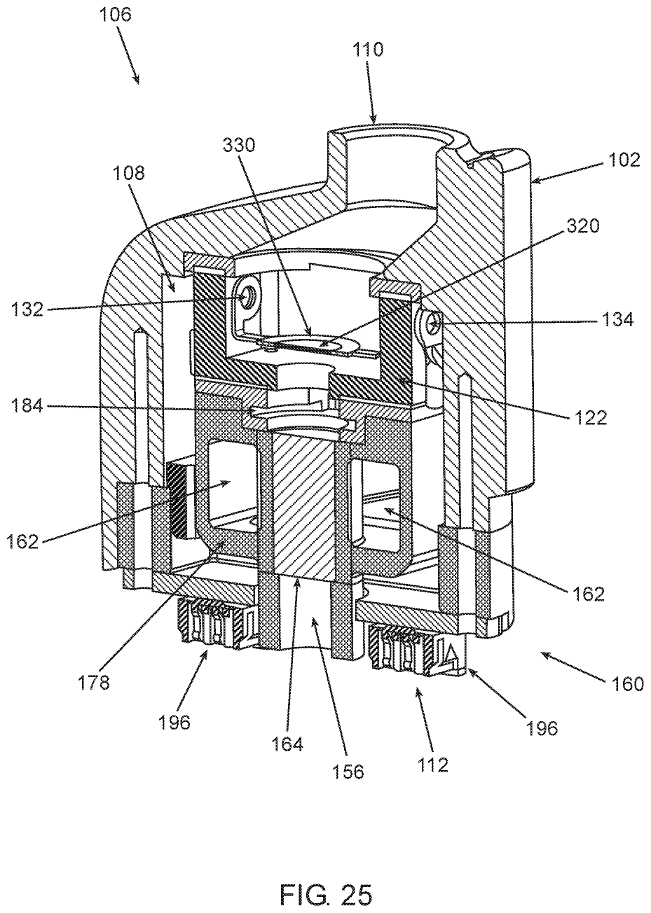

With specific reference to FIGS. 25, 26, 27A-B it is noted that cartridge 106 as presented in FIGS. 25, 26, 27A-B is similar to cartridge 106 as presented in FIGS. 14A, 17 and 18A-B. Cartridge 106 as presented in FIGS. 25, 26, 27A-B may be used as part of electronic cigarette 100 as presented in FIGS. a and 5, and its operation is similar with respect to the phases of electronic cigarette 100 and the liquid deposition. The difference between cartridge 106 as presented in FIGS. 25, 26, 27A-B and cartridge 106 as presented in FIGS. 14A, 17 and 18A-B, is that cartridge 106 of FIGS. 25, 26, 27A-B comprises evaporation heater 120, whereas cartridge 106 of FIGS. 14A, 17 and 18A-B comprises evaporation medium 320 and heating element 330 instead. Heating element 330 of FIGS. 25, 26, 27A-B surrounds evaporation medium 320 and is in contact with its circumference.

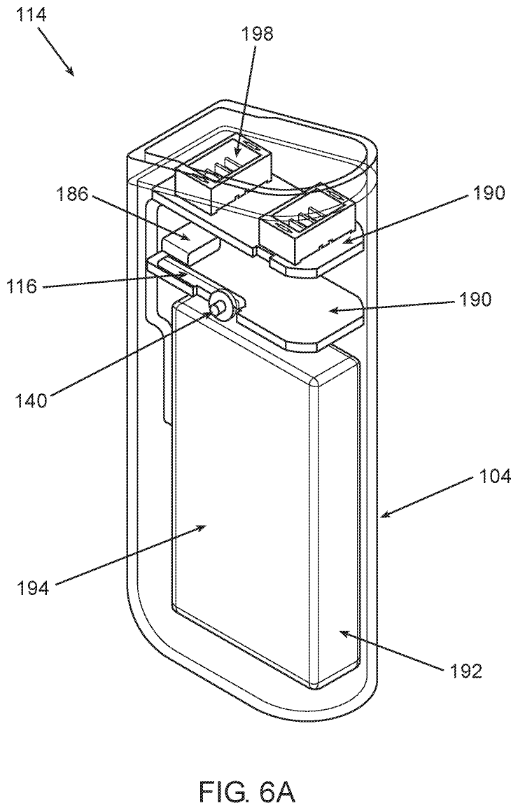

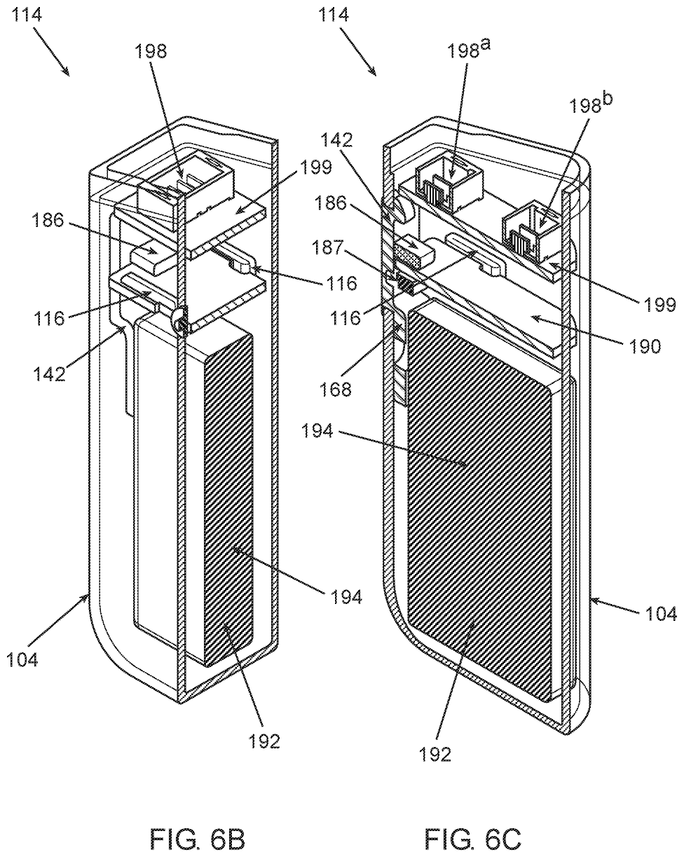

Reference is made back to different elements of electronic cigarette 100, as shown in the figures. According to some embodiments, electronic cigarette 100 comprises a first trigger 140, configured to at least trigger activation or deactivation of at least one of evaporation heater 120 and liquid deposition mechanism 160. According to some embodiments, first trigger 140 is a switch. According to some embodiments, first trigger 140 is a knob. According to some embodiments, first trigger 140 is a dial. According to some embodiments, first trigger 140 is a lever. According to some embodiments, first trigger 140 is a button. According to some embodiments, first trigger 140 is a touch interface. According to some embodiments, first trigger 140 is a force sensor. According to some embodiments, first trigger 140 is a pressure sensor. According to some embodiments, first trigger 140 is a flow sensor. First trigger 140 is portrayed in FIG. 4 and FIG. 6A as a button, which is optional.

It is to be understood that embodiment referring to first trigger 140 and processing unit 190 apply to any electronic cigarettes 100 as presented herein. Specifically, embodiments referring to first trigger 140 and processing unit 190 apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

According to some embodiments, first trigger 140 is a proximity sensor. According to some embodiments, the proximity sensor is attached to an exterior surface of cartridge housing 102. According to some embodiments, the proximity sensor is positioned in the vicinity of outlet 110. According to some embodiments, the proximity sensor is configured to detect proximity to a user's mouth.

According to some embodiments, processing unit 190 is configured to receive signals from first trigger 140. According to some embodiments, first trigger 140 is configured to generate at least a first trigger activation signal. According to some embodiments, evaporation heater 120 is configured to generate heat when first trigger 140 generates the first trigger activation signal.

According to some embodiments, evaporation heater 120 is configured to generate heat when first trigger 140 generates the first trigger activation signal. According to some embodiments, processing unit 190 is configured to operate evaporation heater 120, such that it generates heat, upon receipt of first trigger activation signal, when generated by first trigger 140.

According to some embodiments, processing unit 190 is configured to control operation evaporation heater 120. According to some embodiments, processing unit 190 is configured to activate evaporation heater 120 upon receiving first trigger activation signal from first trigger 140. According to some embodiments, processing unit 190 is configured to deactivate evaporation heater 120 upon stopping receiving first trigger activation signal from first trigger 140.

According to some embodiments, first trigger 140 is further configured to generate a deactivation signal. According to some embodiments, the at least one operation signal comprises the deactivation signal. According to some embodiments, processing unit 190 is configured to deactivate evaporation heater 120 upon receiving first trigger deactivation signal from first trigger 140.

According to some embodiments, processing unit 190 is configured to activate both evaporation heater 120 and liquid deposition mechanism 160 upon receiving first trigger activation signal. According to some embodiments, processing unit 190 is configured to deactivate both evaporation heater 120 and liquid deposition mechanism 160 upon stopping receiving first trigger activation signal from first trigger 140.

According to some embodiments, first trigger 140 is configured to generate a variable first trigger activation signal, varying in at least one of: amplitude, wavelength or frequency of the signals. According to some embodiments, processing unit 190 is configured to provide varying activation signals to liquid deposition mechanism 160, thereby controlling various parameters of liquid deposition mechanism 160 as a function of the first trigger activation signals generated by first trigger 140.

For example, first trigger 140 may be a touch user interface, according to some embodiments. According to some embodiments, the user interface may provide options to a user for determining parameters by which processing unit 190 controls liquid deposition mechanism 160 and/or evaporation heater 120. According to some embodiments, the touch user interface is configured to provide to an electronic cigarette 100 user at least two sensorial options. According to some embodiments, upon selecting each of the at least two sensorial options, at least one control parameter of processing unit 190 over liquid deposition mechanism 160 are executed. According to some embodiments, the at least one control parameter is selected from fluid deposition frequency and fluid deposition duty cycle.

According to some embodiments, the fluid deposition frequency is in the range of 0.5 Hz to 50 Hz. According to some embodiments, the fluid deposition frequency is in the range of 0.75 Hz to 40 Hz. According to some embodiments, the fluid deposition frequency is in the range of 1 Hz to 30 Hz. According to some embodiments, the fluid deposition frequency is in the range of 1.5 Hz to 25 Hz. According to some embodiments, the fluid deposition frequency is in the range of 2 Hz to 20 Hz. According to some embodiments, the fluid deposition frequency is in the range of 2 Hz to 10 Hz.

According to some embodiments, the duty cycle is in the range of 5% to 80%. According to some embodiments, the duty cycle is in the range of 7% to 70%. According to some embodiments, the duty cycle is in the range of 10% to 60%. According to some embodiments, the duty cycle is in the range of 12% to 50%. According to some embodiments, the duty cycle is in the range of 14% to 40%. According to some embodiments, the duty cycle is in the range of 15% to 35%. According to some embodiments, the duty cycle is in the range of 20% to 30%.

The phrase “fluid deposition frequency” refers to the number of times in which liquid deposition mechanism 160 deposits discrete volume of liquid onto evaporation heater 120 per time unit. Alternatively, the phrase “fluid deposition frequency” refers to the number of times in which electronic cigarette 100 transforms from the first state to the second state of action per time unit.

The phrase “fluid deposition frequency” refers to the time ratio between the first state and the second state of electronic cigarette 100. As detailed herein during the second state, a discrete volume of liquid is delivered to evaporation heater 120, and during the first state liquid is not delivered to evaporation heater 120. Thus, the phrase “fluid deposition frequency” refers to the relative duration in which evaporation heater 120 is being deposited with liquid.

According to some embodiments, upon selecting each of the at least two sensorial options, at least one control parameter of processing unit 190 over evaporation heater 120 are executed. According to some embodiments, the at least one control parameter comprises evaporation heater 120 threshold temperature. As detailed herein, the threshold temperature is the temperature above which, processing unit 190 stops driving current- or reducing the current driven to evaporation heater 120, for its heating.

According to some embodiments, first trigger 140 is further configured to generate a deactivation signal, such that processing unit 190 is configured to deactivate both solenoid actuator 170 and evaporation heater 120 upon receiving first trigger deactivation signal from first trigger 140.

Thus, according to some embodiments, processing unit 190 is configured to regulate the temperature of evaporation heater 120 in the range of 95° C. to 400° C., through control of the operation of both evaporation heater 120 and liquid deposition mechanism 160. According to some embodiments, processing unit 190 is configured to regulate the temperature of evaporation heater 120 below 400° C., below 350° C., or below 330° C., through control of the operation of liquid deposition mechanism 160 and/or evaporation heater 120. According to some embodiments, processing unit 190 is configured to receive at least one operation signal and to control operation of liquid deposition mechanism 160.

According to some embodiments, processing unit 190 is configured to regulate the temperature of evaporation heater 120 above the nicotine-water azeotropic temperature of 99.5° C. According to some embodiments, the regulation entails providing variable current to cartridge electric contacts 134 as detailed above. Specifically, it is to be understood that deposition of liquid over evaporation heater 120 effects its temperature.

According to some embodiments, electronic cigarette 100 further comprises a power source compartment 192 (FIGS. 1-5 and 6A-C), configured to house at least one power source, such as a battery 194. It is to be understood that embodiment referring to power source compartment 192 and battery 194 apply to any electronic cigarettes 100 as presented herein. Specifically, embodiments referring to power source compartment 192 and battery 194 apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

Battery 194 is configured to provide electric current to processing unit 190, evaporation heater 120, evaporation heater electric contact 132, cartridge electric contacts 134, flow or pressure sensor 152, liquid deposition mechanism 160 solenoid actuator 170, piezo disc 180, cartridge power coupling 196 and actuator power coupling 198, according to some embodiments. According to some embodiments, battery 194 is configured to provide electric current to processing unit 190 directly. According to some embodiments, battery 194 is configured to provide electric current to evaporation heater 120 through evaporation heater electric contact 132 and cartridge electric contacts 134. According to some embodiments, battery 194 is configured to provide electric current to flow or pressure sensor 152 directly. According to some embodiments, battery 194 is configured to provide electric current to solenoid actuator 170 through cartridge power coupling 196 and actuator power coupling 198. According to some embodiments, battery 194 is configured to provide electric current to piezo disc 180 through cartridge power coupling 196 and actuator power coupling 198. The current to evaporation heater electric contact 132 drives through cartridge power coupling 196 and actuator power coupling 198, according to some embodiments.

According to some embodiments, battery 194 may be a rechargeable or disposable battery. Specifically, battery 194 may be a relatively strong power source, since, as detailed herein, evaporation heater 120 is configured to generate high electrical wattage. According to some embodiments, the battery 194 is at least one Lipo battery. According to some embodiments, battery 194 has voltage of about 3.7V. According to some embodiments, battery 194 is a battery having voltage of about 3.7V. According to some embodiments, battery 194 has maximum discharge current of about 40 A. According to some embodiments, battery 194 has capacity of about 1400 mAh (milli-Ampere-hour). According to some embodiments, battery 194 has C-rating value of about 30. According to some embodiments, power source compartment 192 comprises battery 194.

Although evaporation heater 120 having the structure shown in FIGS. 7-11 is detailed herein, other forms of evaporation heater 120 are contemplated. The additional configurations of evaporation heater 120 have similar heat conducting, material and resistivity properties. Specific options for such evaporation heater include an evaporation heater having at least one surface with high roughness, and an evaporation heater having porous medium.

According to some embodiments, evaporation heater 120 comprises a heater having a bottom surface with high roughness, wherein the degree of roughness is configured to form a high liquid-contact area.

According to some embodiments, evaporation heater 120 comprises a heater having a distal surface with high roughness, wherein the degree of roughness is configured to form a high liquid-contact area.

According to some embodiments, evaporation heater 120 comprises a non-porous heater having a distal surface with high roughness, wherein the degree of roughness is configured to form a high liquid-contact area.

According to some embodiments, evaporation heater 120 comprises a distal surface with high roughness, wherein the degree of roughness forms the high liquid-contact area; or wherein evaporation heater 120 comprises a porous medium, wherein pores of the porous medium forms the high liquid-contact area.

According to some embodiments, evaporation heater 120 is produced through a process, which comprises a step of bead-blasting, thereby achieving surface roughness.

According to some embodiments, evaporation heater 120 comprises a porous medium, wherein pores of the porous medium are configured to form a high liquid-contact area.

According to some embodiments, evaporation heater 120 formed as a porous medium is rigid where liquid is absorbed, or partially absorbed, therein.

According to some embodiments, evaporation heater 120 has a proximal flat surface (not numbered) and a distal high-roughness surface, which do not deform when liquid is pressed there through or pressed against at least one of the proximal surface or the distal surface.

According to some embodiments, evaporation heater 120 has a projected surface area of 1-3 or 1.5-2.7 mm² per every μl of liquid deposited onto it.

According to some embodiments, evaporation heater 120 has a projected surface area of about 2.3 mm² per every μl of liquid deposited onto it.

According to some embodiments, evaporation heater 120 has a projected surface area in the range of 1 mm² to 100 mm². According to some embodiments, evaporation heater 120 has a projected surface area in the range of 5 mm² to 90 mm². According to some embodiments, evaporation heater 120 has a projected surface area in the range of 10 mm² to 80 mm². According to some embodiments, evaporation heater 120 has a projected surface area in the range of 20 mm² to 70 mm². According to some embodiments, evaporation heater 120 has a projected surface area in the range of 30 mm² to 60 mm². According to some embodiments, evaporation heater 120 has a projected surface area in the range of 40 mm² to 50 mm². According to some embodiments, the heater surface has a projected surface area of about 45 mm².

According to some embodiments, evaporation heater 120 comprises high liquid-contact area, configured to elevate the Leidenfrost temperature to avoid the Leidenfrost effect.

The term “high liquid-contact area” pertaining to an evaporation heater, as used herein, refers to a surface area for contacting liquid being at least one order of magnitude higher than the surface area of a flat non-porous medium having the same external dimensions. For example, a study published Geraldi et al. (“Leidenfrost transition temperature for stainless steel meshes”, Materials Letters, 2016) showed that increasing the open area of a metal mesh pushes up the Leidenfrost temperature from 265° C. for an open area of 0.004 mm² to 315° C. for open area of 0.100 mm².

According to some embodiments, evaporation heater 120 is rigid. According to some embodiments, evaporation heater 120 is made of metal. According to some embodiments, evaporation heater 120 has two flat sides, which remain flat when liquid is pressed there through. According to some embodiments, evaporation heater 120 formed as a porous medium is rigid where liquid is partially absorbed, therein. According to some embodiments, evaporation heater 120 has a proximal flat surface and a distal high-roughness surface, which do not deform when liquid is pressed there through or pressed against at least one of the proximal surface or the distal surface.

The term “partially absorbed” and “partially saturated”, as used herein, are interchangeable and refer to the percentage of liquid absorbed in the pores of the porous material, wherein 0% refers to a porous material where all of its pores are vacant of liquid. Thus, the term “partially absorbed” may refer to a porous material wherein at least 0.005% of the pores contain liquid, or wherein the overall contents of the vacant space within the porous material occupied with liquid is 0.005%. According to some embodiments, partially absorbed refers to at least 0.001% liquid contents within the porous material. According to some embodiments, partially absorbed refers to at least 0.05% liquid contents within the porous material. According to some embodiments, partially absorbed refers to at least 0.01% liquid contents within the porous material. According to some embodiments, partially absorbed refers to at least 0.5% liquid contents within the porous material. According to some embodiments, partially absorbed refers to at least 0.1% liquid contents within the porous material. According to some embodiments, partially absorbed refers to at least 1% liquid contents within the porous material. According to some embodiments, partially absorbed refers to not more than 5% liquid contents within the porous material.

According to some embodiments, evaporation heater 120 has a total resistance in the range of 0.10Ω to 0.20Ω. According to some embodiments, evaporation heater 120 has a total resistance in the range of 0.12Ω to 0.17Ω. According to some embodiments, evaporation heater 120 has a total resistance in the range of 0.13Ω to 0.16Ω. According to some embodiments, evaporation heater 120 has a total resistance in the range of 0.14Ω to 0.15Ω. According to some embodiments, evaporation heater 120 has a total resistance of about 0.13Ω.

According to some embodiments, distal flat side of evaporation heater 120 has a projected area of not more than 75 mm². According to some embodiments, distal flat side of evaporation heater 120 has a projected area of not more than 100 mm². According to some embodiments, distal flat side of evaporation heater 120 has a projected area of not more than 150 mm². According to some embodiments, distal flat side of evaporation heater 120 has a projected area of not more than 200 mm². According to some embodiments, distal flat side of evaporation heater 120 has a projected area of not more than 250 mm². According to some embodiments, distal flat side of evaporation heater 120 has a projected area of not more than 300 mm². According to some embodiments, distal flat side of evaporation heater 120 has a projected area of not more than 325 mm². According to some embodiments, distal flat side of evaporation heater 120 has a projected area of not more than 350 mm². According to some embodiments, distal flat side of evaporation heater 120 has a projected area of not more than 375 mm². According to some embodiments, distal flat side of evaporation heater 120 has a projected area of not more than 400 mm².

According to some embodiments, distal flat side of evaporation heater 120 has a projected area of at least 10 mm². According to some embodiments, distal flat side of evaporation heater 120 has a projected area of at least 15 mm². According to some embodiments, distal flat side of evaporation heater 120 has a projected area of at least 20 mm². According to some embodiments, distal flat side of evaporation heater 120 has a projected area of at least 25 mm². According to some embodiments, distal flat side of evaporation heater 120 has a projected area of at least 30 mm². According to some embodiments, distal flat side of evaporation heater 120 has a projected area of at least 35 mm². According to some embodiments, distal flat side of evaporation heater 120 has a projected area of at least 40 mm².

According to some embodiments, distal flat side of evaporation heater 120 has a projected area in the range of 25-75 mm². According to some embodiments, distal flat side of evaporation heater 120 has a projected area in the range of 30-70 mm². According to some embodiments, distal flat side of evaporation heater 120 has a projected area in the range of 35-65 mm². According to some embodiments, distal flat side of evaporation heater 120 has a projected area in the range of 40-60 mm². According to some embodiments, distal flat side of evaporation heater 120 has a projected area in the range of 45-55 mm². According to some embodiments, distal flat side of evaporation heater 120 has a projected area of about 50 mm². According to some embodiments, distal flat side of evaporation heater 120 has a projected area of about 45 mm².

According to some embodiments, evaporation heater 120 is configured to enable small diameter droplets to pass through the structure thereof and to obstruct large diameter droplets from passing through the material thereof.

A resistive porous material, such as the material for constructing evaporation heater 120, can be produced by curing so called conductive inks in which the degree of conductivity (or resistivity) are controlled by the ratio of metallic to ceramic micro and nanoparticles. Porous structures can be directly obtained by choosing certain geometries and sizes of the nanoparticles. Alternatively, a porous structure can be achieved by—initially obtaining a non-porous structure and subsequently subjecting it to controlled etching.

According to some embodiments, the alloy is a nicotine-passivated alloy. According to some embodiments, the metal is a nicotine-passivated metal. According to some embodiments, the Nichrome is a nicotine-passivated.

It is to be understood that embodiment referring to second trigger 150 and flow or pressure sensor 152 apply to any electronic cigarettes 100 as presented herein. Specifically, embodiments referring to second trigger 150 and flow or pressure sensor 152 apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

According to some embodiments, electronic cigarette 100 further comprises a second trigger 150, configured to at least trigger activation or deactivation of at least one of evaporation heater 120 and liquid deposition mechanism 160.

According to some embodiments, second trigger 150 is configured to generate a variable second trigger activation signal, varying in at least one of: amplitude, wavelength or frequency of the signals. According to some embodiments, processing unit 190 is configured to provide varying activation signals to liquid deposition mechanism 160, thereby controlling various parameters of liquid deposition mechanism 160 as a function of the second trigger activation signals generated by second trigger 150. Varying activation signals of liquid deposition mechanism 160 may include, but are not limited to variations in the amount of liquid drawn by liquid deposition mechanism 160 towards evaporation heater 120, or to rate of liquid transfer from liquid deposition mechanism 160 towards evaporation heater 120.

According to some embodiments, second trigger 150 is further configured to generate a deactivation signal. According to some embodiments, processing unit 190 is configured to deactivate liquid deposition mechanism 160 upon receiving second trigger deactivation signal from second trigger 150. According to some embodiments, processing unit 190 is configured to activate and deactivate liquid deposition mechanism 160 intermittently, such that discrete amounts of the liquid are delivered to evaporation heater 120. According to some embodiments, processing unit 190 is configured to alternately activate and deactivate liquid deposition mechanism 160, such that there is no continuous delivery of the liquid to evaporation heater 120.

According to some embodiments, second trigger 150 comprises a flow sensor or a pressure sensor 152, configured to detect the flow or the pressure, respectively, in electronic cigarette 100, and to generate signals indicative thereof. According to some embodiments, flow sensor or pressure sensor 152 comprises a differential pressure sensor. According to some embodiments, pressure sensor 152 is positioned within actuator 114.

According to some embodiments, the signals produced by flow or pressure sensor 152 are received by processing unit 190. According to some embodiments, processing unit 190 is configured to receive the flow or pressure signals. According to some embodiments, the flow or pressure signals are indicative of the usage of electronic cigarette 100. For example, upon inhalation of a user from electronic cigarette 100 through outlet 110, the pressure drops and a reduced pressure signal is sent from flow or pressure sensor 152 to processing unit 190. According to some embodiments, processing unit 190 is configured to receive the flow or pressure signals and to activate at least one of evaporation heater 120 and liquid deposition mechanism 160 in response thereto. According to some embodiments, processing unit 190 is configured to receive the flow or pressure signals and to deactivate at least one of evaporation heater 120 and liquid deposition mechanism 160 in response thereto. For example continuing the previous example, upon the user stopping to inhale through outlet 110, the pressure within electronic cigarette 100 will rise again and a respective pressure signal will be sent from flow or pressure sensor 152 to processing unit 190. In response processing unit 190 will terminate the activation of liquid deposition mechanism 160 and evaporation heater 120, according to some embodiments.

As shown in FIG. 2 and detailed herein, actuator 114 and cartridge 106 are reversibly connectable, according to some embodiments.

According to some embodiments, cartridge 106 comprises liquid container 162. According to some embodiments, liquid container 162 is contained within cartridge internal compartment 108 of cartridge 106.

According to some embodiments, cartridge 106 comprises outlet 110. According to some embodiments, outlet 110 is formed on cartridge housing 102 of cartridge 106.

According to some embodiments, cartridge 106 comprises evaporation heater 120. According to some embodiments, evaporation heater 120 is contained within cartridge internal compartment 108 of cartridge 106.

According to some embodiments, cartridge 106 comprises support 122. According to some embodiments, support 122 is connected to cartridge housing 102 of cartridge 106.

According to some embodiments, cartridge 106 comprises liquid drawing element 164. According to some embodiments, liquid drawing element 164 is connected to cartridge housing 102 of cartridge 106.

According to some embodiments, cartridge 106 further comprises at least one cartridge opening 112 allowing passage there through of solenoid plunger head 172 from actuator 114 to cartridge internal compartment 108. According to some embodiments, cartridge 106 further comprises at least one cartridge opening 112 allowing fluid communication between actuator 114 and cartridge 106. Specifically, according to some embodiments, fluid communication between cartridge 106 and actuator 114 may be required because (a) second trigger 150 may be a pressure sensor (e.g. sensor 152); (b) sensor 152 is located in actuator 114; and (c) sensor 152 senses pressure or flow changes correlating with a user inhalation through outlet 110, which is part of cartridge 106.

According to some embodiments, cartridge 106 comprises cartridge power coupling 196. According to some embodiments, cartridge power coupling 196 is contained within cartridge internal compartment 108 of cartridge 106.

According to some embodiments, cartridge 106 comprises evaporation heater electric contact 132 and cartridge electric contacts 134. According to some embodiments, evaporation heater electric contact 132 and cartridge electric contacts 134 are contained within cartridge internal compartment 108 of cartridge 106.

According to some embodiments, actuator 114 comprises solenoid actuator 170 and liquid deposition mechanism housing 178.

According to some embodiments, actuator 114 comprises flow or pressure sensor 152.

According to some embodiments, actuator 114 comprises power source compartment 192.

According to some embodiments, actuator 114 comprises processing unit assembly 173.

According to some embodiments, actuator 114 comprises compartment of processing unit assembly 174.

According to some embodiments, actuator 114 comprises processing unit 190.

According to some embodiments, cartridge 106 is intended to be disposable and for use until the formulation contained therein is consumed whereas actuator 114 is durable and after consumption of the liquid contained in a first cartridge 106, a second cartridge 106 may be mounted on actuator 114 for a further sequence of aerosolizations.

According to some embodiments, electronic cigarette 100 further comprises a communication element (not shown) configured to enable wireless communication of electronic cigarette 100 with servers, databases and personal devices (e.g. computers, mobile phones) among others.

According to some embodiments, the communication element provides wireless communication through Bluetooth, Wi-Fi, ZigBee and/or Z-wave.

Reference is now made to FIG. 4 and FIG. 5. FIG. 4 and FIG. 5 constitute schematic illustration of an electronic cigarette 100, according to some embodiments. Electronic cigarette 100 comprises a cartridge 106 comprising a cartridge housing 102 and a cartridge internal compartment 108. Electronic cigarette 100 further comprises an actuator 114 comprising an actuator housing 104. Electronic cigarette 100 further comprises an outlet 110, an evaporation heater 120, a first trigger 140, a liquid deposition mechanism 160 and a processing unit 190.

Electronic cigarette 100 as described in FIGS. 4-5 differs from electronic cigarette 100 as described in FIGS. 1-3 mainly in the design of liquid deposition mechanism 160. Other embodiments, such embodiment referring to evaporation heater 120, actuator 114 and processing unit 190, similarly apply, when applicable, to each of FIGS. 1-5.

According to some embodiments, outlet 110 is formed on cartridge housing 102. According to some embodiments, electronic cigarette 100 is configured to produce an aerosol 166, and outlet 110 is configured to deliver aerosol 166 out of electronic cigarette 100. It is to be understood that the objective of electronic cigarettes is generally to produce an aerosol, and to deliver it through the outlet and/or mouthpiece of the electronic cigarette, through a mouth of an electronic cigarette user to the respiratory system of the user.

According to some embodiments, outlet 110 is connected to a mouthpiece (not shown). According to some embodiments, outlet 110 is mechanically connected to a mouthpiece. According to some embodiments, the mouthpiece is detachable.

According to some embodiments, evaporation heater 120 is accommodated within cartridge internal compartment 108.

Generally, electronic cigarettes, including electronic cigarette 100 have an elongated shape, as depicted in FIGS. 1-6, 30A-B and 28A-C. Within the context of this specification the term “longitudinal” refers to the direction of elongation of electronic cigarette 100. The term “longitudinal axis” refers to the linear axis along the longitudinal direction.

Generally, during operation of electronic cigarette 100, liquid deposition mechanism 160 delivers a discrete, known volume of liquid, or a plurality of discrete, known volumes of liquid, intermittently to evaporation heater 120. Evaporation heater 120 is heated to an elevated temperature, which rapidly evaporates the discrete volume of liquid and generates aerosol 166 therefrom, according to some embodiments.

The intermittent nature of liquid delivery from liquid deposition mechanism 160 to evaporation heater 120 has benefits, especially when aerosolizing aqueous formulations, and is achieved using a two-state liquid deposition mechanism 160, according to some embodiments

Specifically, according to some embodiments, in a first state of electronic cigarette 100, liquid deposition mechanism 160 is spaced apart from evaporation heater 120, such that liquid is not deposited onto evaporation heater 120, when electronic cigarette 100 is in the first state of operation.

In a second state of electronic cigarette 100, according to some embodiments, liquid deposition mechanism 160 is delivering a discrete volume of liquid onto evaporation heater 120, and the discrete volume of liquid is evaporated and subsequently aerosolized, due to evaporation heater 120 being in an elevated evaporation temperature. In the second state of electronic cigarette 100, liquid deposition mechanism 160 may be spaced apart from evaporation heater 120 and deposit liquid thereon from distance, according to some embodiments, and as detailed with respect to FIGS. 4-5.

According to some embodiments, evaporation heater 120 is located longitudinally between outlet 110 and liquid deposition mechanism 160. Specifically, as defined above with respect to directions, evaporation heater 120 is located above liquid deposition mechanism 160, and outlet 110 is located above evaporation heater 120. Therefore, upon operation of electronic cigarette 100 from the first state to the seconds state, liquid deposition mechanism 160 deposits the discrete volume of liquid on the bottom of evaporation heater 120, and vapor is released from the top of evaporation heater 120.

According to some embodiments, evaporation heater 120 is flat and comprises a first surface facing outlet 110 and a second surface facing liquid deposition mechanism 160.

According to some embodiments, electronic cigarette 100 comprises compartment of processing unit assembly 173, accommodated within actuator 114. According to some embodiments, compartment of processing unit assembly 173 accommodates comprises processing unit assembly 174. According to some embodiments, comprises processing unit assembly 174 comprises processing unit 190. According to some embodiments, electronic cigarette 100 comprises processing unit 190, accommodated within actuator 114.

Compartment of processing unit assembly 173 is shown in FIGS. 4-5. The contents of compartment of processing unit assembly 173, including processing unit 190 are elaborated when referring to FIGS. 12A and 12B.

According to some embodiments, processing unit 190 is configured to receive signals from first trigger 140. According to some embodiments, first trigger 140 is configured to generate at least a first trigger activation signal. According to some embodiments, evaporation heater 120 is configured to generate heat when first trigger 140 generates the first trigger activation signal.

According to some embodiments, liquid deposition mechanism 160 is configured to control the operation of evaporation heater 120. According to some embodiments, processing unit 190 is configured to activate evaporation heater 120 upon receiving first trigger activation signal from first trigger 140. According to some embodiments, processing unit 190 is configured to deactivate at least one heating element.

According to some embodiments, processing unit 190 is configured to control operation of liquid deposition mechanism 160. According to some embodiments, processing unit 190 is configured to control operation of liquid deposition mechanism 160, such that liquid deposition mechanism 160 delivers a discrete volume of liquid to evaporation heater 120. According to some embodiments, processing unit 190 is configured to operate liquid deposition mechanism 160 to perform a transition from the first state to the second state of electronic cigarette 100.

The term “transition” as used with respect to electronic cigarette 100 and liquid deposition mechanism 160 of FIGS. 4-5, is not limited to movement. This term may further encompass functional transition from a first state to a second state as follows: liquid deposition mechanism 160 and evaporation heater 120 are spaced apart and liquid deposition mechanism 160 is not operated to deposit liquids onto evaporation heater 120 (first state); and evaporation heater 120 and liquid deposition mechanism 160 remain in the same relative positions, but liquid deposition mechanism 160 is operated to deposit liquid onto evaporation heater 120 (second state).

According to some embodiments, processing unit 190 is configured to operate liquid deposition mechanism 160 to perform a transition from the second state to the first state of electronic cigarette 100. According to some embodiments, processing unit 190 is configured to operate liquid deposition mechanism 160 to perform a transition from the first state to the second state and vise versus, consecutively, to provide a discrete volume of liquid from liquid deposition mechanism 160 to evaporation heater 120. According to some embodiments, processing unit 190 is configured to operate liquid deposition mechanism 160 to perform the following sequence of operations consecutively:
(a) a transition of liquid deposition mechanism 160 from the first state to the second state of electronic cigarette 100;
(b) maintenance of liquid deposition mechanism 160 in the second state for a predetermined period of deposition time; wherein during the predetermined period of deposition time, liquid deposition mechanism 160 is configured to deliver a discrete volume of liquid to evaporation heater 120; and
(c) a transition of liquid deposition mechanism 160 from the second state to the first state of electronic cigarette 100.

As explained with respect to the term “transition” as used pertaining electronic cigarette 100 and liquid deposition mechanism 160 of FIGS. 4-5, the phrase “maintenance of liquid deposition mechanism 160 in the second state for a predetermined period of deposition time” means that liquid deposition mechanism 160 is delivering liquid to evaporation heater 120 throughout the predetermined period of time.

According to some embodiments, operation (b) is the only operation in which liquid deposition mechanism 160 is configured to deliver a liquid to evaporation heater 120.

According to some embodiments, processing unit 190 is configured to perform the sequence of operations a plurality of times upon receiving the first activation signal.

According to some embodiments, processing unit 190 is configured to activate liquid deposition mechanism 160 upon receiving first trigger activation signal from first trigger 140. According to some embodiments, processing unit 190 is configured to deactivate liquid deposition mechanism 160.

According to some embodiments, liquid deposition mechanism 160 comprises a liquid deposition mechanism housing 178, a liquid container 162, a liquid drawing element 164 and an ultrasonic mechanism comprising a piezo disc 180.

FIG. 5 constitutes a cross sectional view of electronic cigarette 100 in the second state of operation, when actuator 114 and 106 are separated, and FIG. 4 constitutes a cross sectional view of electronic cigarette 100 in the second state of operation, when actuator 114 and 106 are joined.

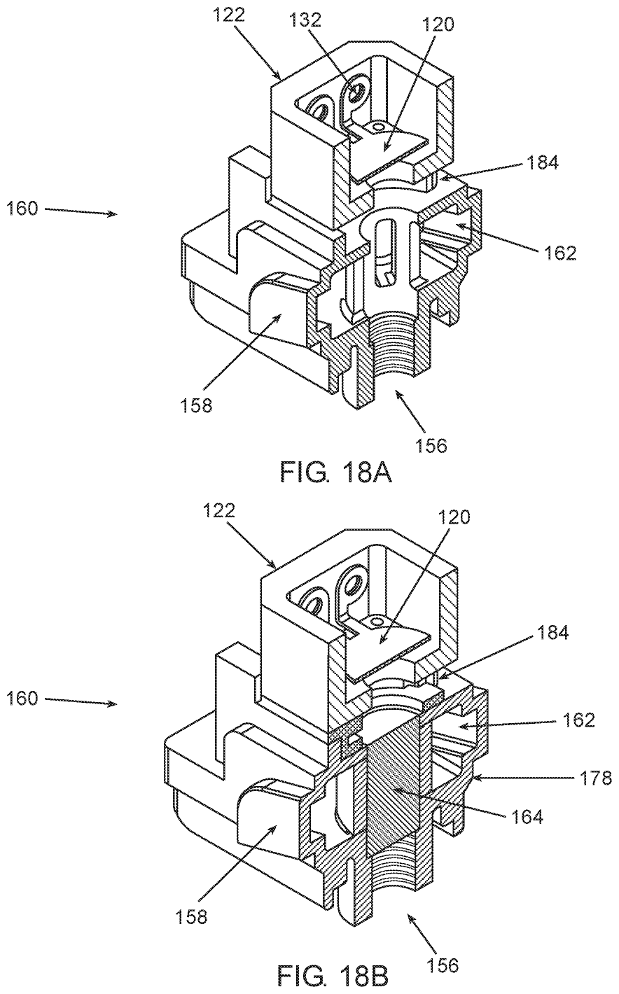

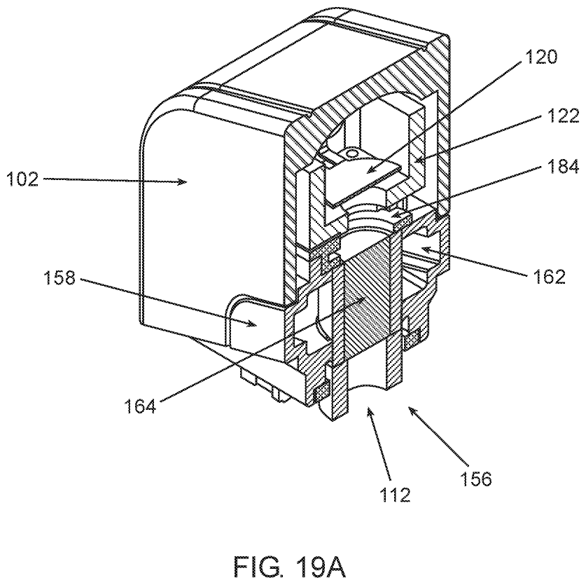

Liquid container 162 is accommodated within cartridge internal compartment 108 of cartridge 106 and is configured to contain the liquid therein. FIG. 14A and FIGS. 18A-B are cross sectional views of liquid deposition mechanism 160, which enable view of liquid container 162.

Specifically, as shown in FIG. 14A and FIGS. 18A-B, liquid drawing element 164 is in contact with liquid container 162, according to some embodiments. According to some embodiments, liquid container 162 and liquid drawing element 164 are positioned in contact, such that delivery of liquids from liquid container 162 to liquid drawing element 164 is enabled.

In contrast with the discrete volume of liquid, which are small and typically sufficient for a single inhalation of aerosol 166 by a user of 100, liquid container 162 is configured to contain bulk amount of the liquid formulation, wherein only small discrete volume(s) of the liquid are evaporated during the operation of electronic cigarette 100.

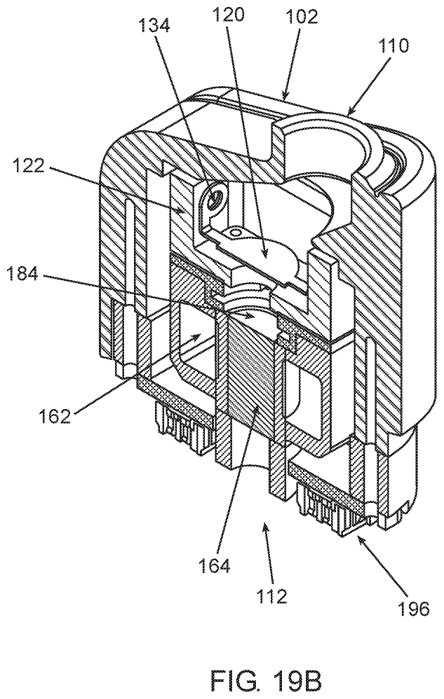

According to some embodiments, liquid container 162 is surrounding liquid drawing element 164, such that transfer of liquid contained therein of liquid drawing element 164 is enabled through the circumference of liquid drawing element 164 (see FIGS. 18A, 18B, 19A and 19B).

According to some embodiments, liquid drawing element 164 is fluidly attached to liquid container 162. According to some embodiments, liquid drawing element 164 is in constant contact with liquid container 162. According to some embodiments, liquid drawing element 164 is partially accommodated within liquid container 162.

According to some embodiments, liquid is provided in liquid container 162 for deliverance towards evaporation heater 120 via liquid drawing element 164.

According to some embodiments, liquid drawing element 164 comprises a material that is capable of incorporating, taking in, drawing in or soaking liquids, and upon applying physical pressure thereto or being in contact with another material, release a portion or the entire amount/volume of the absorbed liquid.

According to some embodiments, liquid drawing element 164 is affixed to at least one of cartridge housing 102, cartridge internal compartment 108 and liquid container 162. According to some embodiments, liquid drawing element 164 is affixed to at least one of cartridge housing 102, cartridge internal compartment 108 and liquid container 162, such that liquid drawing element 164 is in contact with liquid container 162 and capable of withdrawing liquid therefrom. FIGS. 19A and 19B depicts a configuration, in which liquid drawing element 164 is connected to cartridge housing 102.

According to some embodiments, liquid drawing element 164 is configured to absorb liquid in an amount which is at least 100% of its weight. According to some embodiments, liquid drawing element 164 is configured to absorb liquid in an amount which is at least 50% of its weight.

According to some embodiments, liquid drawing element 164 is fabricated such that contact of liquid drawing element 164 with evaporation heater 120 for said the predetermined period of deposition time results in the delivery of a discrete volume of liquid to evaporation heater 120. According to some embodiments, liquid drawing element 164 is fabricated such that contact of liquid drawing element 164 with evaporation heater 120 for said the predetermined period of deposition time results in the delivery of a thin layer of liquid to evaporation heater 120. According to some embodiments, the thin layer of liquid has thickness in the range of 0.1 mm to 0.5 mm.

According to some embodiments, liquid drawing element 164 comprises cloth, wool, felt, sponge, foam, cellulose, yarn, microfiber or a combination thereof, having high tendency to absorb aqueous solutions. Each possibility represents a separate embodiment. According to some embodiments, the sponge is an open cell sponge. According to some embodiments, the sponge is a closed cell sponge.

According to some embodiments, liquid drawing element 164 comprises fabric. Specifically, fibrous and/or woven fabric, such as a wick, is a hydrophilic and liquid absorbing material, which may be used as the stationary liquid absorbing element(s), according to some embodiments.

According to some embodiments, liquid drawing element 164 is a hydrophilic liquid drawing element. According to some embodiments, liquid drawing element 164 is a hydrophilic sponge.

According to some embodiments, a liquid deposition mechanism, such as liquid deposition mechanism 160 described in FIGS. 4-5, in which liquid drawing element 164 is intermittently providing discrete volumes of liquid to evaporation heater 120 from a distance, during the second state of operation of electronic cigarette 100 is preferably used with liquid solutions, such as aqueous solutions of nicotine or liquid solutions of cannabinoids.

According to some embodiments, liquid drawing element 164 is essentially stationary during both the first state of electronic cigarette 100 and the second state of electronic cigarette 100. According to some embodiments, the distance between liquid drawing element 164 and evaporation heater 120 is substantially constant during both the first state of electronic cigarette 100 and the second state of electronic cigarette 100.

According to some embodiments, liquid deposition mechanism 160 includes liquid drawing element 164, liquid deposition mechanism housing 178, piezo disc 180 and a piezo slot 184.

FIG. 5 constitutes a view in which actuator 114 and cartridge 106 are separated.

Liquid deposition mechanism housing 178 is located inside cartridge 106 and is configured to accommodate piezo disc 180. According to some embodiments, liquid deposition mechanism housing 178 comprises piezo slot 184, which is configured to accommodate piezo disc 180. According to some embodiments, liquid deposition mechanism housing 178 is connected to actuator housing 104. According to some embodiments, piezo disc 180 is connected to liquid deposition mechanism housing 178.

According to some embodiments, liquid deposition mechanism housing 178 is rigidly attached to actuator housing 104. According to some embodiments, piezo disc 180 is affixed to piezo slot 184, such that unintentional displacement of piezo disc 180 upwards or downward in the longitudinal direction is prevented. According to some embodiments, piezo slot 184 is attached to piezo disc 180, such that displacement of piezo disc 180 upwards or downward in the longitudinal direction is prevented. According to some embodiments, piezo disc 180 is attached to piezo slot 184 such that unintentional displacement of piezo disc 180 in a non-longitudinal direction is prevented. According to some embodiments, piezo slot 184 is attached to piezo disc 180, such that displacement of piezo disc 180 in a non-longitudinal direction is prevented.

According to some embodiments, liquid deposition mechanism 160 comprises a liquid drawing element positioning compartment 156. According to some embodiments, liquid drawing element positioning compartment 156 is formed within liquid deposition mechanism housing 178. According to some embodiments, liquid drawing element positioning compartment 156 is positioned below liquid drawing element 164.

In general, liquid drawing element positioning compartment 156 comprises a compartment for installing a positioning mechanism (not shown) for proper positioning of liquid drawing element 164 in the longitudinal axis, according to some embodiments. According to some embodiments, the positioning mechanism is configured to cause a contact between piezo disc 180 and liquid drawing element 164. Specifically, according to some embodiments, liquid drawing element 164 comprises a top surface in contact with piezo disc 180 and a bottom surface in contact with the positioning mechanism. According to some embodiments, the positioning mechanism is configured to apply pressure on the bottom surface of liquid drawing element 164, such that the top surface of liquid drawing element 164 contacts piezo disc 180. According to some embodiments, the applied pressure is upwards in the longitudinal direction. According to some embodiments, the positioning mechanism is configured to apply pressure on the bottom surface of liquid drawing element 164, such that the top surface of liquid drawing element 164 is pressed against piezo disc 180.

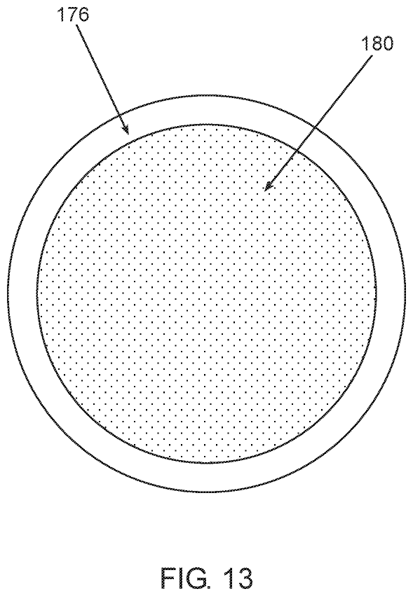

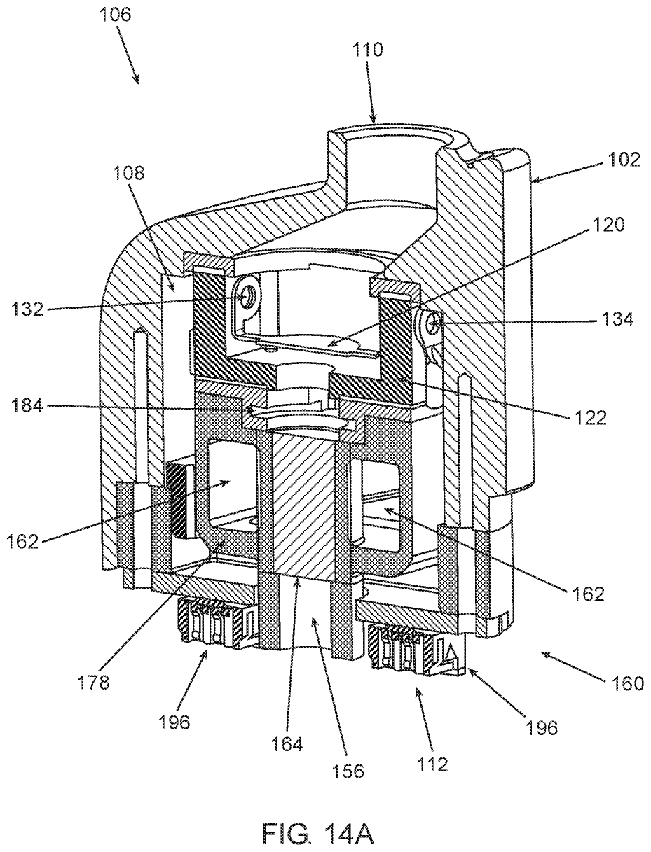

Reference is made to FIG. 13. According to some embodiments, a piezo gasket 176 is accommodated within piezo slot 184, and is configured to fasten piezo disc 180 to piezo slot 184. According to some embodiments, piezo gasket 176 comprises a silicone gasket. According to some embodiments, piezo gasket 176 comprises a rubber gasket. According to some embodiments, piezo gasket 176 comprises an O-ring.

According to some embodiments, piezo disc 180 is screwed to piezo slot 184.

According to some embodiments, piezo disc 180 is configured to convert electric current to mechanic stress. According to some embodiments, piezo disc 180 is configured to convert electric current to vibrations. According to some embodiments, piezo disc 180 is configured to convert electric current to vibrations having resonant frequency, which creates mist from liquid formulations. According to some embodiments, piezo disc 180 is configured to convert electric current to vibrations having resonant frequency, which creates mist from aqueous formulations. Thus, according to some embodiments, upon driving sufficient current through piezo disc 180 and upon depositing liquid thereon, it creates mist of the liquid.

According to some embodiments, piezo disc 180 has piezo resonant frequency in the range of 100 KHz-10 MHz. According to some embodiments, piezo disc 180 has piezo resonant frequency in the range of 100-250 KHz. According to some embodiments, piezo disc 180 has piezo resonant frequency in the range of 125-225 KHz. According to some embodiments, piezo disc 180 has piezo resonant frequency in the range of 140-210 KHz. According to some embodiments, piezo disc 180 has piezo resonant frequency in the range of 150-200 KHz. According to some embodiments, piezo disc 180 has piezo resonant frequency in the range of 165-195 KHz. According to some embodiments, piezo disc 180 has piezo resonant frequency in the range of 175-185 KHz.

According to some embodiments, piezo disc 180 has capacitance in the range of 700-2000 pF. According to some embodiments, piezo disc 180 has capacitance in the range of 700-1700 pF. According to some embodiments, piezo disc 180 has capacitance in the range of 800-1600 pF. According to some embodiments, piezo disc 180 has capacitance in the range of 950-1450 pF.

According to some embodiments, piezo disc 180 has harmonic impedance of not more than 500 Ohm. According to some embodiments, piezo disc 180 has harmonic impedance of not more than 450 Ohm. According to some embodiments, piezo disc 180 has harmonic impedance of not more than 400 Ohm. According to some embodiments, piezo disc 180 has harmonic impedance of not more than 350 Ohm.

According to some embodiments, piezo disc 180 has piezoelectric coefficient D₃₃ of not more than 450 C/N. According to some embodiments, piezo disc 180 has piezoelectric coefficient D₃₃ of not more than 400 C/N. According to some embodiments, piezo disc 180 has piezoelectric coefficient D₃₃ of not more than 350 C/N. According to some embodiments, piezo disc 180 has piezoelectric coefficient D₃₃ of not more than 300 C/N.

According to some embodiments, piezo disc 180 is made of a metal. According to some embodiments, piezo disc 180 is made of stainless steel. According to some embodiments, piezo disc 180 is made of SUS304 stainless steel.

According to some embodiments, piezo disc 180 is configured to receive electric current and to generate mist 182 from liquid upon receiving the electric current. According to some embodiments, piezo disc 180 comprises a top flat surface facing evaporation heater 120 and a bottom flat surface in contact with liquid drawing element 164. According to some embodiments, the bottom flat surface of piezo disc 180 is in contact with liquid drawing element 164 during both the first state and the second state of electronic cigarette 100. According to some embodiments, piezo disc 180 is a perforated disc. According to some embodiments, piezo disc 180 is a perforated disc, such that fluids may pass therethrough. According to some embodiments, the bottom surface of piezo disc 180 is in contact with liquid contained in liquid drawing element 164 during both the first state and the second state of electronic cigarette 100. According to some embodiments, upon application of electric current through piezo disc 180, piezo disc 180 converts liquid in contact with the bottom surface there to mist 182, which is released through the perforations of piezo disc 180 from the top surface of piezo disc 180. According to some embodiments, mist 182 is released from the top surface of piezo disc 180 longitudinally upwards, such that it forms a discrete volume of liquid on the bottom surface of evaporation heater 120.

According to some embodiments, processing unit 190 is configured to control piezo disc 180, by providing current thereto. According to some embodiments, processing unit 190 is configured to control piezo disc 180 such that piezo disc 180 generates, intermittently a plurality of mists 182 at a predetermined rate. According to some embodiments, processing unit 190 is configured to control piezo disc 180 such that piezo disc 180 generates, intermittently a plurality of mists 182 at a rate controlled by processing unit 190.

According to some embodiments, processing unit 190 is configured to control piezo disc 180. According to some embodiments, processing unit 190 is configured to pass current to piezo disc 180. According to some embodiments, upon receiving the electric current, piezo disc 180 is configured to generate mists 182, intermittently at a controlled rate, wherein processing unit 190 is configured to control the controlled rate. According to some embodiments, processing unit 190 is configured to pass variable current to piezo disc 180 wherein the variable current is dictating the controlled rate. According to some embodiments, processing unit 190 is configured to pass variable current to piezo disc 180 wherein the variable current is dictating the mass of mist 182.

According to some embodiments, in the first state of electronic cigarette 100, piezo disc 180 is deactivated. According to some embodiments, in the first state of electronic cigarette 100, current is not driven through piezo disc 180. According to some embodiments, in the first state of electronic cigarette 100, processing unit 190 does not provide current to piezo disc 180. According to some embodiments, in the first state of electronic cigarette 100, piezo disc 180 does not generate mist 182.

According to some embodiments, in the second state of electronic cigarette 100, piezo disc 180 is activated. According to some embodiments, in the second state of electronic cigarette 100, current is driven through piezo disc 180. According to some embodiments, in the second state of electronic cigarette 100, processing unit 190 provide current to piezo disc 180. According to some embodiments, in the second state of electronic cigarette 100, piezo disc 180 generates mist 182.

According to some embodiments, processing unit 190 is configured is activate and deactivate piezo disc 180 intermittently at a controlled rate, such that a plurality of mists 182 is delivered intermittently to evaporation heater 120.

According to some embodiments, processing unit 190 is configured to alternately operate piezo disc 180, such that piezo disc 180 delivers discrete volumes of liquid to evaporation heater 120, alternately.

According to some embodiments, liquid deposition mechanism 160 is configured to transfer liquid to evaporation heater 120. According to some embodiments, liquid deposition mechanism 160 is configured to deliver a thin film or layer of the liquid to evaporation heater 120. According to some embodiments, liquid deposition mechanism 160 is configured to deliver a film liquid to evaporation heater 120 having a thickness in the range of 0.1 mm to 3 mm. According to some embodiments, the film has a thickness in the range of 0.1 mm to 2 mm. According to some embodiments, the film has a thickness in the range of 0.5 mm to 2 mm. According to some embodiments, the film has a thickness in the range of 0.75 mm to 1.5 mm.

According to some embodiments, liquid deposition mechanism 160 is configured to deliver a discrete volume of liquid to evaporation heater 120, wherein the discrete volume of liquid has a volume in the range of 2 μL to 100 μL. According to some embodiments, the discrete volume of liquid has a volume in the range of 3 μL to 50 μL. According to some embodiments, the discrete volume of liquid has a volume in the range of 4 μL to 45 μL. According to some embodiments, the discrete volume of liquid has a volume in the range of 5 μL to 40 μL. According to some embodiments, the discrete volume of liquid has a volume in the range of 6 μL to 35 μL. According to some embodiments, the discrete volume of liquid has a volume in the range of 7 μL to 30 μL. According to some embodiments, the discrete volume of liquid has a volume in the range of 8 μL to 28 μL. According to some embodiments, the discrete volume of liquid has a volume in the range of 9 μL to 25 μL. According to some embodiments, the discrete volume of liquid has a volume in the range of 10 μL to 20 μL.

According to some embodiments, liquid deposition mechanism 160 is configured to transfer discrete volume of liquid to evaporation heater 120. According to some embodiments, the liquid comprises a nicotine formulation. According to some embodiments, the nicotine formulation is an aqueous nicotine formulation. According to some embodiments, the nicotine formulation is an aqueous nicotine solution. According to some embodiments, the aqueous nicotine formulation comprises from 1% to 5% nicotine w/w. According to some embodiments, the aqueous nicotine formulation comprises from 2% to 4% nicotine w/w. According to some embodiments, liquid container 162 contains the liquid.

According to some embodiments, the liquid comprises a cannabinoid formulation. According to some embodiments, the cannabinoid formulation is an aqueous cannabinoid formulation. According to some embodiments, the cannabinoid formulation is an aqueous cannabinoid solution. According to some embodiments, the cannabinoid formulation is an aqueous cannabinoid solution having pH higher than 8. According to some embodiments, the pH is higher than 9. According to some embodiments, the pH is higher than 10. According to some embodiments, the pH is higher than 10.5. According to some embodiments, the aqueous cannabinoid formulation comprises from 1% to 10% tetrahydrocannabinolic acid (THCA) basic salt w/w. According to some embodiments, the aqueous cannabinoid formulation comprises from 2% to 8% THCA basic slat w/w. According to some embodiments, the liquid comprises the cannabinoid composition disclosed herein. According to some embodiments, the liquid is the cannabinoid composition disclosed herein. According to some embodiments, liquid container 162 contains the liquid.

According to some embodiments, first trigger 140 may be a touch user interface, according to some embodiments. According to some embodiments, the user interface may provide options to a user for determining parameters by which processing unit 190 controls liquid deposition mechanism 160 and/or evaporation heater 120. According to some embodiments, the touch user interface is configured to provide to an electronic cigarette 100 user at least two sensorial options. According to some embodiments, upon selecting each of the at least two sensorial options, at least one control parameter of processing unit 190 over liquid deposition mechanism 160 are executed. According to some embodiments, the at least one control parameter is selected from fluid deposition frequency and fluid deposition duty cycle.

According to some embodiments, the fluid deposition frequency is in the range of 0.5 Hz to 100 Hz. According to some embodiments, the fluid deposition frequency is in the range of 0.5 Hz to 50 Hz. According to some embodiments, the fluid deposition frequency is in the range of 0.75 Hz to 40 Hz. According to some embodiments, the fluid deposition frequency is in the range of 1 Hz to 30 Hz. According to some embodiments, the fluid deposition frequency is in the range of 1.5 Hz to 25 Hz. According to some embodiments, the fluid deposition frequency is in the range of 2 Hz to 20 Hz. According to some embodiments, the fluid deposition frequency is in the range of 2 Hz to 10 Hz.

According to some embodiments, the duty cycle is in the range of 5% to 80%. According to some embodiments, the duty cycle is in the range of 7% to 70%. According to some embodiments, the duty cycle is in the range of 10% to 60%. According to some embodiments, the duty cycle is in the range of 12% to 50%. According to some embodiments, the duty cycle is in the range of 14% to 40%. According to some embodiments, the duty cycle is in the range of 15% to 35%. According to some embodiments, the duty cycle is in the range of 20% to 30%.

The phrase “fluid deposition frequency” refers to the number of times in which liquid deposition mechanism 160 deposits discrete volume of liquid onto evaporation heater 120 per time unit. Alternatively, the phrase “fluid deposition frequency” refers to the number of times in which electronic cigarette 100 transforms from the first state to the second state of action per time unit.

The phrase “fluid deposition frequency” refers to the time ratio between the first state and the second state of electronic cigarette 100. As detailed herein during the second state, a discrete volume of liquid is delivered to evaporation heater 120, and during the first state liquid is not delivered to evaporation heater 120. Thus, the phrase “fluid deposition frequency” refers to the relative duration in which evaporation heater 120 is being deposited with liquid.

According to some embodiments, upon selecting each of the at least two sensorial options, at least one control parameter of processing unit 190 over evaporation heater 120 are executed. According to some embodiments, the at least one control parameter comprises evaporation heater 120 threshold temperature. As detailed herein, the threshold temperature is the temperature above which, processing unit 190 stops driving current- or reducing the current driven to evaporation heater 120, for its heating.

According to some embodiments, first trigger 140 is further configured to generate a deactivation signal, such that processing unit 190 is configured to deactivate both evaporation heater 120 and piezo disc 180 upon receiving first trigger deactivation signal from first trigger 140.

Thus, according to some embodiments, processing unit 190 is configured to regulate the temperature of evaporation heater 120 in the range of 95° C. to 400° C., through control of the operation of both evaporation heater 120 and liquid deposition mechanism 160. According to some embodiments, processing unit 190 is configured to regulate the temperature of evaporation heater 120 below 400° C., below 350° C., or below 330° C., through control of the operation of liquid deposition mechanism 160 and/or evaporation heater 120. According to some embodiments, processing unit 190 is configured to receive at least one operation signal and to control operation of liquid deposition mechanism 160.

According to some embodiments, processing unit 190 is configured to regulate the temperature of evaporation heater 120 above the nicotine-water azeotropic temperature of 99.5° C. According to some embodiments, the regulation entails providing variable current to piezo disc 180 as detailed above. Specifically, it is to be understood that deposition of liquid over evaporation heater 120 effects its temperature.

As shown in FIG. 5 and detailed herein, actuator 114 and cartridge 106 are reversibly connectable, according to some embodiments.

According to some embodiments, cartridge 106 comprises liquid container 162.

According to some embodiments, liquid container 162 is contained within cartridge internal compartment 108 of cartridge 106.

According to some embodiments, cartridge 106 comprises outlet 110. According to some embodiments, outlet 110 is formed on cartridge housing 102 of cartridge 106.

According to some embodiments, cartridge 106 comprises evaporation heater 120. According to some embodiments, evaporation heater 120 is contained within cartridge internal compartment 108 of cartridge 106.

According to some embodiments, cartridge 106 comprises support 122. According to some embodiments, support 122 is connected to cartridge housing 102 of cartridge 106.

According to some embodiments, cartridge 106 comprises liquid drawing element 164. According to some embodiments, liquid drawing element 164 is connected to cartridge housing 102 of cartridge 106.

According to some embodiments, cartridge 106 further comprises at least one cartridge opening 112 allowing fluid communication between actuator 114 and cartridge 106. Specifically, according to some embodiments, fluid communication between cartridge 106 and actuator 114 may be required because (a) second trigger 150 may be a pressure sensor (e.g. sensor 152); (b) sensor 152 is located in actuator 114; and (c) sensor 152 senses pressure or flow changes correlating with a user inhalation through outlet 110, which is part of cartridge 106.

According to some embodiments, cartridge 106 comprises cartridge power coupling 196. According to some embodiments, cartridge power coupling 196 is contained within cartridge internal compartment 108 of cartridge 106.

According to some embodiments, cartridge 106 comprises evaporation heater electric contact 132 and cartridge electric contacts 134. According to some embodiments, evaporation heater electric contact 132 and cartridge electric contacts 134 are contained within cartridge internal compartment 108 of cartridge 106.

According to some embodiments, cartridge 106 comprises liquid deposition mechanism 160.

According to some embodiments, actuator 114 comprises flow or pressure sensor 152.

According to some embodiments, actuator 114 comprises power source compartment 192.

According to some embodiments, actuator 114 comprises processing unit assembly 173.

According to some embodiments, actuator 114 comprises compartment of processing unit assembly 174.

According to some embodiments, actuator 114 comprises processing unit 190.

According to some embodiments, electronic cigarette 100 further comprises a communication element (not shown) configured to enable wireless communication of electronic cigarette 100 with servers, databases and personal devices (e.g. computers, mobile phones) among others.

According to some embodiments, the communication element provides wireless communication through Bluetooth, WiFi, ZigBee and/or Z-wave.

Reference is now made to FIG. 6A to FIG. 6C. It is to be understood that embodiments referring to FIGS. 6A-6C apply to any electronic cigarettes 100 as presented herein. Specifically, embodiments referring to FIGS. 6A-6C may apply to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and to electronic cigarettes 100 having liquid deposition mechanism 160 as described in FIG. 32.

FIGS. 6A-6C constitute schematic illustrations of actuator 114, according to some embodiments. Actuator 114 comprises an actuator housing 104, a processing unit 190 configured to control operations of the e-cigarette 100, a power source compartment 192 configured to contain, or otherwise house, a power source, such as battery 194, charge socket 186 and actuator power coupling 198.

According to some embodiments, charge socket 186 is in contact with processing unit 190.

According to some embodiments, actuator power coupling 198 is having a proximal surface and a distal surface, wherein actuator power coupling 198 is positioned at the proximal (top) end of actuator housing 104 and is configured to form an electrical contact with cartridge power coupling 196 (shown, for example, in FIG. 14A) when e-cigarette 100 is assembled.

According to some embodiments, actuator power coupling 198 comprises a plurality of actuator power coupling 198ⁿ, such as actuator power coupling 198^{a} and 198^{b} collectively refer to as actuator power coupling 198.

According to some embodiments, actuator power coupling 198 is attached at its distal surface to supporting rib 199.

According to some embodiments, actuator 114 further comprises snap fit fastener 116 configured to snap together processing unit 190 to actuator housing 104, such that processing unit 190 is held in place and remains in place during operation of e-cigarette 100.

According to some embodiments, actuator 114 further comprises inner sleeve 168, a cross section of which is shown for example in FIG. 6C.

According to some embodiments, actuator 114 further comprises a first trigger 140.

According to some embodiments, processing unit 190 comprises at least one central processing unit (CPU). According to some embodiments, processing unit 190 is consisting of CPU.

FIGS. 6B and 6C enable a view of removable cover 142 of actuator 114. According to some embodiments, removable cover 142 is positioned on actuator housing 104. According to some embodiments, removable cover 142 has an open state and a closed state. According to some embodiments, when removable cover 142 is in an open state an access to at least one of charge socket 186 and master switch 187 is enabled. According to some embodiments, when removable cover 142 is in an open state an access charge socket 186 is enabled. According to some embodiments, when removable cover 142 is in an open state an access to master switch 187 is enabled. According to some embodiments, when removable cover 142 is in a closed state an access to at least one of charge socket 186 and master switch 187 is not enabled. Specifically, removable cover 142 is configured to protect charge socket 186 from contamination, such as dust, water and humidity when electronic cigarette 100 is not being charged, according to some embodiments.

Reference is now made to FIGS. 12A, 12B, 14A, 14B, 15, 16A, 16B, 18A, 18B, 19A, 19B, 20, 21A, 21B and 22-24. It is to be understood that some embodiments referring to FIGS. 12, 14A, 14B, 15, 16A, 16B, 18A, 18B, 19A, 19B, 20, 21A, 21B and 22-24 apply to any electronic cigarettes 100 as presented herein. Specifically, a person skilled in the art would appreciate that some embodiments describing these figures are applicable to electronic cigarettes 100 having liquid deposition mechanism 160 as described in FIGS. 1-3, to electronic cigarettes 100 having liquid deposition mechanism 160 as described in FIGS. 4-5, to electronic cigarettes 100 having liquid deposition mechanism 160 as described in FIGS. 28A-C, to electronic cigarettes 100 having liquid deposition mechanism 160 as described in FIGS. 29A-C, to electronic cigarettes 100 having liquid deposition mechanism 160 as described in FIGS. 30A-B, to electronic cigarettes 100 having liquid deposition mechanism 160 as described in FIGS. 31A-B, and/or to electronic cigarettes 100 having liquid deposition mechanism 160 as described in FIG. 32.

FIGS. 12A and 12B constitute schematic illustrations of actuator processing unit assembly 174, according to some embodiments. Processing unit assembly 174 comprises processing unit 190, supporting rib 199, actuator power couplings 198^{a} and 198^{b} attached to supporting rib 199, charge socket 186, board to board connector 188 and sensor 152.

According to some embodiments, sensor 152 is attached, or being in contact with, supporting rib 199.

According to some embodiments, actuator processing unit assembly 174 further comprises master switch 187 configured to turn on/off the operation of e-cigarette 100. According to some embodiments, master switch 187 is an on/off switch, configured to turn on or off processing unit 190. According to some embodiments, master switch 187 is used for power conservation to avoid excessive power consumption by processing unit 190, when electronic cigarette 100 is not operated. According to some embodiments, master switch 187 is accessible from outside of electronic cigarette 100 by a user, as may be shown in FIG. 6C.

According to some embodiments, board to board connector 188 extends between processing unit 190 and supporting rib 199 and being in electrical contact with processing unit 190 and with actuator power couplings 198^{a} and 198^{b}.

According to some embodiments, board to board connector 188 has a distal end in contact with processing unit 190 and a proximal end in contact with supporting rib 199.

According to some embodiments, charge socket 186 comprises a USB charge socket, such as, but not limited to, USB charge socket, mini USB charge socket, micro USB charge socket and USB type C charge socket. According to some embodiments, charge socket 186 is consisting of a USB charge socket. According to some embodiments, charge socket 186 is accessible from outside of electronic cigarette 100 by a user, as may be shown in FIG. 6C. According to some embodiments, charge socket 186 comprises a USB charging port configured for receiving a USB cable (not shown). The USB cable, when connected to the USB charging port, serves as a power charging cable for charging rechargeable battery 194.

According to some embodiments, sensor 152 is a breath sensor. According to some embodiments, sensor 152 is a pressure sensor. According to some embodiments, sensor 152 comprises a plurality of sensors, such as sensor 152^{a} and sensor 152^{b}, wherein at least one sensor is a breath sensor and at least one sensor is a pressure sensor.

According to some embodiments, actuator processing unit assembly 174 further comprises indicator 185, attached to, or in electric contact with, processing unit 190.

According to some embodiments, indicator 185 comprises at least one light source. According to some embodiments, the at least one light source comprises LED. According to some embodiments, the at least one light source is consisting of LED.

According to some embodiments, indicator 185 is configured to indicate the operation of processing unit 190. For example, indicator 185 may provide a green light when processing unit 190 is operated and a red light when processing unit 190 is not operated; or provide light when processing unit 190 is operated and a not light when processing unit 190 is not operated. According to some embodiments, indicator 185 is configured to indicate the operation of electronic cigarette 100. For example, indicator 185 may provide a green light when electronic cigarette 100 is operated and a red light when electronic cigarette 100 is not operated; or provide light when electronic cigarette 100 is operated and a not light when electronic cigarette 100 is not operated.

According to some embodiments, indicator 185 is visible from outside of electronic cigarette 100 by a user.

FIG. 12A constitutes a schematic illustration of actuator processing unit assembly 174, which comprises a piezo inductor 154. According to some embodiments, piezo inductor 154 is controlled by processing unit 190. According to some embodiments, piezo inductor 154 configured to regulate to current induction provided to piezo disc 180. According to some embodiments, piezo inductor 154 is generally beneficial when using liquid deposition mechanism 160 as described when referring to FIGS. 4 and 5.

FIG. 12B constitutes a schematic illustration of actuator processing unit assembly 174, which does not include piezo inductor 154. Specifically, when using liquid deposition mechanisms 160 of the current disclosure, when are configured to provide discrete volumes of liquid intermittently without a piezo mechanism (e.g. liquid deposition mechanism 160 as described in FIGS. 1-3), piezo inductor 154 may not be required.

Reference is now made to FIG. 14A and FIG. 14B. FIGS. 14A and 14B constitute schematic illustrations of a cross-sectional side view of cartridge 106 along the longer axis of e-cigarette 100, according to some embodiments wherein in FIG. 14A cartridge 106 is disassembled from actuator 114, and in FIG. 14B cartridge 106 is connected to actuator 114. Cartridge 106 comprises cartridge housing 102 adapted to aesthetically cover the components of cartridge 106, and also adapted to provide a comfortable grip of cartridge 106 in the context of e-cigarette 100. Cartridge 106 further comprises support 122, evaporation heater 120 placed in an area defined by support 122, wherein evaporation heater 120 comprises evaporation heater electric contacts, such as, cartridge electric contacts 132 and 134. Cartridge 106 further comprises piezo slot 184 adapted to hold there within piezo disc 180 (not shown), and fluid deposition mechanism 160. Fluid deposition mechanism 160 comprises liquid container 162, liquid drawing element 164 being in fluid connection with the liquids in liquid container 162 when the latter if filled with liquid. Cartridge 106 further comprises fluid deposition mechanism housing 178 housing components of liquid deposition mechanism 160, evaporation heater 120 and components functionally and/or structurally related thereto. Cartridge 106 further comprises cartridge compartment 108 housing the components of the liquid deposition mechanism 160, evaporation heater 120, fluid deposition mechanism housing 178 and components structurally associated therewith. Cartridge 106 further comprises cartridge power coupling 196 (e.g. actuator power coupling 196^{a} and 196^{b}) having a proximal surface and a distal surface, wherein cartridge power coupling 196 is attached to the distal (bottom) end of cartridge 106. Cartridge power coupling 196 is configured to form an electrical contact with actuator power coupling 198 (e.g. actuator power coupling 198^{a} and 198^{b}), when e-cigarette 100 is assembled (as shown in counterpart FIG. 14B).

According to some embodiments, cartridge power coupling 196 comprises a plurality of cartridge power coupling 196′, such as cartridge power coupling 196^{a} and 196^{b} collectively refer to as cartridge power coupling 196.

According to some embodiments, cartridge housing 102 comprises outlet 110 adapted to enable delivery of aerosol/vapor formed in cartridge 106 during operation of e-cigarette 100, to the outside, preferably, to the mouth of a user.

According to some embodiments, cartridge 106 further comprises at least one cartridge opening 112. According to some embodiments, cartridge opening 112 is configured to allow passage there through of solenoid plunger head 172 from actuator 114 to cartridge internal compartment 108, as shown, for example, in FIG. 2.

FIGS. 14A and 14B further provide a cross sectional view of liquid drawing element positioning compartment 156. According to some embodiments, and as may be seen in FIGS. 14A and 14B is positioned below liquid drawing element 164.

liquid drawing element positioning compartment 156 is formed within liquid deposition mechanism housing 178. According to some embodiments, 156 comprises a compartment for installing a positioning mechanism (not shown) for proper positioning of liquid drawing element 164 in the longitudinal axis. According to some embodiments, electronic cigarette 100 comprises the positioning mechanism. According to some embodiments, the positioning mechanism is accommodated within liquid drawing element positioning compartment 156.

As detailed above and may be seen in FIGS. 14A and 14B, according to some embodiments, liquid drawing element 164 comprises a top surface in contact with piezo disc 180 and a bottom surface in contact with the positioning mechanism. According to some embodiments, the positioning mechanism is configured to apply pressure on the bottom surface of liquid drawing element 164, such that the top surface of liquid drawing element 164 is pressed against piezo disc 180.

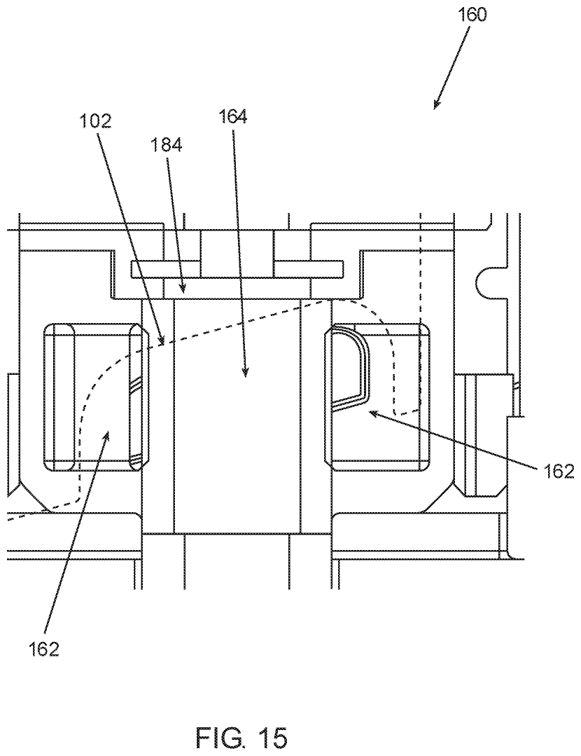

Reference is now made to FIG. 15. FIG. 15 constitute schematic illustrations of fluid deposition mechanism 160 of cartridge 106. Fluid deposition mechanism 160 comprises liquid container 162, liquid drawing element 164, fluid deposition mechanism housing 178 and piezo slot 184. Fluid deposition mechanism 160 is housed within cartridge housing 102 (broken line contour). According to some embodiments, liquid container 162 surrounds liquid drawing element 164, such that the circumference of liquid drawing element 164 is in liquid connection with the liquid within liquid container 162, when the latter is filled with liquid.

According to some embodiments, liquid drawing element 164 is in fluid connection with liquid contained within container 162 (when the latter is filled with liquid).

According to some embodiments, fluid deposition mechanism housing 178 is configured to be in fluid connection with liquid container 162 and with liquid drawing element 164.

According to some embodiments, piezo slot 184 is adapted to hold there within piezo element, such as, piezo disc 180.

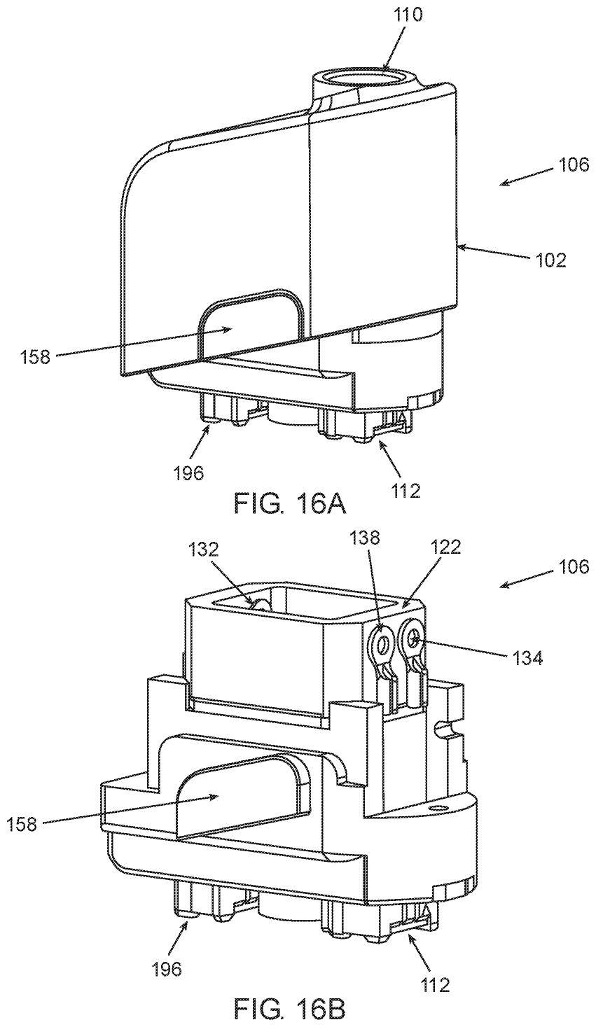

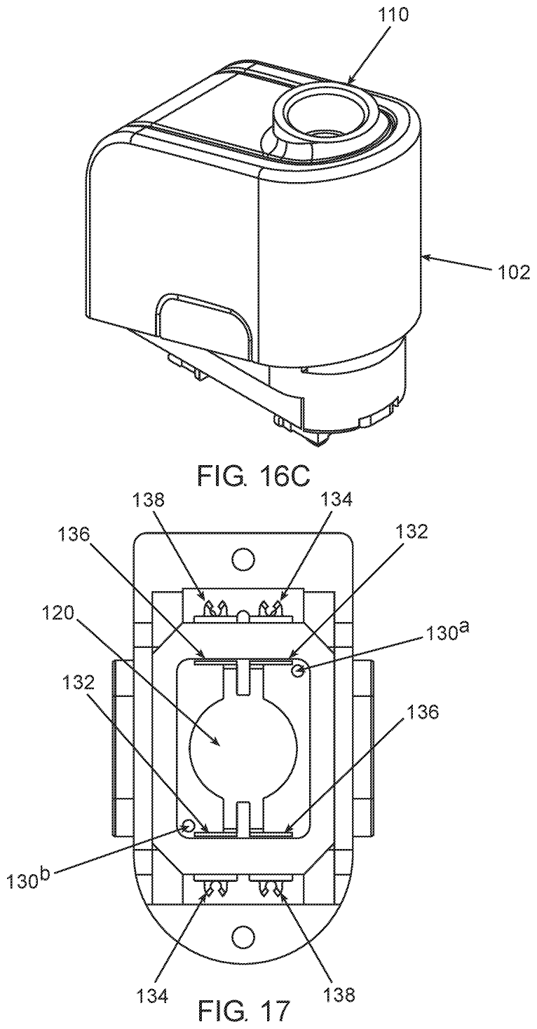

Reference is now made to FIG. 16A, FIG. 16B and FIG. 16C. FIGS. 16A-16C constitute schematic illustrations of cartridge 106, according to some embodiments. FIG. 16A represents side view of cartridge 106, comprising cartridge housing 102, and anchor 158 configured for adjusting the position cartridge housing 102. Cartridge 106 further comprises cartridge power coupling 196. FIG. 16B represents side view of cartridge 106 corresponding to the view shown in FIG. 16A, in the absence of cartridge housing 102 thus exposing some of the components of cartridge 106, such as, evaporation heater electric contacts 132, 134, 138, support 122, cartridge power coupling 196, at least one cartridge opening 112 and anchor 158. FIG. 16C represents side view of cartridge 106 corresponding to the view shown in FIG. 16A, at a lightly different angle providing a better view of outlet 110.

Reference is now made to FIG. 18A and FIG. 18B. FIG. 18A and FIG. 18B constitute schematic illustrations of cross-sectional side views along the longest axis of e-cigarette 100, of cartridge 106, according to some embodiments. Cartridge 106 comprises liquid deposition mechanism 160, anchor 158 and fluid deposition mechanism housing 178 wherein liquid deposition mechanism 160 comprises liquid container 162, piezo slot 184 and fluid deposition mechanism housing 178. Cartridge 106 further comprises support 122 supporting evaporation heater 120 and the evaporation heater electric contacts attached thereto, such as, evaporation heater electric contact 132. According to some embodiments, cartridge 106 further comprises liquid drawing element 164, as shown in FIG. 18B.

FIGS. 18A and 18B further provide a cross sectional view of liquid drawing element positioning compartment 156, the features of which are detailed above. According to some embodiments, liquid deposition mechanism 160 comprises a positioning mechanism accommodated within liquid drawing element positioning compartment 156.

As may be seen in FIGS. 18A and 18B, liquid drawing element positioning compartment 156 may include threads for screwing a bold therein. According to some embodiments, the positioning mechanism comprises a bolt. According to some embodiments, liquid drawing element positioning compartment 156 comprises threads. According to some embodiments, the threads are oriented to allow axial translational of a bolt screwed thereto upwards. According to some embodiments, the threads are oriented to allow axial translational of a bolt screwed thereto from the bottom upwards towards liquid drawing element 164. According to some embodiments, using a bolt as the positioning mechanism provide and adjustable mechanism for positioning liquid drawing element 164.

Reference is now made to FIG. 19A and FIG. 19B. FIG. 19A and FIG. 19B show exemplary configurations of cartridge 106 as shown in FIG. 18B, except that cartridge 106 includes cartridge housing 102 wherein the cross-sectional view shown in FIG. 19A does not present outlet 110, and the cross-sectional view shown in FIG. 19B present outlet 110.

According to some embodiments, outlet 110 is positioned above, and in parallel to, evaporation heater 120. According to alternative embodiments, outlet 110 is positioned above, but not in parallel to, evaporation heater 120, as demonstrated in FIG. 19B.

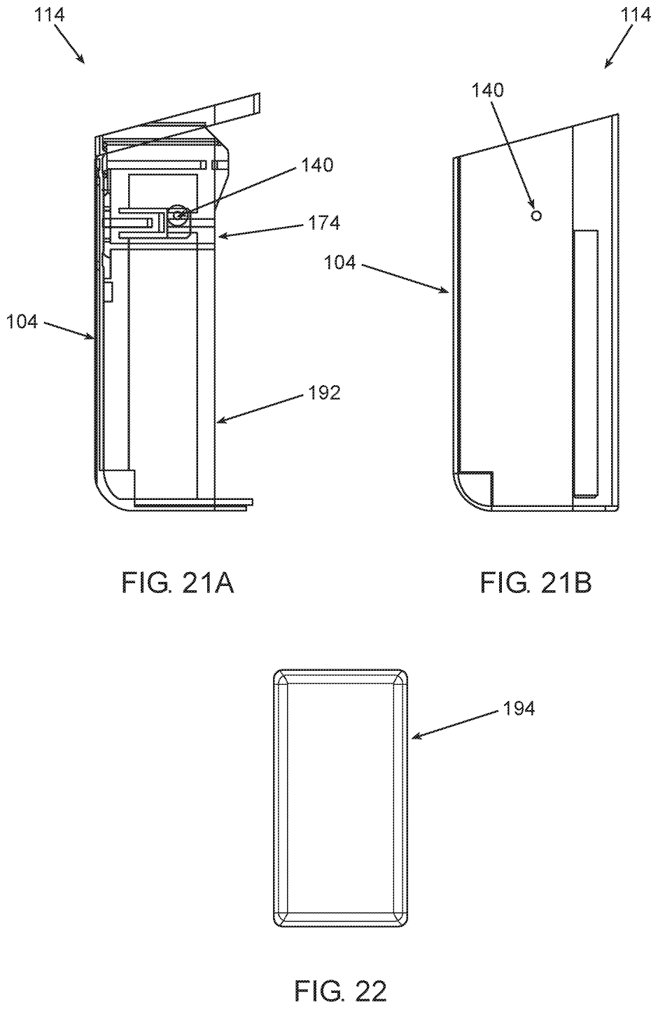

Reference is now made to FIG. 21A and FIG. 21B. FIGS. 21A and 21B constitute schematic illustrations of actuator 114, according to some embodiments. FIG. 21A represents cross-sectional side view of actuator 114, wherein actuator 114 comprises processing unit assembly 174, power source compartment 192, first trigger 140 and actuator housing 104. FIG. 21B represents side view of actuator 114 corresponding to the view shown in FIG. 21A, where actuator housing 104 comprises first trigger 140 and is hiding from view the rest of the components of actuator 114.

Reference is now made to FIG. 22. FIG. 22 constitute schematic illustration of a power source, such as, battery 194 adapted to be housed within a space defined by power source compartment 192.

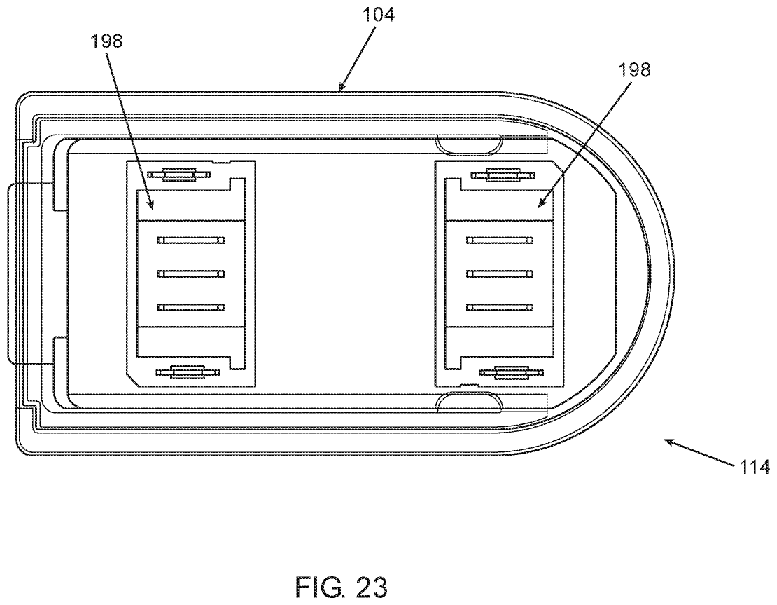

Reference is now made to FIG. 23. FIG. 23 constitute schematic illustrations of a top view of actuator 114, according to some embodiments. Actuator 114 comprises cartridge housing 102, actuator housing 104 enveloping the components of actuator 114 and actuator power coupling 198.

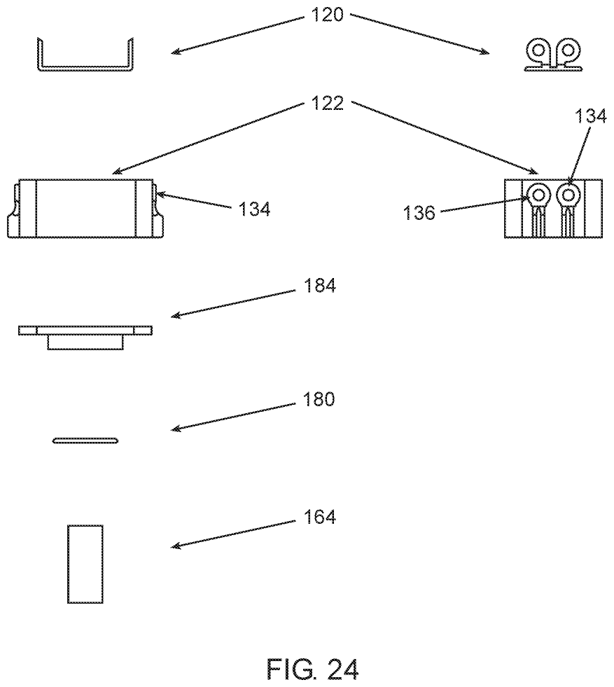

Reference is now made to FIG. 24. FIG. 24 constitute schematic illustrations of selected components of cartridge 106, according to some embodiments. Specifically, FIG. 24 constitute schematic illustrations of first and second side views of evaporation heater 120, at cross-sectional planes perpendicular to one another; first and second side views of cartridge electric contact 134 adjacent, or in contact with support 122, at cross-sectional planes perpendicular to one another; and side views of piezo slot 184, piezo disc 180 and liquid drawing element 164.

Reference is now made to FIGS. 28A-C and 29A-C. FIG. 28A-C constitute a schematic illustration of electronic cigarette 100, according to some embodiments. Electronic cigarette 100 comprises actuator 114 and cartridge 106 configured to detachably attach thereto.

Electronic cigarette 100 further comprises a liquid deposition mechanism 160, having elements located in actuator 114 and in cartridge 106 as detailed below.

Cartridge 106 comprises cartridge internal compartment 108, liquid container 162 and liquid drawing element 164. According to some embodiments, cartridge 106 comprises outlet 110. According to some embodiments, cartridge 106 comprises air inlet 324.

Actuator 114 comprises evaporation heater 120. According to some embodiments, actuator 114 further comprises flow or pressure sensor 152, first trigger 140, processing unit 190 and power source compartment 192. According to some embodiments, actuator 114 further comprises solenoid actuator solenoid actuator 170 and shaft 372.

According to some embodiments, first trigger 140 is a fingerprint sensor.

According to some embodiments, actuator 114 further comprises a niche 128, for introducing cartridge 106.

FIG. 28A constitutes a preliminary phase, when actuator 114 and cartridge 106 are detached. Cartridge 106 is configured to attach to actuator 114 through moving and/or pressing cartridge 106 in the direction of arrow 326. FIGS. 28B-C constitute the first and second phases of electronic cigarette 100, when actuator 114 and cartridge 106 are attached, as described below.

According to some embodiments, evaporation heater 120 comprises a distal surface with high roughness, wherein the degree of roughness forms the high liquid-contact area; or wherein evaporation heater 120 comprises a porous medium, wherein pores of the porous medium forms the high liquid-contact area.

Electronic cigarette 100 is devoid of additional heating elements. Specifically, other than evaporation heater 120, electronic cigarette 100 does not include heaters which are configured to elevate a temperature to an evaporation temperature, according to some embodiments. However, although not presented in a figure, a parallel e-cigarette, having at least one heating element 330 and evaporation medium 320, which replace evaporation heater 120, is contemplated, according to some embodiments.

According to some embodiments, liquid deposition mechanism 160 comprises liquid container 162 and liquid drawing element 164.

Liquid drawing element 164 is partially inserted in liquid container 162, and is configured to absorb liquids therefrom. Liquid drawing element 164 comprises a distal end and a proximal end, wherein the distal end is located inside liquid container 162 and the proximal end extends therefrom, such that the proximal end is not inside liquid container 162. Liquid drawing element 164 is configured to absorb liquids from liquid container 162, through the distal end. According to some embodiments, liquids absorbed to distal end of liquid drawing element 164 flow from the distal end to the proximal end of liquid drawing element 164, such that proximal end of liquid drawing element 164 is absorbed with liquid. According to some embodiments, liquids absorbed to distal end of liquid drawing element 164 flow from the distal end to the proximal end of liquid drawing element 164, such that liquid drawing element 164 is absorbed with liquid.

FIG. 29A constitutes a top view of actuator 114, through niche 128 corresponding to FIG. 28A. FIG. 29B constitutes a top view of actuator 114, through niche 128 corresponding to FIG. 28B. FIG. 29C constitutes a top view of actuator 114, through niche 128 corresponding to FIG. 28C.

According to some embodiments, evaporation heater 120 is connected to main actuator 114, while being out of its frame. According to some embodiments, evaporation heater 120 is connected to actuator 114, while in niche 128 area.

When actuator 114 and cartridge 106 are attached, liquid drawing element 164 is extending from liquid container 162 towards evaporation heater 120 inside niche 128. FIG. 29A constitutes a top view of main housing actuator 114, and shows a top view of evaporation heater 120 when actuator 114 and cartridge 106 are detached. As shown in FIG. 29A, in this preliminary phase, liquid drawing element 164 is not in proximity with evaporation medium 120. FIGS. 29B and 29C constitute top views of actuator 114, and show a top view of evaporation heater 120 when actuator 114 and cartridge 106 are attached. As shown in FIG. 29B, in the first phase of electronic cigarette 100, liquid drawing element 164 is in proximity with evaporation heater 120, but not in contact therewith. As shown in FIG. 29C, in the second phase of electronic cigarette 100, liquid drawing element 164 is in contact with evaporation medium 120.

According to some embodiments, liquid container 162 is configured to contain liquids. According to some embodiments, liquid container 162 contains liquids. According to some embodiments, the liquids are as described above, when referring to electronic cigarette 100 of any one of FIGS. 1-3 and 5. According to some embodiments, the liquid comprises the cannabinoid composition described below.

According to some embodiments, liquid is provided in liquid container 162 for deliverance towards evaporation heater 120 via liquid drawing element 164.

According to some embodiments, liquid drawing element 164 comprises a material that is capable of incorporating, taking in, drawing in or soaking liquids, and upon applying physical pressure thereto or being in contact with another material, release a portion or the entire amount/volume of the absorbed liquid.

According to some embodiments, liquid drawing element 164 is a wick. According to some embodiments, liquid drawing element 164 is configured to absorb liquid in an amount which is at least 100% of its weight. According to some embodiments, liquid drawing element 164 is configured to absorb liquid in an amount which is at least 50% of its weight.

According to some embodiments, liquid drawing element 164 comprises cloth, wool, felt, sponge, foam, cellulose, yarn, microfiber or a combination thereof, having high tendency to absorb aqueous solutions. Each possibility represents a separate embodiment. According to some embodiments, the sponge is an open cell sponge. According to some embodiments, the sponge is a closed cell sponge.

According to some embodiments, liquid drawing element 164 comprises fabric. Specifically, fibrous and/or woven fabric, such as a wick, is a hydrophilic and liquid absorbing material, which may be used as the stationary liquid absorbing element(s), according to some embodiments.

According to some embodiments, liquid drawing element 164 is a hydrophilic liquid drawing element. According to some embodiments, liquid drawing element 164 is a hydrophilic sponge.

Without wishing to be bound by any theory or mechanism of action, when liquid drawing element 164 comprises a hydrophilic sponge, at it comes in contact with the liquid in liquid container 162, capillary action within and among the pores of the sponge lead to it being absorbed.

According to some embodiments, liquid drawing element 164 is in contact with the liquid in liquid container 162. According to some embodiments, liquid drawing element 164 is positioned partially inside liquid container 162, such that it draws liquid therefrom, when liquid container 162 contains liquid. According to some embodiments, liquid drawing element 164 is in placed partially inside liquid container 162, such that it absorbs liquid therefrom, when liquid container 162 contains liquid.

According to some embodiments, liquid deposition mechanism 160 comprises solenoid actuator 170 configured to move evaporation heater 120 towards the liquid drawing element and away therefrom. According to some embodiments, liquid deposition mechanism 160 comprises solenoid actuator 170 configured to move evaporation heater 120 towards the liquid drawing element and away therefrom, when actuator 114 and cartridge 106 are attached. According to some embodiments, liquid deposition mechanism 160 comprises solenoid actuator 170 configured to move evaporation heater 120 towards the liquid drawing element and away therefrom, in the second phase of electronic cigarette 100.

FIGS. 28B and 29B show the first phase of electronic cigarette 100, wherein actuator 114 and cartridge 106 are attached. In this phase, liquid drawing element 164 is in proximity with evaporation heater 120, but is not in contact therewith. As shown in FIGS. 28B and 29B the surfaces of liquid drawing element 164 and evaporation heater 120 are parallel. Upon actuation of solenoid actuator 170, evaporation heater 120 is moved towards liquid drawing element 164, such that there is contact between evaporation heater 120 is moved towards liquid drawing element 164.

The second phase of electronic cigarette 100, wherein evaporation heater 120 is moved towards liquid drawing element 164 to form contact is described in FIGS. 28C and 29C.

Upon the contact, a thin layer of liquid in delivered from liquid drawing element 164, to evaporation heater 120. After the contact has occurred for a pre-determined period of time sufficient for providing evaporation heater 120 with a thin layer of liquid. After the pre-determined period of time solenoid actuator 170 moves evaporation heater 120 to the position shown in FIGS. 28B and 29B. According to some embodiments, the actuation may be repeated a plurality of times. According to some embodiments, upon evaporation of the liquid from evaporation heater 120 solenoid actuator 170 is configured to displace evaporation heater 120, such that it is in further contact with liquid drawing element 164.

The duty cycle and frequency of contact and moving between the first and second phase is detailed above, when describing electronic cigarette 100 of FIGS. 1-3 and 5.

According to some embodiments, liquid deposition mechanism 160 further comprises solenoid actuator 170, configured to move evaporation heater 120 towards liquid drawing element 164 and away therefrom.

According to some embodiments, processing unit 190 is configured to control solenoid actuator 170.

According to some embodiments, processing unit 190 is configured to control solenoid actuator 170, such that upon receiving first trigger activation signal, solenoid actuator 170 moves evaporation heater 120 towards liquid drawing element 164. According to some embodiments, processing unit 190 is configured to control solenoid actuator 170, such that upon receiving first trigger activation signal, solenoid actuator 170 moves evaporation heater 120 towards liquid drawing element 164, such that evaporation heater 120 and liquid drawing element 164 are in contact. According to some embodiments, processing unit 190 is configured to control solenoid actuator 170, such that upon receiving first trigger activation signal, a 170 moves evaporation heater 120 towards liquid drawing element 164; evaporation heater 120 and liquid drawing element 164 are in contact, and a thin layer of liquid is formed on evaporation heater 120. According to some embodiments, processing unit 190 configured to control solenoid actuator 170, such that upon receiving first trigger activation signal, solenoid actuator 170 moves evaporation heater 120 towards liquid drawing element 164 for a predetermined period of time and moves evaporation heater 120 medium away from liquid drawing element 164 after said predetermined period of time, wherein evaporation heater 120 and liquid drawing element 164 are in contact for said predetermined period of time. According to some embodiments, said predetermined period of time is determined such that a thin layer of liquid is formed on evaporation heater 120.

According to some embodiments, Processing unit 190 is configured to control solenoid actuator 170, such that upon receiving first trigger activation signal, solenoid actuator 170 moves evaporation heater 120 towards liquid drawing element 164. According to some embodiments, Processing unit 190 is configured to control solenoid actuator 170, such that upon receiving first trigger activation signal, solenoid actuator 170 moves evaporation heater 120 towards liquid drawing element 164, such that evaporation heater 120 and liquid drawing element 164 are in contact. According to some embodiments, Processing unit 190 is configured to control solenoid actuator 170, such that upon receiving first trigger activation signal, solenoid actuator 170 moves evaporation heater 120 towards liquid drawing element 164; evaporation heater 120 and liquid drawing element 164 are in contact, and a thin layer of liquid is formed on evaporation heater 120. According to some embodiments, Processing unit 190 is configured to control solenoid actuator 170, such that upon receiving first trigger activation signal, solenoid actuator 170 moves evaporation heater 120 towards liquid drawing element 164 for a predetermined period of time and moves evaporation heater 120 away from liquid drawing element 164 after said predetermined period of time, wherein evaporation heater 120 and liquid drawing element 164 are in contact for said predetermined period of time. According to some embodiments, said predetermined period of time is determined such that a thin layer of liquid is formed on evaporation heater 120.

According to some embodiments, solenoid actuator 170 comprises shaft 372, wherein shaft 372 is connected to evaporation heater 120. According to some embodiments, solenoid actuator 170 moving evaporation heater 120 entails solenoid actuator 170 moving shaft 372 thereby moving evaporation heater 120.

Reference is now made to FIGS. 30A-30B. FIGS. 30A-3B constitute a schematic illustration of an electronic cigarette 100, according to some embodiments. Electronic cigarette 100 comprises an actuator 114 and cartridge 106 configured to detachably attach thereto.

According to some embodiments, there is provided an electronic cigarette comprising: an outlet, an evaporation heater comprising a high liquid-contact area, and configured generate heat, such that it is elevated to an evaporation temperature of at least 95° C.; a liquid deposition mechanism comprising a collapsible liquid container, compression spring configured to press the collapsible liquid container, and escapement mechanism configured to block and allow operation of the escapement mechanism and a flap movable upon variation of pressure in the outlet, wherein movement of the flap entails operation of the escapement mechanism; wherein the high liquid-contact area comprises a surface area for contacting liquid being at least one order of magnitude higher than the surface area of a flat non-porous element having the same external dimensions as those of the evaporation heater.

According to some embodiments, there is provided an electronic cigarette 100 comprising: an outlet 110, an evaporation heater 120 comprising a high liquid-contact area, and configured generate heat, such that it is elevated to an evaporation temperature of at least 95° C.; a liquid deposition mechanism 160 comprising a collapsible liquid container 162, compression spring 374 configured to press the collapsible liquid container 162, and escapement mechanism 376 configured to block and allow operation of the escapement mechanism 376 and a flap 177 movable upon variation of pressure in the outlet 110, wherein movement of the flap 177 entails operation of the escapement mechanism 376; wherein the high liquid-contact area comprises a surface area for contacting liquid being at least one order of magnitude higher than the surface area of a flat non-porous element having the same external dimensions as those of the evaporation heater 120.

According to some embodiments, electronic cigarette 100 further comprises a liquid deposition mechanism 160, having elements located in actuator 114 and cartridge 106 as detailed below.

According to some embodiments, cartridge 106 comprises collapsible liquid container 162 and liquid drawing element in the form of a nozzle 164. According to some embodiments, cartridge 106 comprises outlet 110. According to some embodiments, cartridge 106 comprises an air inlet 324.

According to some embodiments, actuator 114 comprises evaporation heater 120. According to some embodiments, actuator 114 further comprises flow or pressure sensor 152, first trigger 140, processing unit 190 and power source compartment 192. According to some embodiments, actuator 114 further comprises an out let in the form of a mouthpiece 110d. According to some embodiments, actuator 114 further comprises compression spring 374 and an escapement mechanism 376. According to some embodiments, actuator 114 further comprises a flap 177.

According to some embodiments, actuator 114 further comprises an electric contact 134, allowing transfer there through of at least one of: electric power supply from power source compartment 192 to evaporation heater 120 and electric signals from processing unit 190 to evaporation heater 120.

According to some embodiments, first trigger 140 is a switch. According to some embodiments, first trigger 140 is a knob. According to some embodiments, first trigger 140 is a dial. According to some embodiments, first trigger 140 is a lever. According to some embodiments, first trigger 140 is a button. According to some embodiments, first trigger 140 is a touch interface.

According to some embodiments, liquid deposition mechanism 160 comprises a collapsible liquid container 162; a compression spring 374 and an escapement mechanism 376.

According to some embodiments, liquid deposition mechanism 160 comprises collapsible liquid container 162; compression spring, 374 escapement mechanism 376 and flap 177. According to some embodiments, liquid deposition mechanism 160 comprises collapsible liquid container 162; compression spring 374 and escapement mechanism 376 comprising a flap 177. According to some embodiments, flap 177 is pressure sensitive and positioned in proximity to outlet or mouthpiece 110 (FIGS. 30A and 30B).

According to some embodiments, liquid deposition mechanism 160 comprises collapsible liquid container 162; compression spring 374 and escapement mechanism 376 comprising flap 177, escapement element 183 and escapement rack 184d. According to some embodiments, flap 177d is functionally connected to escapement element 183d. According to some embodiments, escapement element 183d is configured to control the movement of escapement rack 384. According to some embodiments, escapement rack 384 is configured to control the expansion of compression spring 374. According to some embodiments, the expansion of compression spring 374 entails reducing the volume of collapsible liquid container 162.

FIG. 30A constitutes a phase when flap 177 is not experiencing differential pressure between both sides thereof. As a result, and as detailed below, escapement rack 384 is blocked and restrains compression spring 374 from expansion, such that collapsible liquid container 162 is not compressed to exert liquid through nozzle 164.

FIG. 30B constitutes a phase when flap 177 is experiencing differential pressure between both sides thereof, as a result of an inhalation through mouthpiece 110. As a result, and as detailed below, escapement rack 384 is released and allows expansion of compression spring 374, such that collapsible liquid container 162 is compressed to exert liquid through nozzle 164.

According to some embodiments, reducing the volume of collapsible liquid container 162 entails flow of liquid contained therein from collapsible liquid container 162 to evaporation heater 120 through a nozzle 164 extending from collapsible liquid container 162 to evaporation heater 120. According to some embodiments, said flow of liquid is in an amount to form a thin layer of the liquid on evaporation heater 120. According to some embodiments, reducing the volume of collapsible liquid container 162 entails flow of liquid contained therein from collapsible liquid container 162 to the evaporation heater through nozzle 164 extending from collapsible liquid container 162 to the evaporation heater. According to some embodiments, said flow of liquid is in an amount to form a thin layer of the liquid on evaporation heater 120. According to some embodiments, liquid deposition mechanism 160 is designed such that reduced pressure experienced by flap 177 (e.g. due to inhalation through mouthpiece 110) results in reducing the volume of collapsible liquid container 162.

According to some embodiments, collapsible liquid container 162 comprises nozzle 164 having an orifice (not shown) located in close proximity with evaporation heater 120. According to some embodiments, liquid deposition mechanism 160 comprises a nozzle fluidly connected to collapsible liquid container 162.

According to some embodiments, collapsible liquid container 162 comprises nozzle 164 having an orifice located in close proximity with evaporation heater 120.

According to some embodiments, compression spring 374 comprises a proximal end and a distal end, wherein the distal end is mounted to spring base 175 facing mouthpiece 110 and the proximal end is mounted to the support 386, wherein support 386 is connected to evaporation heater 120, and in fluid contact with collapsible liquid container 162. According to some embodiments, compression spring 374 comprises a proximal end and a distal end, wherein the distal end is mounted to spring base 175 facing 110 and the proximal end is mounted to support 386, wherein support 386 is connected to evaporation heater 120, and in fluid contact with collapsible liquid container 162, such that upon expansion of the spring 374, evaporation heater 120 is moved away from mouthpiece 110. According to some embodiments, compression spring 374 comprises a proximal end and a distal end, wherein the distal end is mounted to spring base 175 facing mouthpiece 110 and the proximal end is mounted to support 386, wherein support 386 is connected to evaporation heater 120, and in fluid contact with collapsible liquid container 162, such that upon expansion of spring 374, collapsible liquid container 162 is squeezed, thereby reducing in volume and delivering liquid contained therein through nozzle 164 and orifice to evaporation heater 120.

According to some embodiments, compression spring 374 comprises a proximal end and a distal end, wherein the distal end is mounted to spring base 175 facing mouthpiece 110 and the proximal end is mounted to support 386, wherein support 386 is connected to the evaporation heater 120, and in fluid contact with collapsible liquid container 162. According to some embodiments, compression spring 374 comprises a proximal end and a distal end, wherein the distal end is mounted to spring base 175 facing mouthpiece 110 and the proximal end is mounted to support 386, wherein support 386 is connected to evaporation heater 120, and in fluid contact with collapsible liquid container 162, such that upon expansion of spring 174, evaporation heater 120 is moved away from mouthpiece 110. According to some embodiments, compression spring 374 comprises a proximal end and a distal end, wherein the distal end is mounted to spring base 175 facing mouthpiece 110 and the proximal end is mounted to support 386, wherein support 386 is connected to evaporation heater 120, and in fluid contact with collapsible liquid container 162, such that upon expansion of the spring 374, collapsible liquid container 162 is squeezed, thereby reducing in volume and delivering liquid contained therein through nozzle 164 and its orifice to evaporation heater 120.

According to some embodiments, escapement mechanism 376 is configured to restrain compression spring 374 from expanding. According to some embodiments, escapement mechanism 376 is further configured to allow compression spring 374 to expand.

According to some embodiments, escapement mechanism 376 comprises flap 177 movable about axis 378 and located in proximity with mouthpiece 110. According to some embodiments, flap 177 is elongated and has a first and second ends, wherein the flap 177 movable about axis 378 in the first end, and free in the opposite second end.

According to some embodiments, flap 177 and axis 378 are located such that when in atmospheric pressure flap 177 is substantially parallel to evaporation heater 120 (see FIG. 30A) and upon application of reduced pressure on mouthpiece 110 (e.g. by inhalation) flap 177 is drawn to be vertical or diagonal to evaporation heater 120 (FIG. 30B). According to some embodiments, flap 177 is movable about an axis 378 located in proximity with mouthpiece 110. According to some embodiments, flap 177 and axis 378 are located such that when in atmospheric pressure flap 177 is parallel to evaporation heater 120 (FIG. 30A); and upon application of reduced pressure on mouthpiece 110 (e.g. by inhalation) flap 177 is drawn to be vertical or diagonal to evaporation heater 120 (FIG. 30B). According to some embodiments, upon flap 177 moving to be vertical to evaporation heater 120 (FIG. 30B), the second end moves towards mouthpiece 110. According to some embodiments, flap 177 comprises an inner position 179 located between the first and second end. According to some embodiments, upon flap 177 moving to be vertical to evaporation heater 120 (FIG. 30B), inner position 179 moves towards mouthpiece 110.

According to some embodiments, escapement mechanism 376 comprises drawbar 380 having a first end connected to inner position 179 of flap 177 and a second end connected to a shaft 181 through an axis 382. According to some embodiments, upon application of reduced pressure and moving of inner position 179 towards mouthpiece 110 (FIG. 30B), drawbar 380 is also moved towards mouthpiece 110.

According to some embodiments, shaft 181 comprises a first end connected to drawbar 380 though axis 382 and a second side rigidly connected to an escapement element 183, which is vertical thereto.

According to some embodiments, escapement element 183 is located over escapement rack 384 having a plurality of teeth 189, such that when escapement element 183 is aligned parallel to escapement rack 384 (FIG. 30B), escapement rack 384 is movable, and when escapement element 183 is aligned diagonally to escapement rack 384 (FIG. 30A), escapement element 183 located between two of plurality of teeth 189, thereby blocking the movement of escapement rack 384.

According to some embodiments, upon application of reduced pressure and moving of drawbar 380 towards mouthpiece 110, escapement element 183 is rotated from parallel alignment to diagonal alignment with respect to escapement rack 384.

According to some embodiments, escapement rack 384 is connected to support 386. According to some embodiments, when escapement rack 384 is movable (FIG. 30B), support 386 may be moved by compression spring 374, and when escapement rack 384 is blocked (FIG. 30A), support 386 is fixed, such that compression spring 374 is restrained.

According to some embodiments, liquid deposition mechanism 160 comprises collapsible liquid container 162, escapement mechanism 376 and compression spring 374 having a pressure sensitive flap 177, such that upon inhalation flap 177 operates escapement mechanism 376 to allow compression spring 374 to expand and squeeze collapsible liquid container 162, such that it spreads a thin layer of liquid over evaporation heater 120 (FIG. 30B).

It will be clear that the embodiments described and illustrated in conjunction with FIGS. 30A-B relate to an electronic cigarette 100 provided with a liquid deposition mechanism 160 that does not transition between two state, the first of which includes a liquid deposition mechanism 160 is spaced apart from evaporation heater 120, but rather a liquid deposition mechanism 160 is always in contact with the evaporation heater 120, but may transition between a state in which liquid is not deposited onto evaporation heater 120 (see FIG. 30A), and a state in which liquid deposition mechanism 160 is delivering a discrete volume of liquid onto evaporation heater 120, and the discrete volume of liquid is evaporated and subsequently aerosolized, due to evaporation heater 120 being in an elevated evaporation temperature (see FIG. 30B).

Reference is now made to FIG. 31A-31B. FIGS. 31A and 31B constitute partial views of electronic cigarette 100 during the first and second state, respectively.

FIG. 31A shows electronic cigarette 100 in a first state. According to some embodiments, first trigger 140 is a switch. According to some embodiments, first trigger 140 is a knob. According to some embodiments, first trigger 140 is a dial. According to some embodiments, first trigger 140 is a lever. According to some embodiments, first trigger 140 is a button. According to some embodiments, first trigger 140 is a touch interface.

During the first state, the user activates the first trigger 140, for example by pushing a switch-type first trigger 140 with his/her finger. first trigger 140 is configured to at least trigger activation or deactivation of at least one heating element 330, according to some embodiments. As a result of the user pushing switch 140, first trigger 140 is activated. In addition, a user is drawing from electronic cigarette 100 by inserting outlet or mouthpiece 110 into his/her mouth and drawing an inhalation. As a result of the drawing a pressure decrease is felt inside the confines of housing 102 and a flow of air commences (shown in FIG. 31A as broken-line arrow). As detailed above, according to some embodiments, flow sensor or a pressure sensor 152 is configured to detect flow or the pressure, respectively. As a result, flow sensor or a pressure sensor 152 detects the flow or the pressure, which results from the drawing, and activates second trigger 150.

As detailed above, according to some embodiments, processing unit 190 is configured to receive signals from both first trigger 140 and from second trigger 150 and is further configured to control operation at least one heating element 330. Thus, the result of the user pushing switch 140 and inhaling from outlet 110 is the operation of least one heating element 330. According to some embodiments, at least one heating element 330 is configured to rapidly transfer heat to evaporation medium 320. Specifically, according to some embodiments, at least one heating element 330 is configured to transfer sufficient heat to elevate the temperature of evaporation medium 320 to evaporation temperature.

According to some embodiments, evaporation medium 320 is having a distal surface, which is in contact with temperature sensor 131. According to some embodiments, temperature sensor 131 is configured to detect the temperature of evaporation medium 320 and to send a temperature signal, indicative of said temperature to processing unit 190. Thus, upon elevation of the temperature of evaporation medium 320 to evaporation temperature, processing unit 190 receives a signal indicative of the temperature and controls the current delivered to at least one heating element 330, such that overheating of evaporation medium 320 is avoided.

FIG. 31B show the second state. As detailed above, both first trigger 140 and first trigger 150 are configured to trigger activation or deactivation of liquid deposition mechanism 160. As further detailed above, processing unit 190 is configured to control operation liquid deposition mechanism 160. The drawing from outlet 110 and pushing of switch 140 results in the activation of liquid deposition mechanism 160. Liquid deposition mechanism 160 is configured to provide liquid from liquid container 162 to evaporation medium 320. As shown in FIG. 31B, processing unit 190 operates liquid deposition mechanism 160, such that it approaches evaporation medium 320, such that liquid deposition mechanism 160 is in close proximity or in contact with evaporation medium 320. As a result, during the second state a film of liquid contained in liquid container 162 is provided to evaporation medium 320, which is present at evaporation temperature, after the drawing from outlet 110 and pushing of switch 140. As evaporation medium 320 is both wet with the liquid and present at evaporation temperature, evaporation may initiate.

In addition to the initiation of evaporation, the temperature of evaporation medium 320 begins to decline as a result of the contact with the cold liquid (which is maintained at ambient temperature prior to operation). Upon decline of the temperature of evaporation medium 320, processing unit 190 receives a signal indicative of the decreased temperature and, if it declines close to evaporation temperature, processing unit 190 controls the current delivered to at least one heating element 330, such that heating evaporation medium 320 is amplified.

Thereafter, the liquid form liquid deposition mechanism 160 may be exhausted. In such case, the heat energy formed in heating element 330 and transferred to evaporation medium 320 is beginning to be absorbed in evaporation medium 320, thereby raising it temperature. Upon possible elevation of the temperature of evaporation medium 320 to the elevated temperature, processing unit 190 receives a signal indicative of the temperature from temperature sensor 131 and controls the current delivered to at least one heating element 330, such that overheating of evaporation medium 320 is avoided.

A subsequent step of operation may be similar in its configuration to the first state illustrated in FIG. 31A. Specifically, as above, processing unit 190 is configured to control operation of liquid deposition mechanism 160. After operating liquid deposition mechanism 160 to move towards evaporation medium 320, processing unit 190 operates to move to its previous position. As detailed when referring to the second state, evaporation medium 320 is ready for initiation of vaporization of the liquid dispersed thereon of the beginning of this subsequent step. Thus, upon returning of liquid deposition mechanism 160 to the position shown in FIG. 31A, flow of evaporated liquid flows, now present as gaseous evaporated composition towards outlet 110 (shown in FIG. 31A as broken-line arrow). During the course of flow of gaseous evaporated composition towards outlet 110, it experiences lower temperature than the evaporation temperature experienced adjacent to evaporation medium 320. As a result of the decreased temperature, some of the gaseous evaporated composition is condensed to small droplets. Said droplets act as nucleation sites for the condensation of the remaining gaseous evaporated composition, such that an aerosol is generated. The aerosol proceeds in the flow direction through outlet 110, to the mouth of the user.

A yet further subsequent step, similar in its configuration to the configuration of FIG. 31A, is optional, and exhibits a configuration in which the electronic cigarette 100 either commences or terminates the evaporation. Specifically, in scenario (i) the user stops drawing the aerosol, resulting in the pressure inside electronic cigarette 100 reaching approximately atmospheric pressure.

Similar to the first state described above, flow sensor or a pressure sensor 152 detects the flow or the pressure, which results from the drawing termination, and activates second trigger 150. Processing unit 190 is configured to receive termination signals from second trigger 150 and is further configured to terminate the operation of at least one heating element 330. Thus, the result of the user terminating the inhalation is the termination of operation of least one heating element 330, resulting in a temperature decrease.

In scenario (ii) the user continues drawing the aerosol, resulting in the pressure inside electronic cigarette 100 being sub-atmospheric pressure. Similar to the first state described above, flow sensor or a pressure sensor 152 detects the flow or the pressure, which results from the drawing commencement, and activates second trigger 150. Processing unit 190 is configured to receive signals from second trigger 150 and is further configured to continue the operation at least one heating element 330. Thus, the result of the user continuing the inhalation is the continuation of operation of least one heating element 330, resulting in an evaporation temperature, and provision of additional aerosol, as described when referring to the subsequent step after the second state above.

Reference is now made to FIG. 32A. FIG. 32A constitutes a schematic illustration of an electronic cigarette 100, according to some embodiments. According to some embodiments, electronic cigarette 100 comprises a housing 102 and cartridge 106 configured to detachably attach thereto. Cartridge 106 comprises cartridge internal compartment 108 and outlet 110.

Cartridge internal compartment 108 comprises evaporation medium 320, at least one heating element 330 and a portion of liquid deposition mechanism 160. According to some embodiments, cartridge internal compartment 108 further comprises support 122 attached to evaporation medium 320. According to some embodiments, liquid deposition mechanism 160 is configured to provide a thin film of liquid having thickness and/or volume as described above with respect to liquid deposition mechanism 160.

Housing 102 comprises first trigger 140 in the form of a proximity sensor, second trigger 150 in the form of a button, processing unit 190, power source compartment 192 and the remainder portion of liquid deposition mechanism 160.

According to some embodiments, cartridge 106 and housing 102 further comprises evaporation heater electric contact 132 and cartridge electric contact 134, respectively, configured to contact each other to close an electrical circuit between cartridge 106 and housing 102.

According to some embodiments, liquid deposition mechanism 160 comprises a plurality of liquid drawing elements.

Liquid deposition mechanism 160 comprises liquid container 162 and two liquid drawing elements: liquid drawing element in the form of a stationary wick 364, and liquid drawing element in the form of a mobile wick 365, movable by a rack 478 attached thereto. Stationary wick 364 is immobile. Stationary wick 364 is fluidly connected to liquid container 162. According to some embodiments, stationary wick 364 is in contact with the liquid in liquid container 162, configured to absorb a portion of liquid therefrom.

Mobile wick 365 is movable between an absorption position in the first state, and a wetting position in the second state. Absorption position is defined when mobile wick 365 is at least in partial contact with stationary wick 364 (see FIG. 32A). Wetting position is defined when mobile wick 365 is at least in partial contact with evaporation medium 320 (not shown). When mobile wick 365 is in the absorption position, it absorbs a portion of liquid from stationary wick 364, ready to provide a portion of the absorbed liquid to evaporation medium 320 when moved to the wetting position, or to the second state.

Liquid deposition mechanism 160 further comprises a gear motor 368 and a gear 476 driven thereby, configured to fit with rack 478 moving it upon activation of liquid deposition mechanism 160 between an absorption position and a wetting position (or between the first and second states).

According to some embodiments, gear motor 368 is configured to intermittently move gear 476, such that rack 478 intermittently moves mobile wick 365 between the absorption position and the wetting position (i.e., between the first state and the second state).

According to some embodiments, gear motor 368 is configured to intermittently move gear 476, such that rack 478 intermittently moves mobile wick 365 between the absorption position and the wetting position; allowing mobile wick 365 to spread a thin layer of the liquid along evaporation medium 320.

According to some embodiments, spreading of a thin layer of liquid along evaporation medium 320 from mobile wick 365 may be achieved through application of appropriate pressure, or by delicate contact between the two elements.

According to some embodiments, spreading of a thin layer of liquid along evaporation medium 320 from mobile wick 365 is achieved through maintaining contact between the two elements for an appropriate period of time.

According to some embodiments, spreading of a thin layer of liquid along evaporation medium 320 from mobile wick 365 is achieved through fabrication of mobile wick 365 from an appropriate material or in an appropriate manner.

According to some embodiments, processing unit 190 is configured to operate gear motor 368, such that the moving of rack 478 and mobile wick 365 between the absorption and wetting positions is repeated for a plurality of cycles. According to some embodiments, processing unit 190 is configured to intermittently operate gear motor 368, such that the moving of rack 478 and mobile wick 365 between the absorption and wetting positions is repeated for a plurality of cycles.

According to some embodiments, processing unit 190 is configured to intermittently operate gear motor 368, such that the moving of rack 478 and mobile wick 365 between the absorption and wetting positions is repeat for a plurality of cycles, such that the deliveries of the liquid from mobile wick 365 to evaporation medium 320 are not continuous.

According to some embodiments, liquid deposition mechanism 160 is further configured to be deactivated upon delivery of the liquid from mobile wick 365 to evaporation medium 320.

According to some embodiments, mobile wick 365 is deactivated when in the absorption position and activated in the wetting position.

According to some embodiments, liquid deposition mechanism 160 is further configured to return upon deactivation thereof, via the driving unit, back to the first state.

According to some embodiments, the deactivation is scheduled by processing unit 190 immediately after the spreading of liquid by liquid deposition mechanism 160. According to some embodiments, processing unit 190 is configured to intermittently operate gear motor 368, such that the moving of rack 478 and mobile wick 365 between the absorption and wetting positions results in spreading of the thin layer of the liquid along evaporation medium 320.

According to some embodiments, the activation of liquid deposition mechanism 160 entails moving of mobile wick 365 to the wetting position via gear motor 368, gear 476 and rack 478; remaining in the wetting position for a predetermined period of time; and returning of mobile wick 365 to the absorption position; wherein the predetermined period of time is sufficient of delivery of a thin layer of the liquid to evaporation medium 320.

According to some embodiments, mobile wick 265 is fabricated such that moving of mobile wick 265 to the wetting position via gear motor 368, gear 476 and rack 478 and its remaining in the wetting position for a predetermined period of time results in the delivery of a thin layer of liquid to evaporation medium 320.

According to some embodiments, cartridge internal compartment 108 comprises liquid container 162, stationary wick 364, mobile wick 365 and rack 478, while housing 102 comprises gear motor 368 and gear 476. Cartridge opening 112 is configured to allow passage there through of at least one of: a portion of rack 478 (not shown) and a portion of gear 476 (see FIG. 32A).

According to some embodiments, processing unit 190 is configured to operate liquid deposition mechanism 160 in a reciprocating mode while liquid deposition mechanism 160 is activated, such that gear motor 368 reciprocally rotates gear 476 to move rack 478 between an absorption position and a wetting position, so as to enable non-continuous wetting of evaporation medium 320.

Reference is now made to FIG. 32B. FIG. 32B constitutes a schematic illustration of an electronic cigarette 100, according to some embodiments. The electronic cigarette 100 illustrated in FIG. 32B is similar to that of FIG. 32A in function and structure, except that the at least one heating element 330 and evaporation medium 320 from FIG. 32A are replaced by a single unified evaporation heater 320. Electronic cigarette 100 is devoid of additional heating elements. Specifically, other than evaporation heater 320 electronic cigarette 100 does not include heaters which are configured to elevate a temperature to an evaporation temperature.

Provided herein are cannabinoid compositions for the administration of a cannabinoid via inhalation, according to some embodiments. Advantageously, the cannabinoid compositions disclosed herein are provided in an aqueous medium, such as an aqueous medium, which is substantially devoid of organic solvents, according to some embodiments. In addition, the THC compositions disclosed herein, when delivered via inhalation, exhibit THC delivery properties similar to cigarettes, thereby providing an efficient substitute for smokers, which is safer and is devoid of smoke harmful organic decomposition contaminants. According to some embodiments, the cannabinoid composition comprises an aqueous solution comprising at least one cannabinoid compound, such as a cannabinoid acid or a salt thereof. According to some embodiments, the aqueous solution has a pH of at least 9. According to some embodiments, the cannabinoid composition consists of an aqueous solution comprising at least one cannabinoic acid or salt thereof. According to some embodiments, the aqueous solution has a pH of at least 9.

The present invention provides aqueous solutions of cannabinoids which are useful for inhalation by a subject. These aqueous solutions are achieved by forming salts of the cannabinoid acids found in the cannabis plant by contact with an aqueous base, at a pH of 9 or higher. Advantageously the cannabinoid salts so formed are stable in the aqueous solution. Furthermore, the cannabinoid salts can be generated without extraction of the cannabinoids by use of organic solvents or organic co-solvents.

The term “solution” as used herein broadly refers to a combination, mixture and/or admixture of ingredients having at least one liquid component. Thus, the term “aqueous solution” refers to any solution, in which at least one of its liquid components is water, wherein at least 50% of its weight is water. Aqueous solutions typically include water in greater quantity or volume than a solute. Typical additional solvents include alcohols, aldehydes, ketones, sulfoxides, sulfones, nitriles and/or any other suitable solubilizing molecule or carrier compound. Preferably, “solution” refers broadly to a mixture of miscible substances, where one substance dissolves in a second substance. More preferably, in a solution the essential components are homogeneously mixed and that the components are subdivided to such an extent that there is no appearance of light scattering visible to the naked eye when a one inch diameter bottle of the mixture is viewed in sunlight.

According to some embodiments, there is provided a cannabinoid composition for use in the administration of a cannabinoid via inhalation, the composition comprises an aqueous solution comprising at least one cannabinoic acid or salt thereof, wherein the aqueous solution has a pH of at least 9. According to some embodiments, there is provided a cannabinoid composition, the composition comprises an aqueous solution comprising at least one cannabinoic acid or salt thereof, wherein the aqueous solution has a pH of at least 9. According to some embodiments, the cannabinoid composition is consisting of the aqueous solution.

The term “cannabinoid”, as used herein, includes all major and minor cannnabinoids found in natural cannabis and hemp material that can be isolated from a natural source or reproduced by synthetic means. This includes delta-9-Tetrahydrocannabinol (THC), delta-9-Tetrahydrocannabinolic acid (THCA), delta-8-Tetrahydrocannabinol, Cannabidiol (CBD), Cannabidiolic acid (CBDA), Cannabinol (CBN), Cannabinolic acid (CBNA), tetrahydrocannabinovarin (THCV), cannabidivarin (CBDV), cannabigerol (CBG), cannabigerolic acid (CBGA) and cannabichromene (CBC). The term “cannabinoid” also includes basic salts of the acid mentioned above, for example, THCA-sodium salt and THCA-potassium salt.

The term “tetrahydrocannabinolic acid” and “THAC acid” are interchangeable and refer to common derivatives of THC, which are substituted in position 2 of the aromatic ring by a carboxylic acid. THC has two dominant isomers, Δ⁹-THC and Δ⁸-THC. Accordingly, THCA has corresponding Δ⁹ and Δ⁸ isomers. It is to be understood that although the natural THC isomers include an n-C₅H₁₁ chain in position 3, derivatives of THC may include other substituents. Therefore, the term tetrahydrocannabinolic acid includes corresponding structures, in which position 3 is substituted by a group, which is either an n-C₅H₁₁ or a different chemical group. The term “tetrahydrocannabinolic acid” should be interpreted broadly referring to all possible stereoconfigurations and salts of the relevant formula. As used herein the terms “formulation” and “compositions” generally refer to any mixture, solution, suspension or the like that contains an active ingredient, such as cannabinoid, and, optionally, a carrier. The carrier may be any carrier acceptable for smoking, that is compatible for delivery with the active agent.

According to some embodiments, the administration of the cannabinoid via inhalation comprises generating an inhalable aerosol of the cannabinoid composition. According to some embodiments, the administration of the cannabinoid via inhalation comprises generating an inhalable aerosol of the cannabinoid composition upon heating the cannabinoid composition in an aerosol generating device. According to some embodiments, the aerosol generating device is the electronic cigarette disclosed herein.

Surprisingly, it was found that the basic (pH of at least 9, or at least 10) is suitable for delivery to e-cigarette user. Specifically, although such basic compositions are not suitable for direct use of human, it was found that upon aerosolization, a substantially pH neutral aerosol formed, which is compatible with inhalation. Without wishing to be bound by any theory of mechanism of action, the basic cannabinoid composition comprises non-volatile bases, which are not aerosolized, and organic material, comprising THCA, present mainly as a basic salt, e.g. THCA-sodium salt. Upon heating and aerosolization with the electronic cigarette, THCA-sodium salt, which is in equilibrium with THCA, undergoes decarboxylation to form THC, which is aerosolized together with the water medium. THC is pH neutral, therefore, the aerosol is substantially neutral and suitable for the use of human subjects.

According to some embodiments, there is provided a cannabinoid composition for use in the administration of a cannabinoid to a user via inhalation for the treatment of a disease, disorder or symptom, the composition comprises an aqueous solution comprising at least one cannabinoic acid or salt thereof, wherein the aqueous solution has a pH of at least 9. According to some embodiments, the use is for the treatment of a disease, disorder or symptom amenable to treatment with THC. According to some embodiments, the disease, disorder or symptom amenable to treatment with THC is selected from the group consisting of pain, impaired neurological function, inflammation, nausea, vomiting, convulsions, low appetite and glaucoma.

It is to be understood to a person skilled in the art that THCA is an organic acid, and thus is better soluble in water, when the pH is elevated. Specifically, at higher (more basic) pH organic acids are present as salts, which are typically more water soluble then their corresponding acids.

According to some embodiments, the aqueous solution has a pH of at least 9.5. According to some embodiments, the aqueous solution has a pH of at least 10. According to some embodiments, the aqueous solution has a pH of at least 10.5. According to some embodiments, aqueous solution has a pH in the range of 9.5 to 11.5. According to some embodiments, aqueous solution has a pH in the range of 9 to 11. According to some embodiments, aqueous solution has a pH in the range of 10 to 11. According to some embodiments, aqueous solution has a pH in the range of 10.5 to 11.5.

According to some embodiments, the concentration of the at least one cannabinoic acid or salt thereof in the aqueous solution is in the range of 2% to 10% w/w. According to some embodiments, the concentration of the at least one cannabinoic acid or salt thereof in the aqueous solution is in the range of 4% to 6% w/w. According to some embodiments, the percentage of the at least one cannabinoic acid or salt thereof in the cannabinoid compositions is about 5% w/w.

As used herein, the term “about” refers to a range of values ±20%, or ±10% of a specified value. For example, the phrase “the percentage is about 5% w/w” includes ±20% of 5, or from 4% to 6%, or from 4.5% to 5.5%.

As used herein, when relating to cannabinoid percentages in liquid compositions, unless specified otherwise, the volume ratio, or w/w % is referred. For example, the phrase “the percentage of the at least one cannabinoic acid or salt thereof is within the range of 4 to 6%” refers to a liquid solution, in which a single weight unit of the solution includes from 0.04 to 0.06 the weight unit of cannabinoic acid or salt thereof. Specifically, adding 5 gr of THCA to 95 gr of water will result in a 100 gr solution of 5% THCA.

According to some embodiments, cannabinoid composition is a pharmaceutical composition. According to some embodiments, the cannabinoid composition may comprise one or more active agents, other than cannabinoid(s). According to some embodiments, the one or more active agents include one or more pharmaceutically active agents. According to some embodiments, the one or more active agents are suitable or may be adjusted for inhalation. According to some embodiments, the one or more pharmaceutically active agents are directed for treatment of a medical condition through inhalation. According to some embodiments, the medical condition is amenable to treatment with THC. According to some embodiments, the medical condition is selected from the group consisting of pain, impaired neurological function, inflammation, nausea, vomiting, convulsions, low appetite and glaucoma.

According to some embodiments, the cannabinoid composition further comprises an additive selected from the group consisting of a carrier, a preservative, an anti-coughing agent, a stabilizer, a propellant and a flavorant. Each possibility represents a separate embodiment.

According to some embodiments, the additive is an additive acceptable for inhalation. According to some embodiments, the additive is approved for use in inhaling solutions. According to some embodiments, the additive is stable under basic pH conditions. According to some embodiments, the additive is water soluble under basic pH conditions. According to some embodiments, the pharmaceutical composition further comprises at least one pharmaceutically acceptable additive, which is acceptable for inhalation. According to some embodiments, the pharmaceutically acceptable additive is stable under basic pH conditions. According to some embodiments, the pharmaceutically acceptable additive is water soluble under basic pH conditions.

According to some embodiments, the concentration of the at least one additive is in the range of 0.1-1% w/w on the cannabinoid composition. According to some embodiments, the concentration of the at least one additive is in the range of 0.1-0.5% w/w on the cannabinoid composition. According to some embodiments, the concentration of the at least one additive is in the range of 0.1-0.3% w/w on the cannabinoid composition.

According to some embodiments, the flavorant is a sweetener. According to some embodiments, the sweetener is selected from the group of artificial sweeteners including saccharine, aspartame, dextrose and fructose.

According to some embodiments, the additive is selected from menthol, eucalyptol, tyloxapol and a combination thereof. According to some embodiments, the additive is selected from menthol, eucalyptol, tyloxapol and a combination thereof, and is present at a concentration of 0.1-0.5% w/w based on the total weight of the cannabinoid composition.

According to some embodiments, the preservative is selected from the group consisting of benzyl alcohol, propylparaben, methylparaben, benzalkonium chloride, phenylethyl alcohol, chlorobutanol, potassium sorbate, phenol, m-cresol, o-cresol, p-cresol, chlorocresol and combinations thereof.

The term “anti-coughing agent” as used herein refers to an active agent used for the suppression, alleviation or prevention of coughing and irritations and other inconveniencies in the large breathing passages that can, or may, generate coughing. Anti-coughing agent include, but are not limited to antitussives, which are used for which suppress coughing, and expectorants, which alleviate coughing, while enhancing the production of mucus and phlegm. Anti-coughing agents may ease the administration of inhaled aerosols. According to some embodiments, the at least one anti-coughing agent is selected from expectorants, antitussives or both. According to some embodiments, the at least one anti-coughing agent is selected from the group consisting of menthol, dextromethorphan, dextromethorphan hydrobromide, hydrocodone, caramiphen dextrorphan, 3-methoxymorphinan or morphinan-3-ol, carbetapentane, codeine, acetylcysteine and combinations thereof.

As exemplified herein (e.g. Example 4) the composition of the invention provide an effective dose of THC, which is comparable to the amount of THC delivered through the lungs, by smoking cannabis directly. Without wishing to be bound by any theory or mechanism of action, the high dosage of THC that reaches the lungs by inhaling the cannabinoid composition using an electronic cigarette is attributed to the small aerosol droplets, having MMAD within the range of about 0.2 to 4 microns. It is noted that such small droplets were maintained even at aerosol produced with high THC concentrations, of about 5%. Thus, high THC concentrations can be inhaled and reach the lungs using an electronic cigarette adapted for the aerosolization of aqueous solutions and the cannabinoid compositions disclosed herein.

According to some embodiments, the at least one cannabinoic acid or salt thereof is extracted from a plant material, wherein the plant is of cannabis genus. According to some embodiments, the plant material is selected from Cannabis indica, Cannabis sativa and cannabis species engineered to have high THC/THCA content. According to some embodiments, the cannabis species is a THCA enriched cannabis species.

According to some embodiments, the at least one cannabinoic acid or salt thereof comprises tetrahydrocannabinolic acid (THCA), cannabidiolic acid (CBDA), salts thereof or a combination thereof. According to some embodiments, the at least one cannabinoic acid or salt thereof comprises THCA or a salt thereof. According to some embodiments, the at least one cannabinoic acid or salt thereof comprises THCA-salt. According to some embodiments, the at least one cannabinoid compound comprises THCA-sodium salt.

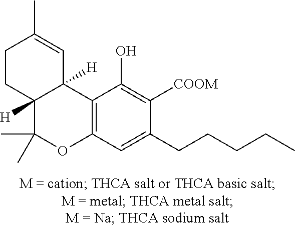

According to some embodiments, the cannabinoid composition is substantially devoid of organic solvents. As used herein, “substantially devoid” means that a preparation or composition according to the invention that generally contains less than 3% of the stated substance, such as less than 1% or less than 0.5%. According to some embodiments, the cannabinoid composition less than 10% w/w organic solvents. According to some embodiments, the cannabinoid composition less than 5% w/w organic solvents. According to some embodiments, the cannabinoid composition less than 1% w/w organic solvents. According to some embodiments, the cannabinoid composition less than 0.5% w/w organic solvents.

According to some embodiments, the cannabinoid composition is in liquid form. According to some embodiments, the cannabinoid composition comprises at least 50% w/w water. According to some embodiments, the cannabinoid composition comprises at least 75% w/w water. According to some embodiments, the cannabinoid composition comprises at least 90% w/w water. It is to be understood that the phrase “cannabinoid composition comprises at least 90% w/w water” means that each gram of the total composition includes at least 900 milligrams of water and at most 100 milligrams of materials other than water. According to some embodiments, the cannabinoid composition comprises more than 90% w/w water.

According to some embodiments, there is provided a process for preparing a cannabinoid composition. According to some embodiments, there is provided a process for preparing the cannabinoid composition disclosed herein.

According to some embodiments, the aqueous solution is prepared by a process comprising the steps of: (a) contacting cannabis plant material with an aqueous base, to form an aqueous solution comprising the at least one cannabinoic acid or salt thereof, and a water insoluble plant material; and (b) separating the aqueous solution comprising the at least one cannabinoic acid or salt thereof from the insoluble plant material. According to some embodiments, the aqueous solution is prepared by a process comprising the steps of: (a) contacting cannabis plant material with an aqueous base, to form a suspension comprising an aqueous solution of the at least one cannabinoic acid or salt thereof, and a water insoluble plant material; and (b) separating the aqueous solution comprising the at least one cannabinoic acid or salt thereof from the insoluble plant material.

The term “aqueous base” refers to any solution, emulsion or suspension comprising at least 50% water and having a pH above 8. According to some embodiments, the aqueous base comprises sodium hydroxide, potassium hydroxide, lithium hydroxide, magnesium hydroxide, sodium carbonate, potassium carbonate or a combination thereof. Each possibility represents a separate embodiment. According to some embodiments, the aqueous base is aqueous sodium hydroxide. According to some embodiments, the aqueous base is aqueous sodium hydroxide at a concentration in the range of 0.005M to 1M. According to some embodiments, the aqueous base comprises a hydroxide anion at a concentration in the range of 0.001M to 0.5M. According to some embodiments, the aqueous base comprises a hydroxide anion at a concentration in the range of 0.05M to 0.5M. According to some embodiments, the aqueous base comprises a hydroxide anion at a concentration in the range of 0.1M to 0.25M

According to some embodiments, the process further comprises a step of grinding the cannabis plant material prior to step (a).

According to some embodiments, the contacting of step (a) is maintained for at least 1 hour. According to some embodiments, the contacting of step (a) is maintained for at least 12 hours. According to some embodiments, the contacting of step (a) is maintained for at least 24 hours.

According to some embodiments, the separation of step (b) is performed by centrifugation.

According to some embodiments, step (a) further comprises applying pressure on the cannabis plant material in the aqueous base. According to some embodiments, step (a) further comprises macerating the cannabis plant material in the aqueous base.

According to some embodiments, the cannabis plant material of step (a) comprises tetrahydrocannabinolic acid (THCA). According to some embodiments, the cannabis plant material of step (a) comprises a THCA-enriched cannabis species.

According to some embodiments, the process is devoid of steps of extraction with an organic solvent.

According to some embodiments, the process further comprises the steps of: (c) adding an acid to the aqueous solution comprising the at least one cannabinoic acid or salt thereof at the form of a salt to a pH in the range of 1-5, thereby precipitating the at least one cannabinoic acid or salt thereof, at the form of an acid and forming an acidic aqueous solution; (d) separating the precipitated at least one cannabinoic acid from the acidic aqueous solution; and (e) dissolving the precipitated at least one cannabinoic acid in a second aqueous base at the form of a cannabinoic acid basic salt, thereby forming a purified aqueous solution comprising the at least one cannabinoic acid or salt thereof.

According to some embodiments, the acid is a mineral acid. According to some embodiments, the pH of the acidic aqueous solution of step (c) is in the range of 2 to 5.5. According to some embodiments, the pH of the acidic aqueous solution of step (c) is in the range of 2.5 to 5.5. According to some embodiments, the pH of the acidic aqueous solution of step (c) is in the range of 3 to 5. According to some embodiments, the pH of the acidic aqueous solution of step (c) is in the range of 3.5 to 4.5.

According to some embodiments, the second aqueous base of step (e) comprises a hydroxide anion at a concentration in the range of 0.01M to 0.5M.

According to some embodiments, there is provided a method of delivering a cannabinoid to a user of an electronic cigarette via inhalation, the method comprising the steps of: (i) providing the cannabinoid composition as disclosed herein; and (ii) aerosolizing the cannabinoid composition of step (a) with an electronic cigarette, to form an inhalable aerosol. According to some embodiments, the electronic cigarette is the electronic cigarette disclosed herein. According to some embodiments, there is provided a method of treating a medical condition amenable to treatment with THC, the method comprising the steps of: (i) providing a cannabinoid composition as disclosed herein; (ii) aerosolizing the cannabinoid composition of step (a) with an aerosol generating device; and (iii) delivering the inhalable aerosol of step (ii) to a subject in need thereof, thereby treating the medical condition amenable to treatment with THC. According to some embodiments, the aerosol generating device is the electronic cigarette disclosed herein.

According to some embodiments, the medical condition is selected from the group consisting of pain, impaired neurological function, inflammation, nausea, vomiting, convulsions, low appetite and glaucoma.

According to some embodiments, the inhalable aerosol is inhaled by the user of the electronic cigarette. According to some embodiments, the inhalable aerosol is inhaled by the subject. According to some embodiments, the method further comprises the step of inhaling the inhalable aerosol by a user. According to some embodiments, the method further comprises the step of inhaling the inhalable aerosol by the subject. According to some embodiments, the method further comprises the step of inhaling the inhalable aerosol by a user of an electronic cigarette. According to some embodiments, the delivering of the cannabinoid to a user comprises delivering of the cannabinoid to a user comprises delivering the cannabinoid to the respiratory system of the user. According to some embodiments, the delivering of the cannabinoid to the subject comprises delivering of the cannabinoid to a user comprises delivering the cannabinoid to the respiratory system of the subject.

It is to be understood that the embodiment related to cannabinoid composition and/or the pharmaceutical composition above, may apply for any of the methods disclosed herein. Specifically, wherein the method is for treatment of a subject, the cannabinoid composition may be a pharmaceutical composition, according to some embodiments.

As elaborated above, the pH of the cannabinoid composition is highly basic, whereas the pH of the aerosol produced therefrom is typically substantially neutral, according to some embodiments. This may be the result of the formation of the neutral compound THC from THCA basic salt. As detailed in Examples 1 and 2, the aerosol produced by the aerosolization (or a number of aerosolization events) of the cannabinoid composition may be collected, and its pH measured conveniently.

According to some embodiments, the aerosolizing of step (ii) comprises heating the cannabinoid composition of step (i) with the electronic cigarette. According to some embodiments, the aerosolizing of step (ii) comprises heating the cannabinoid composition of step (i) with the aerosol generating device.

According to some embodiments, step (ii) of the method of delivering a cannabinoid to a user of an electronic cigarette via inhalation, comprises: providing the electronic cigarette of the current disclosure, and aerosolizing the cannabinoid composition of step (a) with the electronic cigarette, to form the inhalable aerosol. According to some embodiments, step (ii) of the method of treating a medical condition amenable to treatment with THC, comprises: providing the electronic cigarette of the current disclosure, and aerosolizing the cannabinoid composition of step (a) with the aerosol generating device, to form the inhalable aerosol.

As detailed above, the methods of the current invention are effective in delivering THC to the respiratory system of the electronic cigarette user and/or to the respiratory system of the subject in need of treatment with said cannabinoid, according to some embodiments.

As used herein, “respiratory system” refers to the system of organs in the body responsible for the intake of oxygen and the expiration of carbon dioxide. The system generally includes all the air passages from the nose to the pulmonary alveoli. In mammals it is generally considered to include the lungs, bronchi, bronchioles, trachea, nasal passages, and diaphragm. For purposes of the present disclosure, delivery of a drug to the “respiratory system” indicates that a drug is delivered to one or more of the air passages of the respiratory system, in particular to the lungs.

The correlation between droplet size and deposition thereof in the respiratory tract has been established. Droplets around 10 micron in diameter are suitable for deposition in the oropharynx and the nasal area; droplets below around 4 micron in diameter are suitable for deposition in the central airways and may be especially beneficial for delivery of cannabinoid the subjects in a need thereof. The droplets formed by aerosolizing the cannabinoid composition of the current invention are small, having droplet size in the range of 0.1 to 5 micron, according to some embodiments.

According to some embodiments, there is provided an electronic cigarette cartridge comprising a liquid container, wherein the liquid container contains a cannabinoid compositions as disclosed herein. According to some embodiments, there is provided an electronic cigarette cartridge comprising a liquid container, wherein the liquid container contains a cannabinoid composition comprising an aqueous solution comprising at least one cannabinoic acid or salt thereof, wherein the aqueous solution has a pH of at least 9. According to some embodiments, there is provided an electronic cigarette comprising a liquid container, wherein the liquid container contains a cannabinoid composition as disclosed herein. According to some embodiments, there is provided an electronic cigarette comprising a liquid container, wherein the liquid container contains a cannabinoid composition comprising an aqueous solution comprising at least one cannabinoic acid or salt thereof, wherein the aqueous solution has a pH of at least 9. According to some embodiments, the electronic cigarette cartridge is cartridge 106 of electronic cigarette 100.

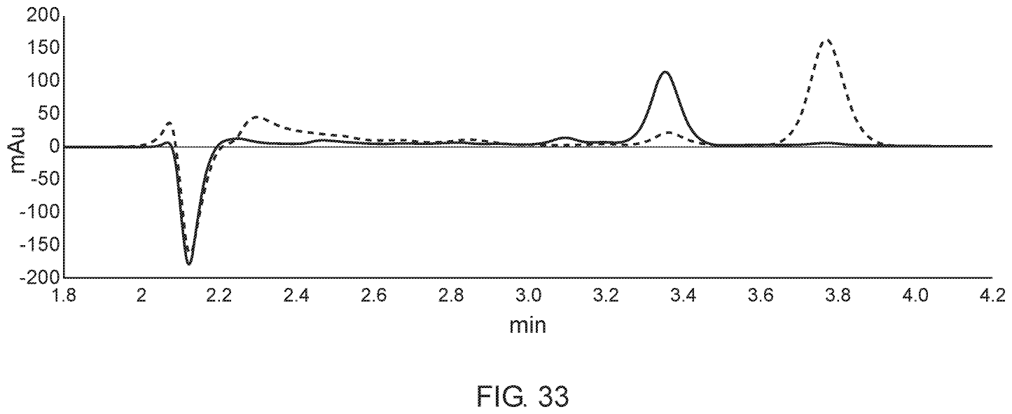

Specifically, as shown in Example 3, the cannabinoid composition of the current invention comprises THCA as a main component, and upon aerosolization, it undergoes decarboxylation, to form an aerosol comprising mainly THC. However, according to some embodiments, traces of THCA may still be present in the aerosol, as shown in FIG. 33. According to some embodiments, the aerosol composition is having a pH in the range of 5.5 to 7.5. Specifically, upon collection of the aerosol, the pH was measured to be substantially neutral, indicating the substantial disappearance of THC and formation of the pH neutral THC.

According to some embodiments, the aerosol composition comprises droplets having a mass median aerodynamic diameter (MMAD) of at most 50 microns. According to some embodiments, the aerosol composition comprises droplets having a mass median aerodynamic diameter (MMAD) of at most 40 microns. According to some embodiments, the aerosol composition comprises droplets having a mass median aerodynamic diameter (MMAD) of at most 30 microns. According to some embodiments, the aerosol composition comprises droplets having a mass median aerodynamic diameter (MMAD) of at most 20 microns. According to some embodiments, the aerosol composition comprises droplets having a mass median aerodynamic diameter (MMAD) of at most 10 microns. According to some embodiments, the aerosol composition comprises droplets having a mass median aerodynamic diameter (MMAD) of at most 8 microns. According to some embodiments, the aerosol composition comprises droplets having a mass median aerodynamic diameter (MMAD) of at most 6 microns. According to some embodiments, the aerosol composition comprises droplets having a mass median aerodynamic diameter (MMAD) of at most 5 microns.

It was surprisingly found that aerosolization of a formulation as disclosed herein, results in droplets having a mass median aerodynamic diameter (MMAD) sufficiently small so as to reach the lungs, rather than precipitate on their way thereto. The small droplets reaching the lungs enable efficient respiratory delivery of the cannabinoid(s). This is an overall advantage as maximizing the delivery of cannabinoid(s) to the lungs, while minimizing its deposition in the mouth and throat are considered highly beneficial.

The terms ‘droplet size’ and ‘mass median aerodynamic diameter’, also known as MMAD, as used herein are interchangeable. MMAD is commonly considered as the median particle diameter by mass. MMAD may be evaluated by plotting droplet size vs. the cumulative mass fraction (%) in the aerosol. MMAD may then be determined according to the interpolated droplet size corresponding to the point, where the cumulative mass fraction is 50%. This points represent the estimated values of particle sizes, above which the droplets are responsible to half to masses and below which the droplets are responsible to the other halves, in each solution.

According to some embodiments, the aerosol composition is prepared by aerosolizing the cannabinoid composition as disclosed herein. According to some embodiments, the aerosol composition is prepared by aerosolizing a cannabinoid composition using electronic cigarette 100.

### EXAMPLES

### Example 1: Preparation of Formulation for Inhalation

The formulation for inhalation analyzed in the experiments below included a clear basic aqueous solution of tetrahydrocannabinolic acid (THCA) adjusted to pH ˜11. The solution was prepared by grinding a 1000 gr sample of THCA-enriched cannabis species and placing the ground plant material in a glass vessel. About 12 L aqueous solution of 0.1M sodium hydroxide was added to the glass vessel and the mixture was left over night. All the material was then transferred from the glass vessel to a stainless-steel mesh and the plant material was macerated by application of physical pressure. The liquid contents were then centrifuged and the supernatant was collected as clear solution. The solution was visibly clear and its pH was measured to be about 11. The solution was measured to contain about 5% w/w sodium tetrahydrocannabinolate (tetrahydrocannabinolic acid sodium salt). The solution was ready for inhalation using an electronic cigarette.

The formulation was aerosolized from the electronic cigarette of the current invention. The aerosol was collected and its pH was measured to be substantially neutral, indicating that the THCA underwent decarboxylation to form the pH neutral compound THC in the aerosol.

### Example 2: Preparation of Formulation for Inhalation

The formulation for inhalation analyzed in the experiments below included a clear basic aqueous solution of tetrahydrocannabinolic acid (THCA) adjusted to pH ˜11. The solution was prepared by grinding a 1000 gr sample of THCA-enriched cannabis species and placing the ground plant material in a glass vessel. About 12 L aqueous solution of 0.1M sodium hydroxide was added to the glass vessel and the mixture was left over night. All the material was then transferred from the glass vessel to a stainless-steel mesh and the plant material was macerated by application of physical pressure. The liquid contents were then centrifuged and the supernatant was collected as clear solution. Thereafter, the clear solution was added concentrated hydrochloric acid until the pH reached about 4. As a result, tetrahydrocannabinolic acid started to precipitate. The formed suspension was centrifuged and the solids were separated. The centrifugation and solid separation steps were repeated twice more with sequential additions of water. The solid tetrahydrocannabinolic acid was solubilized in 0.01M aqueous sodium hydroxide. The solution was visibly clear and its pH was measured to be about 11. The solution was measured to contain about 5% w/w sodium tetrahydrocannabinolate (tetrahydrocannabinolic acid sodium salt). The solution was ready for inhalation with an electronic cigarette.

The formulation was aerosolized from the electronic cigarette of the current invention. The aerosol was collected and its pH was measured to be substantially neutral, indicating that the THCA underwent decarboxylation to form the pH neutral compound THC in the aerosol.

### Example 3: Analysis of the Formulation for Inhalation

The exemplary formulation solution was checked for the relative amounts of the cannabinoids THC and THCA in a Dionex ultimate 3000 HPLC system with the mobile phase being 90% acetonitrile/10% water/0.1% formic acid and the stationary phase being reverse phase C18 column. The column oven temperature was set to 35° C. and the flow was set to 1 ml/min. The UV detection was at 220 nm. The elution time were compared with elution times of THC and THCA as known in the literature.

FIG. 33 is showing two overlaying chromatograms. The dotted trend line represents the chromatogram resulting from the elution of the formulation of Example 1, without further processing, with the mobile phase being 90% acetonitrile/10% water/0.1% formic acid. This chromatogram shows a large peak at retention time of about 3.8 minutes, which is comparable with the literature value of THCA at similar elution conditions, and a small peak at about 3.35 minutes, which is comparable with the literature value of THC at similar elution conditions. Therefore, it is concluded that the formulation of the current invention comprises mainly THCA, which in basic conditions appears as a basic salt.

The second chromatogram of FIG. 33, represented by a full line, resulted from the elution in the conditions above of an aerosol collected from the electronic cigarette of the current invention, after aerosolizing the formulation of Example 1. This chromatogram of FIG. 33 shows a large peak at retention time of about 3.35, which is indicative of THC; and a very small peak at retention time of about 3.8, indicating THCA. Therefore, it is concluded that the aerosol formed by heating the formulation of the current invention with the current electronic cigarette comprises mainly THC, which is the active cannabinoid form. This also may explain the neutral pH of the aerosol. Without wishing to be bound by any theory or mechanism of action, upon the heating of the THCA, the majority thereof is decarboxylated to for THC. Since the heating process is rapid, some of the THCA is evaporated before decarboxylating and thus it appears in the aerosol.

### Example 4: Mass Distribution on Impactor Parts

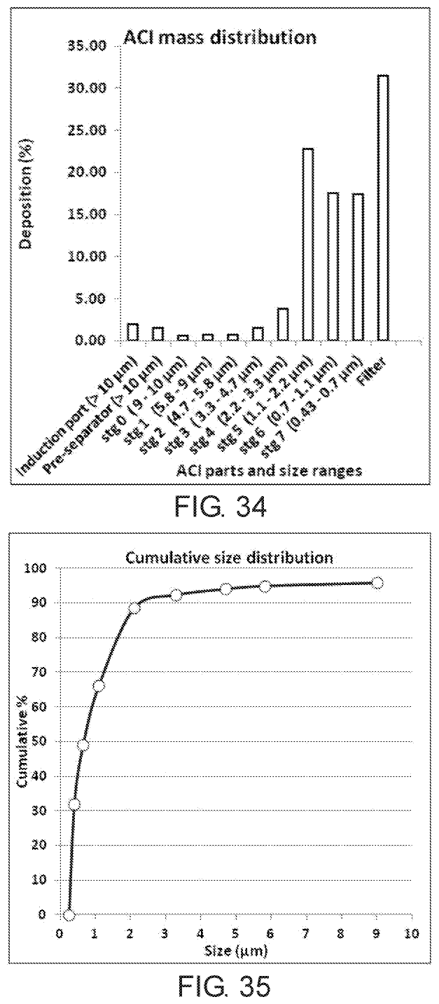

Particle size distribution testing was conducted using cascade impactor validated method with the basic aqueous solution of tetrahydrocannabinolic acid of Example 1. The limits for the median diameter range from 0.4 to 0.8 micron and the limit on the sub 5 micron particles/droplets was set at 90%. The results are presented in FIG. 34 and relate to the formulation of example 1 aerosolized with the electronic cigarette of the current invention.

Relative mass of the aerosolized solution was measured against its particle size, which was measured between 0.43 micrometers and over 10 micrometers.

FIG. 34 is a chart representing Mass Distribution on Impactor parts in an aerosol depicting the relative mass of the aerosol in each particle diameter size group, where the particle diameter groups are 0.43 to 0.7 microns; 0.7 to 1.1 microns; 1.1 to 2.2 microns; 2.2 to 3.3 microns; 3.3 to 4.7 microns; 4.7 to 5.8 microns; 5.8 to 9 microns; and over 10 microns.

As can be seen in FIG. 34, the majority of aerosol mass was provided in droplets having diameters in the range of 0.43 to 2.2 microns.

Finally, FIG. 35 is a chart representing cumulative Mass Distribution of the aerosol in the experiment. It depicts the cumulative mass fraction vs. the droplet size in micrometers. The 50% mark in the cumulative percentage axis represents the estimated value of particle size, above which the droplets are responsible to half to mass and below which the droplets are responsible to the other half. Again, it is seen that half of the mass was delivered in droplets having diameters below about 0.8 microns.

It is understood that aspect and embodiments described herein include “consisting” and/or “consisting essentially of” aspects and embodiments. As used herein, the singular form “a”, “an”, and “the” includes plural references unless indicated otherwise.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1.-50. (canceled)

51. An electronic cigarette comprising:
a cartridge having a first end and a second end, the cartridge comprising:
an evaporation heater configured to generate heat and to evaporate a liquid from a surface thereof;
a liquid drawing element;
a liquid container;
an outlet; and
an actuator having a first end and a second end, the actuator comprising a processing unit,
wherein the first end of the actuator is connectable with the second end of the cartridge, wherein the electronic cigarette further comprises a first trigger configured to generate a first trigger activation signal, and a liquid deposition mechanism comprising the liquid drawing element and the liquid container, wherein the liquid drawing element is spaced apart from the evaporation heater in at least a first state of the electronic cigarette, and wherein the liquid deposition mechanism is configured to transfer a discrete volume of an aqueous formulation from the liquid drawing element to the evaporation heater in a second state of the electronic cigarette, wherein the liquid drawing element is in contact with the liquid container in both the first state of the electronic cigarette and the second state of the electronic cigarette, wherein the processing unit is configured to receive at least one operation signal and to control operations of at least one of the evaporation heater and the liquid deposition mechanism upon receiving the at least one operation signal, wherein the at least one operation signal comprises the first trigger activation signal.

52. The electronic cigarette of claim 51, wherein the processing unit is configured to control the operation of the liquid deposition mechanism to prevent transfer of liquids from the liquid drawing element to the evaporation heater in the first state of the electronic cigarette, wherein the electronic cigarette is configured to intermittently switch between the first state and the second state thereof, through the processing unit sequentially controlling the operation of the liquid deposition mechanism to (a) prevent transfer of liquids from the liquid drawing element to the evaporation heater in the first state of the electronic cigarette; and (b) transfer a discrete volume of an aqueous formulation from the liquid drawing element to the evaporation heater in the second state of the electronic cigarette.

53. The electronic cigarette of claim 51, wherein the evaporation heater is flat and comprises a first flat surface facing the outlet and a second flat surface facing the fluid deposition mechanism, and wherein the liquid deposition mechanism is configured to transfer a discrete volume of an aqueous formulation from the liquid drawing element to the second flat surface of the evaporation heater in the second state of the electronic cigarette.

54. The electronic cigarette of claim 53, wherein the evaporation heater is at least partially permeable to the aqueous formulation, and configured to receive the discrete volume of aqueous formulation from the liquid drawing element to the second flat surface thereof, and to evaporate the aqueous formulation through the first flat surface thereof in the second state of the electronic cigarette, such that the evaporated aqueous formulation is released through the outlet.

55. The electronic cigarette of claim 51, wherein the evaporation heater comprises an elongated heat conductive coil having a first end, a second end and a main body portion extending there between in a spiraloid path to form a two-dimensional shape having a first flat surface facing the outlet and a second flat surface facing the liquid drawing element, wherein the spiraloid path forms inner tracks between portions of the main body of the elongated heat conductive coil.

56. The electronic cigarette of claim 51, wherein the discrete volume of the aqueous formulation has a volume in the range of 2 μL to 40 μL.

57. The electronic cigarette of claim 51, wherein the first trigger comprises a user interface, which provides options to a user for determining at least one control parameter, by which the processing unit controls the liquid deposition mechanism, wherein the at least one control parameter is selected from fluid deposition frequency and fluid deposition duty cycle.

58. The electronic cigarette of claim 57, wherein the processing unit is configured to control the liquid deposition mechanism in a fluid deposition frequency in the range of 1 Hz to 100 Hz, wherein the at least one control parameter comprises at least two fluid deposition frequencies in the range of 1 Hz to 100 Hz.

59. The electronic cigarette of claim 57, wherein the processing unit is configured to control the liquid deposition mechanism in a duty cycle in the range of 10% to 50%, wherein the at least one control parameter comprises at least two duty cycles in the range of 10% to 50%.

60. The electronic cigarette of claim 51, wherein the liquid deposition mechanism further comprises a biasing element, configured to trigger a dislocation of at least a portion of the liquid drawing element between a first position in the first state of the electronic cigarette and a second position in the second state of the electronic cigarette, wherein the liquid drawing element is spaced apart from the evaporation heater in the first position, and wherein the liquid drawing element is in contact with the evaporation heater in the second position.

61. The electronic cigarette of claim 60, wherein the biasing element is positioned between the liquid drawing element and the second end of the actuator, and is configured to dislocate the portion of the liquid drawing element from the first position in the first state of the electronic cigarette in the direction of the first end of the cartridge, towards the second position in the second state of the electronic cigarette, and further configured to trigger dislocation of the liquid drawing element from the second position in the second state of the electronic cigarette in the direction of the second end of the actuator, towards the first position in the first state of the electronic cigarette.

62. The electronic cigarette claim 60, wherein the liquid drawing element is flexible and comprises at least first portion and a second portion, wherein the second portion of the liquid drawing element is in contact with the liquid container in both the first state of the electronic cigarette and the second state of the electronic cigarette, and wherein the biasing element is configured to trigger a dislocation of the first portion of the liquid drawing element between the first position in the first state of the electronic cigarette and the second position in the second state of the electronic cigarette, wherein the first portion of the liquid drawing element is spaced apart from the evaporation heater in the first position, and wherein the first portion of liquid drawing element is in contact with the evaporation heater in the second position.

63. The electronic cigarette of claim 60, wherein the liquid drawing element comprises fabric, cloth, wool, felt, sponge, foam, cellulose, yarn, microfiber or a combination thereof.

64. The electronic cigarette of claim 60, wherein the biasing element comprises a solenoid actuator, a rod and a solenoid plunger head, wherein the rod has a first end and a second end, wherein the second end is connected to the solenoid actuator, and the first end is connected to the solenoid plunger head, wherein the solenoid actuator is configured to dislocate the solenoid plunger head between a first position and a second position, wherein in the second state of the electronic cigarette, the solenoid plunger head is in the second position thereof and is pressing the portion of the liquid drawing element against the evaporation heater, and in the first state of the electronic cigarette, the solenoid plunger head is in the first position thereof and the liquid drawing element is spaced apart from the evaporation heater.

65. The electronic cigarette of claim 64, wherein the actuator further comprises a liquid deposition mechanism housing, wherein the liquid deposition mechanism housing accommodates the solenoid actuator, wherein the rod extends from the solenoid actuator in the direction of the first end of the cartridge, wherein when the cartridge and the actuator are assembled, the solenoid plunger head resides inside the cartridge, between the solenoid actuator and the evaporation heater.

66. The electronic cigarette of claim 60, wherein the solenoid actuator is configured to receive electric current and to generate axial movement of the solenoid plunger head upon receiving the electric current, wherein the axial movement is along an axis perpendicular to the evaporation heater, between the first position of the solenoid plunger head in the first state of the electronic cigarette and the second position of the solenoid plunger head in the second state of the electronic cigarette.

67. The electronic cigarette of claim 51, wherein the liquid deposition mechanism further comprises a spraying mechanism, located within the cartridge and configured to create a spray from the aqueous formulation, wherein the spraying mechanism is in contact with the liquid drawing element and spaced apart from the evaporation heater in both the first state of the electronic cigarette and the second state of the electronic cigarette.

68. The electronic cigarette of claim 67, wherein the spraying mechanism is located between the liquid drawing element and the evaporation heater, wherein in the first state of the electronic cigarette the spraying mechanism does not create a spray, and wherein in the second state of the electronic cigarette, the spray is sprayed from the spraying mechanism in the direction of the first end of the actuator and contacts the evaporation heater.

69. The electronic cigarette of claim 67, wherein the liquid deposition mechanism further comprises a liquid deposition mechanism housing, and the spraying mechanism comprises a piezo disc configured to create the spray from the aqueous formulation, wherein the piezo disc is in contact with the liquid drawing element and spaced apart from the evaporation heater in both the first state of the electronic cigarette and the second state of the electronic cigarette, wherein the piezo disc is accommodated within the liquid deposition mechanism housing.

70. The electronic cigarette of claim 69, wherein the piezo disc is configured to convert electric current to vibrations having resonant frequency, which creates the spray from the aqueous formulation, wherein the resonant frequency is in the range of 100 KHz-10 MHz.
